SEQUENCE LISTING (557 sequences)

SEQ ID NO: 1 - RNA, 20 nt, Artificial Sequence
  source: /note=“Description of Artificial Sequence: Synthetic oligonucleotide”
aguccugguauccucuauga

SEQ ID NO: 2 - RNA, 20 nt, Artificial Sequence
  source: /note=“Description of Artificial Sequence: Synthetic oligonucleotide”
aauuagcaguauccucuugg

SEQ ID NO: 3 - RNA, 20 nt, Artificial Sequence
  source: /note=“Description of Artificial Sequence: Synthetic oligonucleotide”
agaauaaauuagagaaaaac

SEQ ID NO: 4 - RNA, 20 nt, Artificial Sequence
  source: /note=“Description of Artificial Sequence: Synthetic oligonucleotide”
aaaaauuagcaguauccucu

SEQ ID NO: 5 - RNA, 20 nt, Artificial Sequence
  source: /note=“Description of Artificial Sequence: Synthetic oligonucleotide”
aaaauuagcaguauccucuu

SEQ ID NO: 6 - RNA, 20 nt, Artificial Sequence
  source: /note=“Description of Artificial Sequence: Synthetic oligonucleotide”
aaaaacuggaaugacugaau

SEQ ID NO: 7 - RNA, 20 nt, Artificial Sequence
  source: /note=“Description of Artificial Sequence: Synthetic oligonucleotide”
cucccaucauagaggauacc

SEQ ID NO: 8 - RNA, 20 nt, Artificial Sequence
  source: /note=“Description of Artificial Sequence: Synthetic oligonucleotide”
ggagaaggaaacuagcuaaa

SEQ ID NO: 9 - RNA, 20 nt, Artificial Sequence
  source: /note=“Description of Artificial Sequence: Synthetic oligonucleotide”
guuuccuucucccaucauag

SEQ ID NO: 10 - RNA, 20 nt, Artificial Sequence
  source: /note=“Description of Artificial Sequence: Synthetic oligonucleotide”
gggagaaggaaacuagcuaa

SEQ ID NO: 11 - RNA, 20 nt, Artificial Sequence
  source: /note=“Description of Artificial Sequence: Synthetic oligonucleotide”
cacuggagcuagagacaaga

SEQ ID NO: 12 - RNA, 20 nt, Artificial Sequence
  source: /note=“Description of Artificial Sequence: Synthetic oligonucleotide”
agagacaagaagguaaaaaa

SEQ ID NO: 13 - RNA, 20 nt, Artificial Sequence
  source: /note=“Description of Artificial Sequence: Synthetic oligonucleotide”
aaauuagcaguauccucuug

SEQ ID NO: 14 - RNA, 20 nt, Artificial Sequence
  source: /note=“Description of Artificial Sequence: Synthetic oligonucleotide”
guccugguauccucuaugau

SEQ ID NO: 15 - RNA, 20 nt, Artificial Sequence
  source: /note=“Description of Artificial Sequence: Synthetic oligonucleotide”
guauccucuaugaugggaga

SEQ ID NO: 16 - RNA, 20 nt, Artificial Sequence
  source: /note=“Description of Artificial Sequence: Synthetic oligonucleotide”
ccuagccagccgccggcccc

SEQ ID NO: 17 - RNA, 20 nt, Artificial Sequence
  source: /note=“Description of Artificial Sequence: Synthetic oligonucleotide”
auuaagcagcaguauccucu

SEQ ID NO: 18 - RNA, 20 nt, Artificial Sequence
  source: /note=“Description of Artificial Sequence: Synthetic oligonucleotide”
uauccagugaggccaggggc

SEQ ID NO: 19 - RNA, 20 nt, Artificial Sequence
  source: /note=“Description of Artificial Sequence: Synthetic oligonucleotide”
cauugagauaguguggggaa

SEQ ID NO: 20 - RNA, 20 nt, Artificial Sequence
  source: /note=“Description of Artificial Sequence: Synthetic oligonucleotide”
ccagugaggccaggggccgg

SEQ ID NO: 21 - RNA, 20 nt, Artificial Sequence
  source: /note=“Description of Artificial Sequence: Synthetic oligonucleotide”
guggggaaggggcccccaag

SEQ ID NO: 22 - RNA, 20 nt, Artificial Sequence
  source: /note=“Description of Artificial Sequence: Synthetic oligonucleotide”
agaauaaauuagagaaaaau

SEQ ID NO: 23 - RNA, 20 nt, Artificial Sequence
  source: /note=“Description of Artificial Sequence: Synthetic oligonucleotide”
ccaggggccggcggcuggcu

SEQ ID NO: 24 - RNA, 20 nt, Artificial Sequence
  source: /note=“Description of Artificial Sequence: Synthetic oligonucleotide”
ugaggccaggggccggcggc

SEQ ID NO: 25 - RNA, 20 nt, Artificial Sequence
  source: /note=“Description of Artificial Sequence: Synthetic oligonucleotide”
caguuccacacacucgcuuc

SEQ ID NO: 26 - RNA, 20 nt, Artificial Sequence
  source: /note=“Description of Artificial Sequence: Synthetic oligonucleotide”
ccgccggccccuggccucac

SEQ ID NO: 27 - RNA, 20 nt, Artificial Sequence
  source: /note=“Description of Artificial Sequence: Synthetic oligonucleotide”
guuugccuugucaaggcuau

SEQ ID NO: 28 - RNA, 20 nt, Artificial Sequence
  source: /note=“Description of Artificial Sequence: Synthetic oligonucleotide”
ggcuagggaugaagaauaaa

SEQ ID NO: 29 - RNA, 20 nt, Artificial Sequence
  source: /note=“Description of Artificial Sequence: Synthetic oligonucleotide”
agaaguccugguaucuucua

SEQ ID NO: 30 - RNA, 20 nt, Artificial Sequence
  source: /note=“Description of Artificial Sequence: Synthetic oligonucleotide”
caggggccggcggcuggcua

SEQ ID NO: 31 - RNA, 20 nt, Artificial Sequence
  source: /note=“Description of Artificial Sequence: Synthetic oligonucleotide”
acuggauacucuaagacuau

SEQ ID NO: 32 - RNA, 20 nt, Artificial Sequence
  source: /note=“Description of Artificial Sequence: Synthetic oligonucleotide”
uuaagcagcaguauccucuu

SEQ ID NO: 33 - RNA, 20 nt, Artificial Sequence
  source: /note=“Description of Artificial Sequence: Synthetic oligonucleotide”
cccuggcuaaacuccaccca

SEQ ID NO: 34 - RNA, 20 nt, Artificial Sequence
  source: /note=“Description of Artificial Sequence: Synthetic oligonucleotide”
aaaaauuggaaugacugaau

SEQ ID NO: 35 - RNA, 20 nt, Artificial Sequence
  source: /note=“Description of Artificial Sequence: Synthetic oligonucleotide”
uuagaguauccagugaggcc

SEQ ID NO: 36 - RNA, 20 nt, Artificial Sequence
  source: /note=“Description of Artificial Sequence: Synthetic oligonucleotide”
cccauggguggaguuuagcc

SEQ ID NO: 37 - RNA, 20 nt, Artificial Sequence
  source: /note=“Description of Artificial Sequence: Synthetic oligonucleotide”
aggcaaggcuggccaaccca

SEQ ID NO: 38 - RNA, 20 nt, Artificial Sequence
  source: /note=“Description of Artificial Sequence: Synthetic oligonucleotide”
uagaguauccagugaggcca

SEQ ID NO: 39 - RNA, 20 nt, Artificial Sequence
  source: /note=“Description of Artificial Sequence: Synthetic oligonucleotide”
uaucugucugaaacgguccc

SEQ ID NO: 40 - RNA, 20 nt, Artificial Sequence
  source: /note=“Description of Artificial Sequence: Synthetic oligonucleotide”
auugagauaguguggggaag

SEQ ID NO: 41 - RNA, 20 nt, Artificial Sequence
  source: /note=“Description of Artificial Sequence: Synthetic oligonucleotide”
cuucaucccuagccagccgc

SEQ ID NO: 42 - RNA, 20 nt, Artificial Sequence
  source: /note=“Description of Artificial Sequence: Synthetic oligonucleotide”
gcuauuggucaaggcaaggc

SEQ ID NO: 43 - RNA, 20 nt, Artificial Sequence
  source: /note=“Description of Artificial Sequence: Synthetic oligonucleotide”
augcaaauaucugucugaaa

SEQ ID NO: 44 - RNA, 20 nt, Artificial Sequence
  source: /note=“Description of Artificial Sequence: Synthetic oligonucleotide”
gcauugagauagugugggga

SEQ ID NO: 45 - RNA, 20 nt, Artificial Sequence
  source: /note=“Description of Artificial Sequence: Synthetic oligonucleotide”
uggucaaguuugccuuguca

SEQ ID NO: 46 - RNA, 20 nt, Artificial Sequence
  source: /note=“Description of Artificial Sequence: Synthetic oligonucleotide”
gggagaagaaaacuagcuaa

SEQ ID NO: 47 - RNA, 20 nt, Artificial Sequence
  source: /note=“Description of Artificial Sequence: Synthetic oligonucleotide”
ggcaaggcuggccaacccau

SEQ ID NO: 48 - RNA, 20 nt, Artificial Sequence
  source: /note=“Description of Artificial Sequence: Synthetic oligonucleotide”
acggcugacaaaagaagucc

SEQ ID NO: 49 - RNA, 20 nt, Artificial Sequence
  source: /note=“Description of Artificial Sequence: Synthetic oligonucleotide”
cgaguguguggaacugcuga

SEQ ID NO: 50 - RNA, 20 nt, Artificial Sequence
  source: /note=“Description of Artificial Sequence: Synthetic oligonucleotide”
ccuggcuaaacuccacccau

SEQ ID NO: 51 - RNA, 20 nt, Artificial Sequence
  source: /note=“Description of Artificial Sequence: Synthetic oligonucleotide”
ggagaagaaaacuagcuaaa

SEQ ID NO: 52 - RNA, 20 nt, Artificial Sequence
  source: /note=“Description of Artificial Sequence: Synthetic oligonucleotide”
cucccaccauagaagauacc

SEQ ID NO: 53 - RNA, 20 nt, Artificial Sequence
  source: /note=“Description of Artificial Sequence: Synthetic oligonucleotide”
cuugucaaggcuauugguca

SEQ ID NO: 54 - RNA, 20 nt, Artificial Sequence
  source: /note=“Description of Artificial Sequence: Synthetic oligonucleotide”
aguccugguaucuucuaugg

SEQ ID NO: 55 - RNA, 20 nt, Artificial Sequence
  source: /note=“Description of Artificial Sequence: Synthetic oligonucleotide”
auauuugcauugagauagug

SEQ ID NO: 56 - RNA, 20 nt, Artificial Sequence
  source: /note=“Description of Artificial Sequence: Synthetic oligonucleotide”
gcuaaacuccacccaugggu

SEQ ID NO: 57 - RNA, 20 nt, Artificial Sequence
  source: /note=“Description of Artificial Sequence: Synthetic oligonucleotide”
acguuccagaagcgagugug

SEQ ID NO: 58 - RNA, 20 nt, Artificial Sequence
  source: /note=“Description of Artificial Sequence: Synthetic oligonucleotide”
guccugguaucuucuauggu

SEQ ID NO: 59 - RNA, 20 nt, Artificial Sequence
  source: /note=“Description of Artificial Sequence: Synthetic oligonucleotide”
uauuugcauugagauagugu

SEQ ID NO: 60 - RNA, 20 nt, Artificial Sequence
  source: /note=“Description of Artificial Sequence: Synthetic oligonucleotide”
uaagcagcaguauccucuug

SEQ ID NO: 61 - RNA, 20 nt, Artificial Sequence
  source: /note=“Description of Artificial Sequence: Synthetic oligonucleotide”
ggaaugacugaaucggaaca

SEQ ID NO: 62 - RNA, 20 nt, Artificial Sequence
  source: /note=“Description of Artificial Sequence: Synthetic oligonucleotide”
cuugaccaauagccuugaca

SEQ ID NO: 63 - RNA, 20 nt, Artificial Sequence
  source: /note=“Description of Artificial Sequence: Synthetic oligonucleotide”
caaggcuauuggucaaggca

SEQ ID NO: 64 - RNA, 20 nt, Artificial Sequence
  source: /note=“Description of Artificial Sequence: Synthetic oligonucleotide”
aaggcuggccaacccauggg

SEQ ID NO: 65 - RNA, 20 nt, Artificial Sequence
  source: /note=“Description of Artificial Sequence: Synthetic oligonucleotide”
acucgcuucuggaacgucug

SEQ ID NO: 66 - RNA, 20 nt, Artificial Sequence
  source: /note=“Description of Artificial Sequence: Synthetic oligonucleotide”
auuugcauugagauagugug

SEQ ID NO: 67 - RNA, 20 nt, Artificial Sequence
  source: /note=“Description of Artificial Sequence: Synthetic oligonucleotide”
acugaaucggaacaaggcaa

SEQ ID NO: 68 - RNA, 20 nt, Artificial Sequence
  source: /note=“Description of Artificial Sequence: Synthetic oligonucleotide”
ccauggguggaguuuagcca

SEQ ID NO: 69 - RNA, 20 nt, Artificial Sequence
  source: /note=“Description of Artificial Sequence: Synthetic oligonucleotide”
aagcagcaguauccucuugg

SEQ ID NO: 70 - RNA, 20 nt, Artificial Sequence
  source: /note=“Description of Artificial Sequence: Synthetic oligonucleotide”
agaguauccagugaggccag

SEQ ID NO: 71 - RNA, 20 nt, Artificial Sequence
  source: /note=“Description of Artificial Sequence: Synthetic oligonucleotide”
gaguguguggaacugcugaa

SEQ ID NO: 72 - RNA, 20 nt, Artificial Sequence
  source: /note=“Description of Artificial Sequence: Synthetic oligonucleotide”
uagucuuagaguauccagug

SEQ ID NO: 73 - protein, 835 aa, Homo sapiens
MSRRKQGKPQHLSKREFSPEPLEAILTDDEPDHGPLGAPEGDHDLLTCGQCQMNFPLGDILIFIEHKRKQCNGSLCLEKAVDKPPSPSPIEMKKASNPVEVGIQVTPEDDDCLSTSSRGICPKQEHIADKLLHWRGLSSPRSAHGALIPTPGMSAEYAPQGICKDEPSSYTCTTCKQPFTSAWFLLQHAQNTHGLRIYLESEHGSPLTPRVGIPSGLGAECPSQPPLHGIHIADNNPFNLLRIPGSVSREASGLAEGRFPPTPPLFSPPPRHHLDPHRIERLGAEEMALATHHPSAFDRVLRLNPMAMEPPAMDFSRRLRELAGNTSSPPLSPGRPSPMQRLLQPFQPGSKPPFLATPPLPPLQSAPPPSQPPVKSKSCEFCGKTFKFQSNLVVHRRSHTGEKPYKCNLCDHACTQASKLKRHMKTHMHKSSPMTVKSDDGLSTASSPEPGTSDLVGSASSALKSVVAKFKSENDPNLIPENGDEEEEEDDEEEEEEEEEEEEELTESERVDYGFGLSLEAARHHENSSRGAVVGVGDESRALPDVMQGMVLSSMQHFSEAFHQVLGEKHKRGHLAEAEGHRDTCDEDSVAGESDRIDDGTVNGRGCSPGESASGGLSKKLLLGSPSSLSPFSKRIKLEKEFDLPPAAMPNTENVYSQWLAGYAASRQLKDPFLSFGDSRQSPFASSSEHSSENGSLRFSTPPGELDGGISGRSGTGSGGSTPHISGPGPGRPSSKEGRRSDTCEYCGKVFKNCSNLTVHRRSHTGERPYKCELCNYACAQSSKLTRHMKTHGQVGKDVYKCEICKMPFSVYSTLEKHMKKWHSDRVLNNDIKTE

SEQ ID NO: 74-85 - skipped (12 entries)

SEQ ID NO: 86 - RNA, 20 nt, Artificial Sequence
  source: /note=“Description of Artificial Sequence: Synthetic oligonucleotide”
ucuuaaaccaaccugcucac

SEQ ID NO: 87 - RNA, 20 nt, Artificial Sequence
  source: /note=“Description of Artificial Sequence: Synthetic oligonucleotide”
cagguugguuuaagauaagc

SEQ ID NO: 88 - RNA, 20 nt, Artificial Sequence
  source: /note=“Description of Artificial Sequence: Synthetic oligonucleotide”
agguugguuuaagauaagca

SEQ ID NO: 89 - RNA, 20 nt, Artificial Sequence
  source: /note=“Description of Artificial Sequence: Synthetic oligonucleotide”
uuaagggaauaguggaauga

SEQ ID NO: 90 - RNA, 20 nt, Artificial Sequence
  source: /note=“Description of Artificial Sequence: Synthetic oligonucleotide”
agggcaaguuaagggaauag

SEQ ID NO: 91 - RNA, 20 nt, Artificial Sequence
  source: /note=“Description of Artificial Sequence: Synthetic oligonucleotide”
cccuuaacuugcccugagau

SEQ ID NO: 92 - RNA, 20 nt, Artificial Sequence
  source: /note=“Description of Artificial Sequence: Synthetic oligonucleotide”
ccaaucucagggcaaguuaa

SEQ ID NO: 93 - RNA, 20 nt, Artificial Sequence
  source: /note=“Description of Artificial Sequence: Synthetic oligonucleotide”
gccaaucucagggcaaguua

SEQ ID NO: 94 - RNA, 20 nt, Artificial Sequence
  source: /note=“Description of Artificial Sequence: Synthetic oligonucleotide”
ugacagaacagccaaucuca

SEQ ID NO: 95 - RNA, 20 nt, Artificial Sequence
  source: /note=“Description of Artificial Sequence: Synthetic oligonucleotide”
augacagaacagccaaucuc

SEQ ID NO: 96 - RNA, 20 nt, Artificial Sequence
  source: /note=“Description of Artificial Sequence: Synthetic oligonucleotide”
gagauauguagaggagaaca

SEQ ID NO: 97 - RNA, 20 nt, Artificial Sequence
  source: /note=“Description of Artificial Sequence: Synthetic oligonucleotide”
ggagauauguagaggagaac

SEQ ID NO: 98 - RNA, 20 nt, Artificial Sequence
  source: /note=“Description of Artificial Sequence: Synthetic oligonucleotide”
ugcgguggggagauauguag

SEQ ID NO: 99 - RNA, 20 nt, Artificial Sequence
  source: /note=“Description of Artificial Sequence: Synthetic oligonucleotide”
cugcugaaagagaugcggug

SEQ ID NO: 100 - RNA, 20 nt, Artificial Sequence
  source: /note=“Description of Artificial Sequence: Synthetic oligonucleotide”
acugcugaaagagaugcggu

SEQ ID NO: 101 - RNA, 20 nt, Artificial Sequence
  source: /note=“Description of Artificial Sequence: Synthetic oligonucleotide”
aacugcugaaagagaugcgg

SEQ ID NO: 102 - RNA, 20 nt, Artificial Sequence
  source: /note=“Description of Artificial Sequence: Synthetic oligonucleotide”
aacaacugcugaaagagaug

SEQ ID NO: 103 - RNA, 20 nt, Artificial Sequence
  source: /note=“Description of Artificial Sequence: Synthetic oligonucleotide”
ucugcaaaaaugaaacuagg

SEQ ID NO: 104 - RNA, 20 nt, Artificial Sequence
  source: /note=“Description of Artificial Sequence: Synthetic oligonucleotide”
acuucugcaaaaaugaaacu

SEQ ID NO: 105 - RNA, 20 nt, Artificial Sequence
  source: /note=“Description of Artificial Sequence: Synthetic oligonucleotide”
cauuuuugcagaaguguuuu

SEQ ID NO: 106 - RNA, 20 nt, Artificial Sequence
  source: /note=“Description of Artificial Sequence: Synthetic oligonucleotide”
aguguuuuaggcuaauauag

SEQ ID NO: 107 - RNA, 20 nt, Artificial Sequence
  source: /note=“Description of Artificial Sequence: Synthetic oligonucleotide”
uuggagacaaaaaucucuag

SEQ ID NO: 108 - RNA, 20 nt, Artificial Sequence
  source: /note=“Description of Artificial Sequence: Synthetic oligonucleotide”
ucuagagauuuuugucucca

SEQ ID NO: 109 - RNA, 20 nt, Artificial Sequence
  source: /note=“Description of Artificial Sequence: Synthetic oligonucleotide”
cuagagauuuuugucuccaa

SEQ ID NO: 110 - RNA, 20 nt, Artificial Sequence
  source: /note=“Description of Artificial Sequence: Synthetic oligonucleotide”
gucuccaagggaauuuugag

SEQ ID NO: 111 - RNA, 20 nt, Artificial Sequence
  source: /note=“Description of Artificial Sequence: Synthetic oligonucleotide”
ccaagggaauuuugagaggu

SEQ ID NO: 112 - RNA, 20 nt, Artificial Sequence
  source: /note=“Description of Artificial Sequence: Synthetic oligonucleotide”
ccaaccucucaaaauucccu

SEQ ID NO: 113 - RNA, 20 nt, Artificial Sequence
  source: /note=“Description of Artificial Sequence: Synthetic oligonucleotide”
ggaauuuugagagguuggaa

SEQ ID NO: 114 - RNA, 20 nt, Artificial Sequence
  source: /note=“Description of Artificial Sequence: Synthetic oligonucleotide”
ugcuugcuuccuccuucuuu

SEQ ID NO: 115 - RNA, 20 nt, Artificial Sequence
  source: /note=“Description of Artificial Sequence: Synthetic oligonucleotide”
aagaauuuaccaaaagaagg

SEQ ID NO: 116 - RNA, 20 nt, Artificial Sequence
  source: /note=“Description of Artificial Sequence: Synthetic oligonucleotide”
aggaagaauuuaccaaaaga

SEQ ID NO: 117 - RNA, 20 nt, Artificial Sequence
  source: /note=“Description of Artificial Sequence: Synthetic oligonucleotide”
aaaaauuagaguuuuauuau

SEQ ID NO: 118 - RNA, 20 nt, Artificial Sequence
  source: /note=“Description of Artificial Sequence: Synthetic oligonucleotide”
uuuuuuaaauauucuuuuaa

SEQ ID NO: 119 - RNA, 20 nt, Artificial Sequence
  source: /note=“Description of Artificial Sequence: Synthetic oligonucleotide”
uauuuaccaguuauugaaau

SEQ ID NO: 120 - RNA, 20 nt, Artificial Sequence
  source: /note=“Description of Artificial Sequence: Synthetic oligonucleotide”
ccaguuauugaaauagguuc

SEQ ID NO: 121 - RNA, 20 nt, Artificial Sequence
  source: /note=“Description of Artificial Sequence: Synthetic oligonucleotide”
ccagaaccuauuucaauaac

SEQ ID NO: 122 - RNA, 20 nt, Artificial Sequence
  source: /note=“Description of Artificial Sequence: Synthetic oligonucleotide”
uucuggaaacaugaauuuua

SEQ ID NO: 123 - RNA, 20 nt, Artificial Sequence
  source: /note=“Description of Artificial Sequence: Synthetic oligonucleotide”
auuuugaauguuuaaaauua

SEQ ID NO: 124 - RNA, 20 nt, Artificial Sequence
  source: /note=“Description of Artificial Sequence: Synthetic oligonucleotide”
aaauuuaaucuggcugaaua

SEQ ID NO: 125 - RNA, 20 nt, Artificial Sequence
  source: /note=“Description of Artificial Sequence: Synthetic oligonucleotide”
gaacuucguuaaauuuaauc

SEQ ID NO: 126 - RNA, 20 nt, Artificial Sequence
  source: /note=“Description of Artificial Sequence: Synthetic oligonucleotide”
auuaaauuuaacgaaguucc

SEQ ID NO: 127 - RNA, 20 nt, Artificial Sequence
  source: /note=“Description of Artificial Sequence: Synthetic oligonucleotide”
uuaaauuuaacgaaguuccu

SEQ ID NO: 128 - RNA, 20 nt, Artificial Sequence
  source: /note=“Description of Artificial Sequence: Synthetic oligonucleotide”
uucuguacuagcauauuccc

SEQ ID NO: 129 - RNA, 20 nt, Artificial Sequence
  source: /note=“Description of Artificial Sequence: Synthetic oligonucleotide”
uguguucuuaaaaaaaaaug

SEQ ID NO: 130 - RNA, 20 nt, Artificial Sequence
  source: /note=“Description of Artificial Sequence: Synthetic oligonucleotide”
aaaaauguggaauuagaccc

SEQ ID NO: 131 - RNA, 20 nt, Artificial Sequence
  source: /note=“Description of Artificial Sequence: Synthetic oligonucleotide”
cuacugggaucuucauuccu

SEQ ID NO: 132 - RNA, 20 nt, Artificial Sequence
  source: /note=“Description of Artificial Sequence: Synthetic oligonucleotide”
acuacugggaucuucauucc

SEQ ID NO: 133 - RNA, 20 nt, Artificial Sequence
  source: /note=“Description of Artificial Sequence: Synthetic oligonucleotide”
gaaaagagugaaaaacuacu

SEQ ID NO: 134 - RNA, 20 nt, Artificial Sequence
  source: /note=“Description of Artificial Sequence: Synthetic oligonucleotide”
agaaaagagugaaaaacuac

SEQ ID NO: 135 - RNA, 20 nt, Artificial Sequence
  source: /note=“Description of Artificial Sequence: Synthetic oligonucleotide”
gaauucaaauaaugccacaa

SEQ ID NO: 136 - RNA, 20 nt, Artificial Sequence
  source: /note=“Description of Artificial Sequence: Synthetic oligonucleotide”
uguguauuugucugccauug

SEQ ID NO: 137 - RNA, 20 nt, Artificial Sequence
  source: /note=“Description of Artificial Sequence: Synthetic oligonucleotide”
cacccaugagcauauccaaa

SEQ ID NO: 138 - RNA, 20 nt, Artificial Sequence
  source: /note=“Description of Artificial Sequence: Synthetic oligonucleotide”
uuccuuuuggauaugcucau

SEQ ID NO: 139 - RNA, 20 nt, Artificial Sequence
  source: /note=“Description of Artificial Sequence: Synthetic oligonucleotide”
cuuccuuuuggauaugcuca

SEQ ID NO: 140 - RNA, 20 nt, Artificial Sequence
  source: /note=“Description of Artificial Sequence: Synthetic oligonucleotide”
caugagcauauccaaaagga

SEQ ID NO: 141 - RNA, 20 nt, Artificial Sequence
  source: /note=“Description of Artificial Sequence: Synthetic oligonucleotide”
uauccaaaaggaaggauuga

SEQ ID NO: 142 - RNA, 20 nt, Artificial Sequence
  source: /note=“Description of Artificial Sequence: Synthetic oligonucleotide”
uuuccuucaauccuuccuuu

SEQ ID NO: 143 - RNA, 20 nt, Artificial Sequence
  source: /note=“Description of Artificial Sequence: Synthetic oligonucleotide”
aaggaaggauugaaggaaag

SEQ ID NO: 144 - RNA, 20 nt, Artificial Sequence
  source: /note=“Description of Artificial Sequence: Synthetic oligonucleotide”
gaaggauugaaggaaagagg

SEQ ID NO: 145 - RNA, 20 nt, Artificial Sequence
  source: /note=“Description of Artificial Sequence: Synthetic oligonucleotide”
gaggaggaagaaauggagaa

SEQ ID NO: 146 - RNA, 20 nt, Artificial Sequence
  source: /note=“Description of Artificial Sequence: Synthetic oligonucleotide”
aggaagaaauggagaaagga

SEQ ID NO: 147 - RNA, 20 nt, Artificial Sequence
  source: /note=“Description of Artificial Sequence: Synthetic oligonucleotide”
gaaggaagaggggaagagag

SEQ ID NO: 148 - RNA, 20 nt, Artificial Sequence
  source: /note=“Description of Artificial Sequence: Synthetic oligonucleotide”
gaagaggggaagagagagga

SEQ ID NO: 149 - RNA, 20 nt, Artificial Sequence
  source: /note=“Description of Artificial Sequence: Synthetic oligonucleotide”
aggggaagagagaggaugga

SEQ ID NO: 150 - RNA, 20 nt, Artificial Sequence
  source: /note=“Description of Artificial Sequence: Synthetic oligonucleotide”
ggggaagagagaggauggaa

SEQ ID NO: 151 - RNA, 20 nt, Artificial Sequence
  source: /note=“Description of Artificial Sequence: Synthetic oligonucleotide”
aagagagaggauggaaggga

SEQ ID NO: 152 - RNA, 20 nt, Artificial Sequence
  source: /note=“Description of Artificial Sequence: Synthetic oligonucleotide”
agagaggauggaagggaugg

SEQ ID NO: 153 - RNA, 20 nt, Artificial Sequence
  source: /note=“Description of Artificial Sequence: Synthetic oligonucleotide”
ggaagggauggaggagaaga

SEQ ID NO: 154 - RNA, 20 nt, Artificial Sequence
  source: /note=“Description of Artificial Sequence: Synthetic oligonucleotide”
gaagaaggaaaaauaaauaa

SEQ ID NO: 155 - RNA, 20 nt, Artificial Sequence
  source: /note=“Description of Artificial Sequence: Synthetic oligonucleotide”
aggaaaaauaaauaauggag

SEQ ID NO: 156 - RNA, 20 nt, Artificial Sequence
  source: /note=“Description of Artificial Sequence: Synthetic oligonucleotide”
aaauaaauaauggagaggag

SEQ ID NO: 157 - RNA, 20 nt, Artificial Sequence
  source: /note=“Description of Artificial Sequence: Synthetic oligonucleotide”
uggagaggagaggagaaaaa

SEQ ID NO: 158 - RNA, 20 nt, Artificial Sequence
  source: /note=“Description of Artificial Sequence: Synthetic oligonucleotide”
agaggagaggagaaaaaagg

SEQ ID NO: 159 - RNA, 20 nt, Artificial Sequence
  source: /note=“Description of Artificial Sequence: Synthetic oligonucleotide”
gaggagaggagaaaaaagga

SEQ ID NO: 160 - RNA, 20 nt, Artificial Sequence
  source: /note=“Description of Artificial Sequence: Synthetic oligonucleotide”
aggagaggagaaaaaaggag

SEQ ID NO: 161 - RNA, 20 nt, Artificial Sequence
  source: /note=“Description of Artificial Sequence: Synthetic oligonucleotide”
aggagaaaaaaggaggggag

SEQ ID NO: 162 - RNA, 20 nt, Artificial Sequence
  source: /note=“Description of Artificial Sequence: Synthetic oligonucleotide”
gagaggagaggagaagggau

SEQ ID NO: 163 - RNA, 20 nt, Artificial Sequence
  source: /note=“Description of Artificial Sequence: Synthetic oligonucleotide”
agaggagaggagaagggaua

SEQ ID NO: 164 - RNA, 20 nt, Artificial Sequence
  source: /note=“Description of Artificial Sequence: Synthetic oligonucleotide”
gaagagaaagagaaagggaa

SEQ ID NO: 165 - RNA, 20 nt, Artificial Sequence
  source: /note=“Description of Artificial Sequence: Synthetic oligonucleotide”
aagagaggaaagaagagaag

SEQ ID NO: 166 - RNA, 20 nt, Artificial Sequence
  source: /note=“Description of Artificial Sequence: Synthetic oligonucleotide”
gagagaaaagaaacgaagag

SEQ ID NO: 167 - RNA, 20 nt, Artificial Sequence
  source: /note=“Description of Artificial Sequence: Synthetic oligonucleotide”
agagaaaagaaacgaagaga

SEQ ID NO: 168 - RNA, 20 nt, Artificial Sequence
  source: /note=“Description of Artificial Sequence: Synthetic oligonucleotide”
gagaaaagaaacgaagagag

SEQ ID NO: 169 - RNA, 20 nt, Artificial Sequence
  source: /note=“Description of Artificial Sequence: Synthetic oligonucleotide”
aaagaaacgaagagagggga

SEQ ID NO: 170 - RNA, 20 nt, Artificial Sequence
  source: /note=“Description of Artificial Sequence: Synthetic oligonucleotide”
aagaaacgaagagaggggaa

SEQ ID NO: 171 - RNA, 20 nt, Artificial Sequence
  source: /note=“Description of Artificial Sequence: Synthetic oligonucleotide”
ggaagggaaggaaaaaaaag

SEQ ID NO: 172 - RNA, 20 nt, Artificial Sequence
  source: /note=“Description of Artificial Sequence: Synthetic oligonucleotide”
aagacugacaguucaaauuu

SEQ ID NO: 173 - RNA, 20 nt, Artificial Sequence
  source: /note=“Description of Artificial Sequence: Synthetic oligonucleotide”
acugacaguucaaauuuugg

SEQ ID NO: 174 - RNA, 20 nt, Artificial Sequence
  source: /note=“Description of Artificial Sequence: Synthetic oligonucleotide”
uucaaauuuugguggugaua

SEQ ID NO: 175 - RNA, 20 nt, Artificial Sequence
  source: /note=“Description of Artificial Sequence: Synthetic oligonucleotide”
aauagaaacucaaacucugu

SEQ ID NO: 176 - RNA, 20 nt, Artificial Sequence
  source: /note=“Description of Artificial Sequence: Synthetic oligonucleotide”
guacaauaguauaaccccuu

SEQ ID NO: 177 - RNA, 20 nt, Artificial Sequence
  source: /note=“Description of Artificial Sequence: Synthetic oligonucleotide”
cuauuaaagguuuuccaaag

SEQ ID NO: 178 - RNA, 20 nt, Artificial Sequence
  source: /note=“Description of Artificial Sequence: Synthetic oligonucleotide”
acuauuaaagguuuuccaaa

SEQ ID NO: 179 - RNA, 20 nt, Artificial Sequence
  source: /note=“Description of Artificial Sequence: Synthetic oligonucleotide”
uacuauuaaagguuuuccaa

SEQ ID NO: 180 - RNA, 20 nt, Artificial Sequence
  source: /note=“Description of Artificial Sequence: Synthetic oligonucleotide”
gcauuuguggauacuauuaa

SEQ ID NO: 181 - RNA, 20 nt, Artificial Sequence
  source: /note=“Description of Artificial Sequence: Synthetic oligonucleotide”
uuaauaguauccacaaaugc

SEQ ID NO: 182 - RNA, 20 nt, Artificial Sequence
  source: /note=“Description of Artificial Sequence: Synthetic oligonucleotide”
uaugcaauuuuugccaagau

SEQ ID NO: 183 - RNA, 20 nt, Artificial Sequence
  source: /note=“Description of Artificial Sequence: Synthetic oligonucleotide”
uuuugccaagaugggaguau

SEQ ID NO: 184 - RNA, 20 nt, Artificial Sequence
  source: /note=“Description of Artificial Sequence: Synthetic oligonucleotide”
uuugccaagaugggaguaug

SEQ ID NO: 185 - RNA, 20 nt, Artificial Sequence
  source: /note=“Description of Artificial Sequence: Synthetic oligonucleotide”
ucagugagaugagauaucaa

SEQ ID NO: 186 - RNA, 20 nt, Artificial Sequence
  source: /note=“Description of Artificial Sequence: Synthetic oligonucleotide”
cagugagaugagauaucaaa

SEQ ID NO: 187 - RNA, 20 nt, Artificial Sequence
  source: /note=“Description of Artificial Sequence: Synthetic oligonucleotide”
ccaucucccuaaucuccaau

SEQ ID NO: 188 - RNA, 20 nt, Artificial Sequence
  source: /note=“Description of Artificial Sequence: Synthetic oligonucleotide”
ccaauuggagauuagggaga

SEQ ID NO: 189 - RNA, 20 nt, Artificial Sequence
  source: /note=“Description of Artificial Sequence: Synthetic oligonucleotide”
gcuuugccaauuggagauua

SEQ ID NO: 190 - RNA, 20 nt, Artificial Sequence
  source: /note=“Description of Artificial Sequence: Synthetic oligonucleotide”
ggcuuugccaauuggagauu

SEQ ID NO: 191 - RNA, 20 nt, Artificial Sequence
  source: /note=“Description of Artificial Sequence: Synthetic oligonucleotide”
aauuggcaaagccagacuug

SEQ ID NO: 192 - RNA, 20 nt, Artificial Sequence
  source: /note=“Description of Artificial Sequence: Synthetic oligonucleotide”
agucuguauugccccaaguc

SEQ ID NO: 193 - RNA, 20 nt, Artificial Sequence
  source: /note=“Description of Artificial Sequence: Synthetic oligonucleotide”
agacuuggggcaauacagac

SEQ ID NO: 194 - RNA, 20 nt, Artificial Sequence
  source: /note=“Description of Artificial Sequence: Synthetic oligonucleotide”
acauuuggugauaaaucauu

SEQ ID NO: 195 - RNA, 20 nt, Artificial Sequence
  source: /note=“Description of Artificial Sequence: Synthetic oligonucleotide”
ccaaauguucuuucuucagc

SEQ ID NO: 196 - RNA, 20 nt, Artificial Sequence
  source: /note=“Description of Artificial Sequence: Synthetic oligonucleotide”
auaauaguauaugcuucaua

SEQ ID NO: 197 - RNA, 20 nt, Artificial Sequence
  source: /note=“Description of Artificial Sequence: Synthetic oligonucleotide”
cggagcacuuacucugcucu

SEQ ID NO: 198 - RNA, 20 nt, Artificial Sequence
  source: /note=“Description of Artificial Sequence: Synthetic oligonucleotide”
agcauuuuaguucacaagcu

SEQ ID NO: 199 - RNA, 20 nt, Artificial Sequence
  source: /note=“Description of Artificial Sequence: Synthetic oligonucleotide”
uguaacuaauaaauaccagg

SEQ ID NO: 200 - RNA, 20 nt, Artificial Sequence
  source: /note=“Description of Artificial Sequence: Synthetic oligonucleotide”
agguguaacuaauaaauacc

SEQ ID NO: 201 - RNA, 20 nt, Artificial Sequence
  source: /note=“Description of Artificial Sequence: Synthetic oligonucleotide”
ucugacccaaacuaggaauu

SEQ ID NO: 202 - RNA, 20 nt, Artificial Sequence
  source: /note=“Description of Artificial Sequence: Synthetic oligonucleotide”
aaggaaaagaauaugacguc

SEQ ID NO: 203 - RNA, 20 nt, Artificial Sequence
  source: /note=“Description of Artificial Sequence: Synthetic oligonucleotide”
aggaaaagaauaugacguca

SEQ ID NO: 204 - RNA, 20 nt, Artificial Sequence
  source: /note=“Description of Artificial Sequence: Synthetic oligonucleotide”
ggaaaagaauaugacgucag

SEQ ID NO: 205 - RNA, 20 nt, Artificial Sequence
  source: /note=“Description of Artificial Sequence: Synthetic oligonucleotide”
gaaaagaauaugacgucagg

SEQ ID NO: 206 - RNA, 20 nt, Artificial Sequence
  source: /note=“Description of Artificial Sequence: Synthetic oligonucleotide”
ucagggggaggcaagucagu

SEQ ID NO: 207 - RNA, 20 nt, Artificial Sequence
  source: /note=“Description of Artificial Sequence: Synthetic oligonucleotide”
cagggggaggcaagucaguu

SEQ ID NO: 208 - RNA, 20 nt, Artificial Sequence
  source: /note=“Description of Artificial Sequence: Synthetic oligonucleotide”
aucacauauaggcaccuauc

SEQ ID NO: 209 - RNA, 20 nt, Artificial Sequence
  source: /note=“Description of Artificial Sequence: Synthetic oligonucleotide”
cagguaccagcuacuguguu

SEQ ID NO: 210 - RNA, 20 nt, Artificial Sequence
  source: /note=“Description of Artificial Sequence: Synthetic oligonucleotide”
acuauccacggauauacacu

SEQ ID NO: 211 - RNA, 20 nt, Artificial Sequence
  source: /note=“Description of Artificial Sequence: Synthetic oligonucleotide”
cacaguagcugguaccugau

SEQ ID NO: 212 - RNA, 20 nt, Artificial Sequence
  source: /note=“Description of Artificial Sequence: Synthetic oligonucleotide”
aucacauauaggcaccuauc

SEQ ID NO: 213 - RNA, 20 nt, Artificial Sequence
  source: /note=“Description of Artificial Sequence: Synthetic oligonucleotide”
ugauaggugccuauauguga

SEQ ID NO: 214 - RNA, 20 nt, Artificial Sequence
  source: /note=“Description of Artificial Sequence: Synthetic oligonucleotide”
aggugccuauaugugaugga

SEQ ID NO: 215 - RNA, 20 nt, Artificial Sequence
  source: /note=“Description of Artificial Sequence: Synthetic oligonucleotide”
ggugccuauaugugauggau

SEQ ID NO: 216 - RNA, 20 nt, Artificial Sequence
  source: /note=“Description of Artificial Sequence: Synthetic oligonucleotide”
uccacccauccaucacauau

SEQ ID NO: 217 - RNA, 20 nt, Artificial Sequence
  source: /note=“Description of Artificial Sequence: Synthetic oligonucleotide”
acagcccgacagaugaaaaa

SEQ ID NO: 218 - RNA, 20 nt, Artificial Sequence
  source: /note=“Description of Artificial Sequence: Synthetic oligonucleotide”
augaaaaauggacaauuaug

SEQ ID NO: 219 - RNA, 20 nt, Artificial Sequence
  source: /note=“Description of Artificial Sequence: Synthetic oligonucleotide”
aaaaauggacaauuaugagg

SEQ ID NO: 220 - RNA, 20 nt, Artificial Sequence
  source: /note=“Description of Artificial Sequence: Synthetic oligonucleotide”
aaaauggacaauuaugagga

SEQ ID NO: 221 - RNA, 20 nt, Artificial Sequence
  source: /note=“Description of Artificial Sequence: Synthetic oligonucleotide”
aaauggacaauuaugaggag

SEQ ID NO: 222 - RNA, 20 nt, Artificial Sequence
  source: /note=“Description of Artificial Sequence: Synthetic oligonucleotide”
gaggaggggagagugcagac

SEQ ID NO: 223 - RNA, 20 nt, Artificial Sequence
  source: /note=“Description of Artificial Sequence: Synthetic oligonucleotide”
aggaggggagagugcagaca

SEQ ID NO: 224 - RNA, 20 nt, Artificial Sequence
  source: /note=“Description of Artificial Sequence: Synthetic oligonucleotide”
cuucaccuccuuuacaauuu

SEQ ID NO: 225 - RNA, 20 nt, Artificial Sequence
  source: /note=“Description of Artificial Sequence: Synthetic oligonucleotide”
uucaccuccuuuacaauuuu

SEQ ID NO: 226 - RNA, 20 nt, Artificial Sequence
  source: /note=“Description of Artificial Sequence: Synthetic oligonucleotide”
gacucccaaaauuguaaagg

SEQ ID NO: 227 - RNA, 20 nt, Artificial Sequence
  source: /note=“Description of Artificial Sequence: Synthetic oligonucleotide”
guggacucccaaaauuguaa

SEQ ID NO: 228 - RNA, 20 nt, Artificial Sequence
  source: /note=“Description of Artificial Sequence: Synthetic oligonucleotide”
uuuugggaguccacacggca

SEQ ID NO: 229 - RNA, 20 nt, Artificial Sequence
  source: /note=“Description of Artificial Sequence: Synthetic oligonucleotide”
aauuuguaugccaugccgug

SEQ ID NO: 230 - RNA, 20 nt, Artificial Sequence
  source: /note=“Description of Artificial Sequence: Synthetic oligonucleotide”
ccaagagagccuuccgaaag

SEQ ID NO: 231 - RNA, 20 nt, Artificial Sequence
  source: /note=“Description of Artificial Sequence: Synthetic oligonucleotide”
ccaggggggccucuuucgga

SEQ ID NO: 232 - RNA, 20 nt, Artificial Sequence
  source: /note=“Description of Artificial Sequence: Synthetic oligonucleotide”
ccuuccgaaagaggcccccc

SEQ ID NO: 233 - RNA, 20 nt, Artificial Sequence
  source: /note=“Description of Artificial Sequence: Synthetic oligonucleotide”
cuuccgaaagaggccccccu

SEQ ID NO: 234 - RNA, 20 nt, Artificial Sequence
  source: /note=“Description of Artificial Sequence: Synthetic oligonucleotide”
aagaggccccccugggcaaa

SEQ ID NO: 235 - RNA, 20 nt, Artificial Sequence
  source: /note=“Description of Artificial Sequence: Synthetic oligonucleotide”
cgguggccguuugcccaggg

SEQ ID NO: 236 - RNA, 20 nt, Artificial Sequence
  source: /note=“Description of Artificial Sequence: Synthetic oligonucleotide”
ucgguggccguuugcccagg

SEQ ID NO: 237 - RNA, 20 nt, Artificial Sequence
  source: /note=“Description of Artificial Sequence: Synthetic oligonucleotide”
aucgguggccguuugcccag

SEQ ID NO: 238 - RNA, 20 nt, Artificial Sequence
  source: /note=“Description of Artificial Sequence: Synthetic oligonucleotide”
caucgguggccguuugccca

SEQ ID NO: 239 - RNA, 20 nt, Artificial Sequence
  source: /note=“Description of Artificial Sequence: Synthetic oligonucleotide”
ccugggcaaacggccaccga

SEQ ID NO: 240 - RNA, 20 nt, Artificial Sequence
  source: /note=“Description of Artificial Sequence: Synthetic oligonucleotide”
ccaucgguggccguuugccc

SEQ ID NO: 241 - RNA, 20 nt, Artificial Sequence
  source: /note=“Description of Artificial Sequence: Synthetic oligonucleotide”
gcaaacggccaccgauggag

SEQ ID NO: 242 - RNA, 20 nt, Artificial Sequence
  source: /note=“Description of Artificial Sequence: Synthetic oligonucleotide”
cuggcagaccucuccaucgg

SEQ ID NO: 243 - RNA, 20 nt, Artificial Sequence
  source: /note=“Description of Artificial Sequence: Synthetic oligonucleotide”
ggacuggcagaccucuccau

SEQ ID NO: 244 - RNA, 20 nt, Artificial Sequence
  source: /note=“Description of Artificial Sequence: Synthetic oligonucleotide”
ucugauuagggugggggcgu

SEQ ID NO: 245 - RNA, 20 nt, Artificial Sequence
  source: /note=“Description of Artificial Sequence: Synthetic oligonucleotide”
cacgcccccacccuaaucag

SEQ ID NO: 246 - RNA, 20 nt, Artificial Sequence
  source: /note=“Description of Artificial Sequence: Synthetic oligonucleotide”
uuggccucugauuagggugg

SEQ ID NO: 247 - RNA, 20 nt, Artificial Sequence
  source: /note=“Description of Artificial Sequence: Synthetic oligonucleotide”
uuuggccucugauuagggug

SEQ ID NO: 248 - RNA, 20 nt, Artificial Sequence
  source: /note=“Description of Artificial Sequence: Synthetic oligonucleotide”
guuuggccucugauuagggu

SEQ ID NO: 249 - RNA, 20 nt, Artificial Sequence
  source: /note=“Description of Artificial Sequence: Synthetic oligonucleotide”
gguuuggccucugauuaggg

SEQ ID NO: 250 - RNA, 20 nt, Artificial Sequence
  source: /note=“Description of Artificial Sequence: Synthetic oligonucleotide”
aaggguuuggccucugauua

SEQ ID NO: 251 - RNA, 20 nt, Artificial Sequence
  source: /note=“Description of Artificial Sequence: Synthetic oligonucleotide”
gaaggguuuggccucugauu

SEQ ID NO: 252 - RNA, 20 nt, Artificial Sequence
  source: /note=“Description of Artificial Sequence: Synthetic oligonucleotide”
uugcuuuuaucacaggcucc

SEQ ID NO: 253 - RNA, 20 nt, Artificial Sequence
  source: /note=“Description of Artificial Sequence: Synthetic oligonucleotide”
cuaacaguugcuuuuaucac

SEQ ID NO: 254 - RNA, 20 nt, Artificial Sequence
  source: /note=“Description of Artificial Sequence: Synthetic oligonucleotide”
cuucaaaguuguauugaccc

SEQ ID NO: 255 - RNA, 20 nt, Artificial Sequence
  source: /note=“Description of Artificial Sequence: Synthetic oligonucleotide”
acucuuagacauaacacacc

SEQ ID NO: 256 - RNA, 20 nt, Artificial Sequence
  source: /note=“Description of Artificial Sequence: Synthetic oligonucleotide”
uagaugccauaucucuuuuc

SEQ ID NO: 257 - RNA, 20 nt, Artificial Sequence
  source: /note=“Description of Artificial Sequence: Synthetic oligonucleotide”
cauaggccagaaaagagaua

SEQ ID NO: 258 - RNA, 20 nt, Artificial Sequence
  source: /note=“Description of Artificial Sequence: Synthetic oligonucleotide”
ggccuauguuauuaccugua

SEQ ID NO: 259 - RNA, 20 nt, Artificial Sequence
  source: /note=“Description of Artificial Sequence: Synthetic oligonucleotide”
guccauacagguaauaacau

SEQ ID NO: 260 - RNA, 20 nt, Artificial Sequence
  source: /note=“Description of Artificial Sequence: Synthetic oligonucleotide”
uaccuguauggacuuugcac

SEQ ID NO: 261 - RNA, 20 nt, Artificial Sequence
  source: /note=“Description of Artificial Sequence: Synthetic oligonucleotide”
uuccagugcaaaguccauac

SEQ ID NO: 262 - RNA, 20 nt, Artificial Sequence
  source: /note=“Description of Artificial Sequence: Synthetic oligonucleotide”
ugcucuuacuuaugcacacc

SEQ ID NO: 263 - RNA, 20 nt, Artificial Sequence
  source: /note=“Description of Artificial Sequence: Synthetic oligonucleotide”
gcucuuacuuaugcacaccu

SEQ ID NO: 264 - RNA, 20 nt, Artificial Sequence
  source: /note=“Description of Artificial Sequence: Synthetic oligonucleotide”
cucuuacuuaugcacaccug

SEQ ID NO: 265 - RNA, 20 nt, Artificial Sequence
  source: /note=“Description of Artificial Sequence: Synthetic oligonucleotide”
cagggcuggcucuaugcccc

SEQ ID NO: 266 - RNA, 20 nt, Artificial Sequence
  source: /note=“Description of Artificial Sequence: Synthetic oligonucleotide”
gcugaaaagcgauacagggc

SEQ ID NO: 267 - RNA, 20 nt, Artificial Sequence
  source: /note=“Description of Artificial Sequence: Synthetic oligonucleotide”
gauggcugaaaagcgauaca

SEQ ID NO: 268 - RNA, 20 nt, Artificial Sequence
  source: /note=“Description of Artificial Sequence: Synthetic oligonucleotide”
agauggcugaaaagcgauac

SEQ ID NO: 269 - RNA, 20 nt, Artificial Sequence
  source: /note=“Description of Artificial Sequence: Synthetic oligonucleotide”
gggaguuaucuguagugaga

SEQ ID NO: 270 - RNA, 20 nt, Artificial Sequence
  source: /note=“Description of Artificial Sequence: Synthetic oligonucleotide”
aaggcagcuagacaggacuu

SEQ ID NO: 271 - RNA, 20 nt, Artificial Sequence
  source: /note=“Description of Artificial Sequence: Synthetic oligonucleotide”
gaaggcagcuagacaggacu

SEQ ID NO: 272 - RNA, 20 nt, Artificial Sequence
  source: /note=“Description of Artificial Sequence: Synthetic oligonucleotide”
gauaaggaaggcagcuagac

SEQ ID NO: 273 - RNA, 20 nt, Artificial Sequence
  source: /note=“Description of Artificial Sequence: Synthetic oligonucleotide”
cuagcugccuuccuuaucac

SEQ ID NO: 274 - RNA, 20 nt, Artificial Sequence
  source: /note=“Description of Artificial Sequence: Synthetic oligonucleotide”
ugggugcuauuccugugaua

SEQ ID NO: 275 - RNA, 20 nt, Artificial Sequence
  source: /note=“Description of Artificial Sequence: Synthetic oligonucleotide”
uaucacaggaauagcaccca

SEQ ID NO: 276 - RNA, 20 nt, Artificial Sequence
  source: /note=“Description of Artificial Sequence: Synthetic oligonucleotide”
cugagguacugauggaccuu

SEQ ID NO: 277 - RNA, 20 nt, Artificial Sequence
  source: /note=“Description of Artificial Sequence: Synthetic oligonucleotide”
ucugagguacugauggaccu

SEQ ID NO: 278-333 - skipped (56 entries)

SEQ ID NO: 334 - RNA, 20 nt, Artificial Sequence
  source: /note=“Description of Artificial Sequence: Synthetic oligonucleotide”
uuagggugggggcgugggug

SEQ ID NO: 335 - RNA, 20 nt, Artificial Sequence
  source: /note=“Description of Artificial Sequence: Synthetic oligonucleotide”
uuuuaucacaggcuccagga

SEQ ID NO: 336 - RNA, 20 nt, Artificial Sequence
  source: /note=“Description of Artificial Sequence: Synthetic oligonucleotide”
uuuaucacaggcuccaggaa

SEQ ID NO: 337 - RNA, 20 nt, Artificial Sequence
  source: /note=“Description of Artificial Sequence: Synthetic oligonucleotide”
cacaggcuccaggaaggguu

SEQ ID NO: 338 - RNA, 20 nt, Artificial Sequence
  source: /note=“Description of Artificial Sequence: Synthetic oligonucleotide”
aucagaggccaaacccuucc

SEQ ID NO: 339 - RNA, 20 nt, Artificial Sequence
  source: /note=“Description of Artificial Sequence: Synthetic oligonucleotide”
cucugauuagggugggggcg

SEQ ID NO: 340 - RNA, 20 nt, Artificial Sequence
  source: /note=“Description of Artificial Sequence: Synthetic oligonucleotide”
gauuagggugggggcguggg

SEQ ID NO: 341 - RNA, 20 nt, Artificial Sequence
  source: /note=“Description of Artificial Sequence: Synthetic oligonucleotide”
auuagggugggggcgugggu

SEQ ID NO: 342 - RNA, 20 nt, Artificial Sequence
  source: /note=“Description of Artificial Sequence: Synthetic oligonucleotide”
uaucaagcauccagcauuug

SEQ ID NO: 343 - RNA, 20 nt, Artificial Sequence
  source: /note=“Description of Artificial Sequence: Synthetic oligonucleotide”
uaucuaaaaauguaauugcu

SEQ ID NO: 344 - RNA, 20 nt, Artificial Sequence
  source: /note=“Description of Artificial Sequence: Synthetic oligonucleotide”
agcauuucuauacaugucuu

SEQ ID NO: 345 - RNA, 20 nt, Artificial Sequence
  source: /note=“Description of Artificial Sequence: Synthetic oligonucleotide”
uaaucauaaaaaccucaaac

SEQ ID NO: 346 - RNA, 20 nt, Artificial Sequence
  source: /note=“Description of Artificial Sequence: Synthetic oligonucleotide”
uuuaaguggcuaccgguuug

SEQ ID NO: 347 - RNA, 20 nt, Artificial Sequence
  source: /note=“Description of Artificial Sequence: Synthetic oligonucleotide”
guaagcauuuaaguggcuac

SEQ ID NO: 348 - RNA, 20 nt, Artificial Sequence
  source: /note=“Description of Artificial Sequence: Synthetic oligonucleotide”
acuguugguaagcauuuaag

SEQ ID NO: 349 - RNA, 20 nt, Artificial Sequence
  source: /note=“Description of Artificial Sequence: Synthetic oligonucleotide”
uaauuuaucaauucuacugu

SEQ ID NO: 350 - RNA, 20 nt, Artificial Sequence
  source: /note=“Description of Artificial Sequence: Synthetic oligonucleotide”
acaguagaauugauaaauua

SEQ ID NO: 351 - RNA, 20 nt, Artificial Sequence
  source: /note=“Description of Artificial Sequence: Synthetic oligonucleotide”
caaaugcauuuuacagcauu

SEQ ID NO: 352 - RNA, 20 nt, Artificial Sequence
  source: /note=“Description of Artificial Sequence: Synthetic oligonucleotide”
gguugauuaaaaguaaccag

SEQ ID NO: 353 - RNA, 20 nt, Artificial Sequence
  source: /note=“Description of Artificial Sequence: Synthetic oligonucleotide”
auauaguuugaacucaccuc

SEQ ID NO: 354 - RNA, 20 nt, Artificial Sequence
  source: /note=“Description of Artificial Sequence: Synthetic oligonucleotide”
uuuauuuguauauagaaaga

SEQ ID NO: 355 - RNA, 20 nt, Artificial Sequence
  source: /note=“Description of Artificial Sequence: Synthetic oligonucleotide”
ugccugagauucugaucaca

SEQ ID NO: 356 - RNA, 20 nt, Artificial Sequence
  source: /note=“Description of Artificial Sequence: Synthetic oligonucleotide”
gccugagauucugaucacaa

SEQ ID NO: 357 - RNA, 20 nt, Artificial Sequence
  source: /note=“Description of Artificial Sequence: Synthetic oligonucleotide”
ccugagauucugaucacaag

SEQ ID NO: 358 - RNA, 20 nt, Artificial Sequence
  source: /note=“Description of Artificial Sequence: Synthetic oligonucleotide”
ccccuugugaucagaaucuc

SEQ ID NO: 359 - RNA, 20 nt, Artificial Sequence
  source: /note=“Description of Artificial Sequence: Synthetic oligonucleotide”
aaggggaaauguuauaaaau

SEQ ID NO: 360 - RNA, 20 nt, Artificial Sequence
  source: /note=“Description of Artificial Sequence: Synthetic oligonucleotide”
aggggaaauguuauaaaaua

SEQ ID NO: 361 - RNA, 20 nt, Artificial Sequence
  source: /note=“Description of Artificial Sequence: Synthetic oligonucleotide”
uguuauaaaauaggguagag

SEQ ID NO: 362 - RNA, 20 nt, Artificial Sequence
  source: /note=“Description of Artificial Sequence: Synthetic oligonucleotide”
caaaguuuaaaggucauuca

SEQ ID NO: 363 - RNA, 20 nt, Artificial Sequence
  source: /note=“Description of Artificial Sequence: Synthetic oligonucleotide”
uaacuuguaacaaaguuuaa

SEQ ID NO: 364 - RNA, 20 nt, Artificial Sequence
  source: /note=“Description of Artificial Sequence: Synthetic oligonucleotide”
caaguuauuuuucuguaacc

SEQ ID NO: 365 - RNA, 20 nt, Artificial Sequence
  source: /note=“Description of Artificial Sequence: Synthetic oligonucleotide”
aauaucuuucguuggcuucc

SEQ ID NO: 366 - RNA, 20 nt, Artificial Sequence
  source: /note=“Description of Artificial Sequence: Synthetic oligonucleotide”
aauuauucaauaucuuucgu

SEQ ID NO: 367 - RNA, 20 nt, Artificial Sequence
  source: /note=“Description of Artificial Sequence: Synthetic oligonucleotide”
gauauugaauaauucaagaa

SEQ ID NO: 368 - RNA, 20 nt, Artificial Sequence
  source: /note=“Description of Artificial Sequence: Synthetic oligonucleotide”
auugaauaauucaagaaagg

SEQ ID NO: 369 - RNA, 20 nt, Artificial Sequence
  source: /note=“Description of Artificial Sequence: Synthetic oligonucleotide”
gaauaauucaagaaaggugg

SEQ ID NO: 370 - RNA, 20 nt, Artificial Sequence
  source: /note=“Description of Artificial Sequence: Synthetic oligonucleotide”
auucaagaaaggugguggca

SEQ ID NO: 371 - RNA, 20 nt, Artificial Sequence
  source: /note=“Description of Artificial Sequence: Synthetic oligonucleotide”
uauuuuagaaguagagaaaa

SEQ ID NO: 372 - RNA, 20 nt, Artificial Sequence
  source: /note=“Description of Artificial Sequence: Synthetic oligonucleotide”
auuuuagaaguagagaaaau

SEQ ID NO: 373 - RNA, 20 nt, Artificial Sequence
  source: /note=“Description of Artificial Sequence: Synthetic oligonucleotide”
gaaaaugggagacaaauagc

SEQ ID NO: 374 - RNA, 20 nt, Artificial Sequence
  source: /note=“Description of Artificial Sequence: Synthetic oligonucleotide”
aaaaugggagacaaauagcu

SEQ ID NO: 375 - RNA, 20 nt, Artificial Sequence
  source: /note=“Description of Artificial Sequence: Synthetic oligonucleotide”
agcugggcuucuguugcagu

SEQ ID NO: 376 - RNA, 20 nt, Artificial Sequence
  source: /note=“Description of Artificial Sequence: Synthetic oligonucleotide”
gcugggcuucuguugcagua

SEQ ID NO: 377 - RNA, 20 nt, Artificial Sequence
  source: /note=“Description of Artificial Sequence: Synthetic oligonucleotide”
gccauuucuauuaucagacu

SEQ ID NO: 378 - RNA, 20 nt, Artificial Sequence
  source: /note=“Description of Artificial Sequence: Synthetic oligonucleotide”
uccaagucugauaauagaaa

SEQ ID NO: 379 - RNA, 20 nt, Artificial Sequence
  source: /note=“Description of Artificial Sequence: Synthetic oligonucleotide”
uuaucagacuuggaccauga

SEQ ID NO: 380 - RNA, 20 nt, Artificial Sequence
  source: /note=“Description of Artificial Sequence: Synthetic oligonucleotide”
cacgacugacaucaccguca

SEQ ID NO: 381 - RNA, 20 nt, Artificial Sequence
  source: /note=“Description of Artificial Sequence: Synthetic oligonucleotide”
ucagucgugaacacaagaau

SEQ ID NO: 382 - RNA, 20 nt, Artificial Sequence
  source: /note=“Description of Artificial Sequence: Synthetic oligonucleotide”
cagucgugaacacaagaaua

SEQ ID NO: 383 - RNA, 20 nt, Artificial Sequence
  source: /note=“Description of Artificial Sequence: Synthetic oligonucleotide”
ggccacauuugugaguuuag

SEQ ID NO: 384 - RNA, 20 nt, Artificial Sequence
  source: /note=“Description of Artificial Sequence: Synthetic oligonucleotide”
uaccacuaaacucacaaaug

SEQ ID NO: 385 - RNA, 20 nt, Artificial Sequence
  source: /note=“Description of Artificial Sequence: Synthetic oligonucleotide”
uaaaaucagaaauacagucu

SEQ ID NO: 386 - RNA, 20 nt, Artificial Sequence
  source: /note=“Description of Artificial Sequence: Synthetic oligonucleotide”
aaaagauguacuuagauaug

SEQ ID NO: 387 - RNA, 20 nt, Artificial Sequence
  source: /note=“Description of Artificial Sequence: Synthetic oligonucleotide”
uguacuuagauauguggauc

SEQ ID NO: 388 - RNA, 20 nt, Artificial Sequence
  source: /note=“Description of Artificial Sequence: Synthetic oligonucleotide”
agcucagaaagaauacaacc

SEQ ID NO: 389 - RNA, 20 nt, Artificial Sequence
  source: /note=“Description of Artificial Sequence: Synthetic oligonucleotide”
accaggucaagaauacagaa

SEQ ID NO: 390 - RNA, 20 nt, Artificial Sequence
  source: /note=“Description of Artificial Sequence: Synthetic oligonucleotide”
uccauucuguauucuugacc

SEQ ID NO: 391 - RNA, 20 nt, Artificial Sequence
  source: /note=“Description of Artificial Sequence: Synthetic oligonucleotide”
cugucauuuuuaacagguag

SEQ ID NO: 392 - RNA, 20 nt, Artificial Sequence
  source: /note=“Description of Artificial Sequence: Synthetic oligonucleotide”
caucaucugucauuuuuaac

SEQ ID NO: 393 - RNA, 20 nt, Artificial Sequence
  source: /note=“Description of Artificial Sequence: Synthetic oligonucleotide”
aaacacauucuaagauuuua

SEQ ID NO: 394 - RNA, 20 nt, Artificial Sequence
  source: /note=“Description of Artificial Sequence: Synthetic oligonucleotide”
aaucuuagaauguguuugug

SEQ ID NO: 395 - RNA, 20 nt, Artificial Sequence
  source: /note=“Description of Artificial Sequence: Synthetic oligonucleotide”
aucuuagaauguguuuguga

SEQ ID NO: 396 - RNA, 20 nt, Artificial Sequence
  source: /note=“Description of Artificial Sequence: Synthetic oligonucleotide”
uuagaauguguuugugaggg

SEQ ID NO: 397 - RNA, 20 nt, Artificial Sequence
  source: /note=“Description of Artificial Sequence: Synthetic oligonucleotide”
caauuuucuuauauaugaau

SEQ ID NO: 398 - RNA, 20 nt, Artificial Sequence
  source: /note=“Description of Artificial Sequence: Synthetic oligonucleotide”
uugauucuaaaaaaaauguu

SEQ ID NO: 399 - RNA, 20 nt, Artificial Sequence
  source: /note=“Description of Artificial Sequence: Synthetic oligonucleotide”
aaauguuagguaaauucuua

SEQ ID NO: 400 - RNA, 20 nt, Artificial Sequence
  source: /note=“Description of Artificial Sequence: Synthetic oligonucleotide”
gguaaauucuuaaggccaug

SEQ ID NO: 401 - RNA, 20 nt, Artificial Sequence
  source: /note=“Description of Artificial Sequence: Synthetic oligonucleotide”
agaucaaauaacaguccuca

SEQ ID NO: 402 - RNA, 20 nt, Artificial Sequence
  source: /note=“Description of Artificial Sequence: Synthetic oligonucleotide”
gucuguuaauuccaaagacu

SEQ ID NO: 403 - RNA, 20 nt, Artificial Sequence
  source: /note=“Description of Artificial Sequence: Synthetic oligonucleotide”
aaagugaaaagccaagucuu

SEQ ID NO: 404 - RNA, 20 nt, Artificial Sequence
  source: /note=“Description of Artificial Sequence: Synthetic oligonucleotide”
ccugaaaugauuuuacacau

SEQ ID NO: 405 - RNA, 20 nt, Artificial Sequence
  source: /note=“Description of Artificial Sequence: Synthetic oligonucleotide”
ccaauguguaaaaucauuuc

SEQ ID NO: 406 - RNA, 20 nt, Artificial Sequence
  source: /note=“Description of Artificial Sequence: Synthetic oligonucleotide”
cugaaaugauuuuacacauu

SEQ ID NO: 407 - RNA, 20 nt, Artificial Sequence
  source: /note=“Description of Artificial Sequence: Synthetic oligonucleotide”
auuuuacacauugggagauc

SEQ ID NO: 408 - RNA, 20 nt, Artificial Sequence
  source: /note=“Description of Artificial Sequence: Synthetic oligonucleotide”
gguuacauguuuauucuaua

SEQ ID NO: 409 - RNA, 20 nt, Artificial Sequence
  source: /note=“Description of Artificial Sequence: Synthetic oligonucleotide”
ucuauauggauugcauugag

SEQ ID NO: 410 - RNA, 20 nt, Artificial Sequence
  source: /note=“Description of Artificial Sequence: Synthetic oligonucleotide”
aggauuuguauaacagaaua

SEQ ID NO: 411 - RNA, 20 nt, Artificial Sequence
  source: /note=“Description of Artificial Sequence: Synthetic oligonucleotide”
uuuucuuuucucuucugaga

SEQ ID NO: 412 - RNA, 20 nt, Artificial Sequence
  source: /note=“Description of Artificial Sequence: Synthetic oligonucleotide”
gcacucuagcuugggcaaua

SEQ ID NO: 413 - RNA, 20 nt, Artificial Sequence
  source: /note=“Description of Artificial Sequence: Synthetic oligonucleotide”
ugcacucuagcuugggcaau

SEQ ID NO: 414 - RNA, 20 nt, Artificial Sequence
  source: /note=“Description of Artificial Sequence: Synthetic oligonucleotide”
ugcaccauugcacucuagcu

SEQ ID NO: 415 - RNA, 20 nt, Artificial Sequence
  source: /note=“Description of Artificial Sequence: Synthetic oligonucleotide”
gcuauucagguggcugaggc

SEQ ID NO: 416 - RNA, 20 nt, Artificial Sequence
  source: /note=“Description of Artificial Sequence: Synthetic oligonucleotide”
accugaauagcugggacugc

SEQ ID NO: 417 - RNA, 20 nt, Artificial Sequence
  source: /note=“Description of Artificial Sequence: Synthetic oligonucleotide”
gcaggcaugcaccacacgcc

SEQ ID NO: 418 - RNA, 20 nt, Artificial Sequence
  source: /note=“Description of Artificial Sequence: Synthetic oligonucleotide”
uacaaaaucagccgggcgug

SEQ ID NO: 419 - RNA, 20 nt, Artificial Sequence
  source: /note=“Description of Artificial Sequence: Synthetic oligonucleotide”
ggcuuguaaacccagcacuu

SEQ ID NO: 420 - RNA, 20 nt, Artificial Sequence
  source: /note=“Description of Artificial Sequence: Synthetic oligonucleotide”
cuggcuggaugcgguggcuc

SEQ ID NO: 421 - RNA, 20 nt, Artificial Sequence
  source: /note=“Description of Artificial Sequence: Synthetic oligonucleotide”
cugagccaccgcauccagcc

SEQ ID NO: 422 - RNA, 20 nt, Artificial Sequence
  source: /note=“Description of Artificial Sequence: Synthetic oligonucleotide”
cuuauccuggcuggaugcgg

SEQ ID NO: 423 - RNA, 20 nt, Artificial Sequence
  source: /note=“Description of Artificial Sequence: Synthetic oligonucleotide”
caccgcauccagccaggaua

SEQ ID NO: 424 - RNA, 20 nt, Artificial Sequence
  source: /note=“Description of Artificial Sequence: Synthetic oligonucleotide”
gaccuuauccuggcuggaug

SEQ ID NO: 425 - RNA, 20 nt, Artificial Sequence
  source: /note=“Description of Artificial Sequence: Synthetic oligonucleotide”
cuuuuagaccuuauccuggc

SEQ ID NO: 426 - RNA, 20 nt, Artificial Sequence
  source: /note=“Description of Artificial Sequence: Synthetic oligonucleotide”
gccaggauaaggucuaaaag

SEQ ID NO: 427 - RNA, 20 nt, Artificial Sequence
  source: /note=“Description of Artificial Sequence: Synthetic oligonucleotide”
uccacuuuuagaccuuaucc

SEQ ID NO: 428 - RNA, 20 nt, Artificial Sequence
  source: /note=“Description of Artificial Sequence: Synthetic oligonucleotide”
aauagcaucuacucuuguuc

SEQ ID NO: 429 - RNA, 20 nt, Artificial Sequence
  source: /note=“Description of Artificial Sequence: Synthetic oligonucleotide”
cucuuguucaggaaacaaug

SEQ ID NO: 430 - RNA, 20 nt, Artificial Sequence
  source: /note=“Description of Artificial Sequence: Synthetic oligonucleotide”
ggaaacaaugaggaccugac

SEQ ID NO: 431 - RNA, 20 nt, Artificial Sequence
  source: /note=“Description of Artificial Sequence: Synthetic oligonucleotide”
gaaacaaugaggaccugacu

SEQ ID NO: 432 - RNA, 20 nt, Artificial Sequence
  source: /note=“Description of Artificial Sequence: Synthetic oligonucleotide”
accugacugggcaguaagag

SEQ ID NO: 433 - RNA, 20 nt, Artificial Sequence
  source: /note=“Description of Artificial Sequence: Synthetic oligonucleotide”
accacucuuacugcccaguc

SEQ ID NO: 434 - RNA, 20 nt, Artificial Sequence
  source: /note=“Description of Artificial Sequence: Synthetic oligonucleotide”
aagaguggugauuaauagau

SEQ ID NO: 435 - RNA, 20 nt, Artificial Sequence
  source: /note=“Description of Artificial Sequence: Synthetic oligonucleotide”
agaguggugauuaauagaua

SEQ ID NO: 436 - RNA, 20 nt, Artificial Sequence
  source: /note=“Description of Artificial Sequence: Synthetic oligonucleotide”
agaaucgaacuguugauuag

SEQ ID NO: 437 - RNA, 20 nt, Artificial Sequence
  source: /note=“Description of Artificial Sequence: Synthetic oligonucleotide”
ucgaacuguugauuagaggu

SEQ ID NO: 438 - RNA, 20 nt, Artificial Sequence
  source: /note=“Description of Artificial Sequence: Synthetic oligonucleotide”
cgaacuguugauuagaggua

SEQ ID NO: 439 - RNA, 20 nt, Artificial Sequence
  source: /note=“Description of Artificial Sequence: Synthetic oligonucleotide”
augauuuuaaucugugaccu

SEQ ID NO: 440 - RNA, 20 nt, Artificial Sequence
  source: /note=“Description of Artificial Sequence: Synthetic oligonucleotide”
uaaucugugaccuuggugaa

SEQ ID NO: 441 - RNA, 20 nt, Artificial Sequence
  source: /note=“Description of Artificial Sequence: Synthetic oligonucleotide”
aaucugugaccuuggugaau

SEQ ID NO: 442 - RNA, 20 nt, Artificial Sequence
  source: /note=“Description of Artificial Sequence: Synthetic oligonucleotide”
agcuacuugcccauucacca

SEQ ID NO: 443 - RNA, 20 nt, Artificial Sequence
  source: /note=“Description of Artificial Sequence: Synthetic oligonucleotide”
uagcuaucuaaugacuaaaa

SEQ ID NO: 444 - RNA, 20 nt, Artificial Sequence
  source: /note=“Description of Artificial Sequence: Synthetic oligonucleotide”
augacuaaaauggaaaacac

SEQ ID NO: 445 - RNA, 20 nt, Artificial Sequence
  source: /note=“Description of Artificial Sequence: Synthetic oligonucleotide”
aaauacccaugcugagucug

SEQ ID NO: 446 - RNA, 20 nt, Artificial Sequence
  source: /note=“Description of Artificial Sequence: Synthetic oligonucleotide”
aggcaccucagacucagcau

SEQ ID NO: 447 - RNA, 20 nt, Artificial Sequence
  source: /note=“Description of Artificial Sequence: Synthetic oligonucleotide”
uaggcaccucagacucagca

SEQ ID NO: 448 - RNA, 20 nt, Artificial Sequence
  source: /note=“Description of Artificial Sequence: Synthetic oligonucleotide”
gcugagucugaggugccuau

SEQ ID NO: 449 - RNA, 20 nt, Artificial Sequence
  source: /note=“Description of Artificial Sequence: Synthetic oligonucleotide”
uauuuauauagauguccuau

SEQ ID NO: 450 - RNA, 20 nt, Artificial Sequence
  source: /note=“Description of Artificial Sequence: Synthetic oligonucleotide”
cauauaucaaacaauguacu

SEQ ID NO: 451 - RNA, 20 nt, Artificial Sequence
  source: /note=“Description of Artificial Sequence: Synthetic oligonucleotide”
ccaguacauuguuugauaua

SEQ ID NO: 452 - RNA, 20 nt, Artificial Sequence
  source: /note=“Description of Artificial Sequence: Synthetic oligonucleotide”
ccauauaucaaacaauguac

SEQ ID NO: 453 - RNA, 20 nt, Artificial Sequence
  source: /note=“Description of Artificial Sequence: Synthetic oligonucleotide”
caguacauuguuugauauau

SEQ ID NO: 454 - RNA, 20 nt, Artificial Sequence
  source: /note=“Description of Artificial Sequence: Synthetic oligonucleotide”
cauuguuugauauauggguu

SEQ ID NO: 455 - RNA, 20 nt, Artificial Sequence
  source: /note=“Description of Artificial Sequence: Synthetic oligonucleotide”
gauauauggguuuggcacug

SEQ ID NO: 456 - RNA, 20 nt, Artificial Sequence
  source: /note=“Description of Artificial Sequence: Synthetic oligonucleotide”
uauggguuuggcacugaggu

SEQ ID NO: 457 - RNA, 20 nt, Artificial Sequence
  source: /note=“Description of Artificial Sequence: Synthetic oligonucleotide”
ggguuuggcacugagguugg

SEQ ID NO: 458 - RNA, 20 nt, Artificial Sequence
  source: /note=“Description of Artificial Sequence: Synthetic oligonucleotide”
gcacugagguuggaggucag

SEQ ID NO: 459 - RNA, 20 nt, Artificial Sequence
  source: /note=“Description of Artificial Sequence: Synthetic oligonucleotide”
cagagguuagaaaucagagu

SEQ ID NO: 460 - RNA, 20 nt, Artificial Sequence
  source: /note=“Description of Artificial Sequence: Synthetic oligonucleotide”
agagguuagaaaucagaguu

SEQ ID NO: 461 - RNA, 20 nt, Artificial Sequence
  source: /note=“Description of Artificial Sequence: Synthetic oligonucleotide”
uagaaaucagaguugggaau

SEQ ID NO: 462 - RNA, 20 nt, Artificial Sequence
  source: /note=“Description of Artificial Sequence: Synthetic oligonucleotide”
agaaaucagaguugggaauu

SEQ ID NO: 463 - RNA, 20 nt, Artificial Sequence
  source: /note=“Description of Artificial Sequence: Synthetic oligonucleotide”
guugggaauugggauuauac

SEQ ID NO: 464 - RNA, 20 nt, Artificial Sequence
  source: /note=“Description of Artificial Sequence: Synthetic oligonucleotide”
cuuuguauucaucacacucu

SEQ ID NO: 465 - RNA, 20 nt, Artificial Sequence
  source: /note=“Description of Artificial Sequence: Synthetic oligonucleotide”
augaauacaaaguuaaauga

SEQ ID NO: 466 - RNA, 20 nt, Artificial Sequence
  source: /note=“Description of Artificial Sequence: Synthetic oligonucleotide”
uaaauguugguguucauuaa

SEQ ID NO: 467 - RNA, 20 nt, Artificial Sequence
  source: /note=“Description of Artificial Sequence: Synthetic oligonucleotide”
ugagauuucacauuaaaugu

SEQ ID NO: 468 - RNA, 20 nt, Artificial Sequence
  source: /note=“Description of Artificial Sequence: Synthetic oligonucleotide”
acauuuaaugugaaaucuca

SEQ ID NO: 469 - RNA, 20 nt, Artificial Sequence
  source: /note=“Description of Artificial Sequence: Synthetic oligonucleotide”
uaaaaucaucggggauuuug

SEQ ID NO: 470 - RNA, 20 nt, Artificial Sequence
  source: /note=“Description of Artificial Sequence: Synthetic oligonucleotide”
cuaaaaucaucggggauuuu

SEQ ID NO: 471 - RNA, 20 nt, Artificial Sequence
  source: /note=“Description of Artificial Sequence: Synthetic oligonucleotide”
ucuaaaaucaucggggauuu

SEQ ID NO: 472 - RNA, 20 nt, Artificial Sequence
  source: /note=“Description of Artificial Sequence: Synthetic oligonucleotide”
acugaguucuaaaaucaucg

SEQ ID NO: 473 - RNA, 20 nt, Artificial Sequence
  source: /note=“Description of Artificial Sequence: Synthetic oligonucleotide”
uacugaguucuaaaaucauc

SEQ ID NO: 474 - RNA, 20 nt, Artificial Sequence
  source: /note=“Description of Artificial Sequence: Synthetic oligonucleotide”
auacugaguucuaaaaucau

SEQ ID NO: 475 - RNA, 20 nt, Artificial Sequence
  source: /note=“Description of Artificial Sequence: Synthetic oligonucleotide”
uaauuaguguaaugccaaug

SEQ ID NO: 476 - RNA, 20 nt, Artificial Sequence
  source: /note=“Description of Artificial Sequence: Synthetic oligonucleotide”
aauuaguguaaugccaaugu

SEQ ID NO: 477 - RNA, 20 nt, Artificial Sequence
  source: /note=“Description of Artificial Sequence: Synthetic oligonucleotide”
aaugccaauguggguuagaa

SEQ ID NO: 478 - RNA, 20 nt, Artificial Sequence
  source: /note=“Description of Artificial Sequence: Synthetic oligonucleotide”
acuuccauucuaacccacau

SEQ ID NO: 479 - RNA, 20 nt, Artificial Sequence
  source: /note=“Description of Artificial Sequence: Synthetic oligonucleotide”
aauggaagucaacuugcugu

SEQ ID NO: 480 - RNA, 20 nt, Artificial Sequence
  source: /note=“Description of Artificial Sequence: Synthetic oligonucleotide”
cuugcuguugguuucagagc

SEQ ID NO: 481 - RNA, 20 nt, Artificial Sequence
  source: /note=“Description of Artificial Sequence: Synthetic oligonucleotide”
cuguugguuucagagcaggu

SEQ ID NO: 482 - RNA, 20 nt, Artificial Sequence
  source: /note=“Description of Artificial Sequence: Synthetic oligonucleotide”
uucagagcagguaggagaua

SEQ ID NO: 483 - RNA, 20 nt, Artificial Sequence
  source: /note=“Description of Artificial Sequence: Synthetic oligonucleotide”
agugaaaagcugaaacaaaa

SEQ ID NO: 484 - RNA, 20 nt, Artificial Sequence
  source: /note=“Description of Artificial Sequence: Synthetic oligonucleotide”
aagcugaaacaaaaaggaaa

SEQ ID NO: 485 - RNA, 20 nt, Artificial Sequence
  source: /note=“Description of Artificial Sequence: Synthetic oligonucleotide”
ugaaacaaaaaggaaaaggu

SEQ ID NO: 486 - RNA, 20 nt, Artificial Sequence
  source: /note=“Description of Artificial Sequence: Synthetic oligonucleotide”
gaaacaaaaaggaaaaggua

SEQ ID NO: 487 - RNA, 20 nt, Artificial Sequence
  source: /note=“Description of Artificial Sequence: Synthetic oligonucleotide”
ggaaaagguagggugaaaga

SEQ ID NO: 488 - RNA, 20 nt, Artificial Sequence
  source: /note=“Description of Artificial Sequence: Synthetic oligonucleotide”
gaaaagguagggugaaagau

SEQ ID NO: 489 - RNA, 20 nt, Artificial Sequence
  source: /note=“Description of Artificial Sequence: Synthetic oligonucleotide”
aaagaugggaaauguaugua

SEQ ID NO: 490 - RNA, 20 nt, Artificial Sequence
  source: /note=“Description of Artificial Sequence: Synthetic oligonucleotide”
gaugggaaauguauguaagg

SEQ ID NO: 491 - RNA, 20 nt, Artificial Sequence
  source: /note=“Description of Artificial Sequence: Synthetic oligonucleotide”
uguaaggaggaugagccaca

SEQ ID NO: 492 - RNA, 20 nt, Artificial Sequence
  source: /note=“Description of Artificial Sequence: Synthetic oligonucleotide”
ggaggaugagccacauggua

SEQ ID NO: 493 - RNA, 20 nt, Artificial Sequence
  source: /note=“Description of Artificial Sequence: Synthetic oligonucleotide”
gaggaugagccacaugguau

SEQ ID NO: 494 - RNA, 20 nt, Artificial Sequence
  source: /note=“Description of Artificial Sequence: Synthetic oligonucleotide”
gaugagccacaugguauggg

SEQ ID NO: 495 - RNA, 20 nt, Artificial Sequence
  source: /note=“Description of Artificial Sequence: Synthetic oligonucleotide”
aguauaccucccauaccaug

SEQ ID NO: 496 - RNA, 20 nt, Artificial Sequence
  source: /note=“Description of Artificial Sequence: Synthetic oligonucleotide”
augguaugggagguauacua

SEQ ID NO: 497 - RNA, 20 nt, Artificial Sequence
  source: /note=“Description of Artificial Sequence: Synthetic oligonucleotide”
ggagguauacuaaggacucu

SEQ ID NO: 498 - RNA, 20 nt, Artificial Sequence
  source: /note=“Description of Artificial Sequence: Synthetic oligonucleotide”
gagguauacuaaggacucua

SEQ ID NO: 499 - RNA, 20 nt, Artificial Sequence
  source: /note=“Description of Artificial Sequence: Synthetic oligonucleotide”
acucuagggucagagaaaua

SEQ ID NO: 500 - RNA, 20 nt, Artificial Sequence
  source: /note=“Description of Artificial Sequence: Synthetic oligonucleotide”
cucuagggucagagaaauau

SEQ ID NO: 501 - RNA, 20 nt, Artificial Sequence
  source: /note=“Description of Artificial Sequence: Synthetic oligonucleotide”
aagaaugugaauuuuguaga

SEQ ID NO: 502 - RNA, 20 nt, Artificial Sequence
  source: /note=“Description of Artificial Sequence: Synthetic oligonucleotide”
uucuacaaaauucacauucu

SEQ ID NO: 503 - RNA, 20 nt, Artificial Sequence
  source: /note=“Description of Artificial Sequence: Synthetic oligonucleotide”
acaaaauucacauucuuggc

SEQ ID NO: 504 - RNA, 20 nt, Artificial Sequence
  source: /note=“Description of Artificial Sequence: Synthetic oligonucleotide”
caaaauucacauucuuggcu

SEQ ID NO: 505 - RNA, 20 nt, Artificial Sequence
  source: /note=“Description of Artificial Sequence: Synthetic oligonucleotide”
uucacauucuuggcugggug

SEQ ID NO: 506 - RNA, 20 nt, Artificial Sequence
  source: /note=“Description of Artificial Sequence: Synthetic oligonucleotide”
aggguggaucaccugauguu

SEQ ID NO: 507 - RNA, 20 nt, Artificial Sequence
  source: /note=“Description of Artificial Sequence: Synthetic oligonucleotide”
gaucucgaacuccuaacauc

SEQ ID NO: 508 - DNA, 5000 bp, Artificial Sequence
  source: /note=“Description of Artificial Sequence: Synthetic polynucleotide”
  misc_feature (1)..(5000): /note=“This sequence may encompass 50-5000 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 509 - protein, 7 aa, Simian virus 40
PKKKRKV

SEQ ID NO: 510 - protein, 6 aa, Artificial Sequence
  source: /note=“Description of Artificial Sequence: Synthetic 6xHis tag”
HHHHHH

SEQ ID NO: 511 - protein, 8 aa, Artificial Sequence
  source: /note=“Description of Artificial Sequence: Synthetic 8xHis tag”
HHHHHHHH

SEQ ID NO: 512 - RNA, 42 nt, Artificial Sequence
  source: /note=“Description of Artificial Sequence: Synthetic oligonucleotide”
  modified_base (1)..(20): a, c, u, g, unknown or other
nnnnnnnnnnnnnnnnnnnnguuuuagagcuaugcuguuuug

SEQ ID NO: 513 - RNA, 86 nt, Artificial Sequence
  source: /note=“Description of Artificial Sequence: Synthetic oligonucleotide”
  misc_feature (80)..(86): /note=“This region may encompass 1-7 nucleotides”
aacuuaccaaggaacagcauagcaaguuaaaauaaggcuaguccguuaucaacuugaaaaaguggcaccgagucggugcuuuuuuu

SEQ ID NO: 514 - RNA, 74 nt, Artificial Sequence
  source: /note=“Description of Artificial Sequence: Synthetic oligonucleotide”
  misc_feature (68)..(74): /note=“This region may encompass 1-7 nucleotides”
aacagcauagcaaguuaaaauaaggcuaguccguuaucaacuugaaaaaguggcaccgagucggugcuuuuuuu

SEQ ID NO: 515 - RNA, 102 nt, Artificial Sequence
  source: /note=“Description of Artificial Sequence: Synthetic polynucleotide”
  modified_base (1)..(19): a, c, u, g, unknown or other
  misc_feature (96)..(102): /note=“This region may encompass 1-7 nucleotides”
nnnnnnnnnnnnnnnnnnnguuuuagagcuagaaauagcaaguuaaaauaaggcuaguccguuaucaacuugaaaaaguggcaccgagucggugcuuuuuuu

SEQ ID NO: 516 - DNA, 21 bp, Artificial Sequence
  source: /note=“Description of Artificial Sequence: Synthetic oligonucleotide”
  modified_base (1)..(14): a, c, t or g
  modified_base (15)..(16): a, c, t, g, unknown or other
nnnnnnnnnnnnnnnnagaaw

SEQ ID NO: 517 - DNA, 13 bp, Artificial Sequence
  source: /note=“Description of Artificial Sequence: Synthetic oligonucleotide”
  modified_base (1)..(6): a, c, t or g
  modified_base (7)..(8): a, c, t, g, unknown or other
nnnnnnnnagaaw

SEQ ID NO: 518 - DNA, 21 bp, Artificial Sequence
  source: /note=“Description of Artificial Sequence: Synthetic oligonucleotide”
  modified_base (1)..(14): a, c, t or g
  modified_base (15)..(16): a, c, t, g, unknown or other
nnnnnnnnnnnnnnnnagaaw

SEQ ID NO: 519 - DNA, 18 bp, Artificial Sequence
  source: /note=“Description of Artificial Sequence: Synthetic oligonucleotide”
  modified_base (1)..(11): a, c, t or g
  modified_base (12)..(13): a, c, t, g, unknown or other
nnnnnnnnnnnnnagaaw

SEQ ID NO: 520 - RNA, 19 nt, Artificial Sequence
  source: /note=“Description of Artificial Sequence: Synthetic oligonucleotide”
uuugccuugucaaggcuau

SEQ ID NO: 521 - RNA, 19 nt, Artificial Sequence
  source: /note=“Description of Artificial Sequence: Synthetic oligonucleotide”
aaggcuauuggucaaggca

SEQ ID NO: 522 - RNA, 19 nt, Artificial Sequence
  source: /note=“Description of Artificial Sequence: Synthetic oligonucleotide”
cuauuggucaaggcaaggc

SEQ ID NO: 523 - protein, 9 aa, Unknown
  source: /note=“Description of Unknown: ′LAGLIDADG′ family motif”
LAGLIDADG

SEQ ID NO: 524 - DNA, 20 bp, Homo sapiens
cacgacccaaccctaatcag

SEQ ID NO: 525 - DNA, 20 bp, Homo sapiens
cacgcccacaccttaatcag

SEQ ID NO: 526 - DNA, 20 bp, Homo sapiens
tttggcctaggattagggtg

SEQ ID NO: 527 - DNA, 20 bp, Homo sapiens
tttggcctgtgagtagggtg

SEQ ID NO: 528 - DNA, 20 bp, Homo sapiens
tgtggcctctgattaggatg

SEQ ID NO: 529 - DNA, 20 bp, Homo sapiens
ttttgtcacaggctccagta

SEQ ID NO: 530 - DNA, 20 bp, Homo sapiens
tttatcacatgctccaggac

SEQ ID NO: 531 - DNA, 20 bp, Homo sapiens
tttataacaggctccagaaa

SEQ ID NO: 532 - DNA, 20 bp, Homo sapiens
cacaggctcctggaaggctt

SEQ ID NO: 533 - DNA, 20 bp, Homo sapiens
tggggtggggagatatgaag

SEQ ID NO: 534 - DNA, 20 bp, Homo sapiens
gcaagcatttaagtggcaac

SEQ ID NO: 535 - DNA, 20 bp, Homo sapiens
gaaacaatgaggacctgtgt

SEQ ID NO: 536 - DNA, 20 bp, Homo sapiens
gaaacactgagcacctgact

SEQ ID NO: 537 - DNA, 20 bp, Homo sapiens
aggcacctcagactgagcac

SEQ ID NO: 538 - DNA, 20 bp, Homo sapiens
aggcacctcagattcagcac

SEQ ID NO: 539 - DNA, 20 bp, Homo sapiens
aggcacctcacactcaacat

SEQ ID NO: 540 - DNA, 20 bp, Homo sapiens
aggtacctcaaactcagcat

SEQ ID NO: 541 - DNA, 20 bp, Homo sapiens
agtcctggtatcttctatgg

SEQ ID NO: 542 - DNA, 20 bp, Homo sapiens
agccctggtttcctctatga

SEQ ID NO: 543 - DNA, 20 bp, Homo sapiens
agtcctggaatcctatatga

SEQ ID NO: 544 - DNA, 20 bp, Homo sapiens
ggagaagaaaactagctaaa

SEQ ID NO: 545 - DNA, 20 bp, Homo sapiens
gggagaagaaaactagctaa

SEQ ID NO: 546 - DNA, 20 bp, Homo sapiens
ggagaagaaaactagttaga

SEQ ID NO: 547 - DNA, 20 bp, Homo sapiens
ggtgaaaaaaactagctaaa

SEQ ID NO: 548 - DNA, 20 bp, Homo sapiens
ggagaagaaaaatagctgaa

SEQ ID NO: 549 - DNA, 20 bp, Homo sapiens
ggagaaggaaactagctaaa

SEQ ID NO: 550 - DNA, 22 bp, Homo sapiens
tttgccttgtcaaggctattgg

SEQ ID NO: 551 - DNA, 23 bp, Homo sapiens
cttgtcaaggctattggtcaagg

SEQ ID NO: 552 - DNA, 23 bp, Homo sapiens
cttgaccaatagccttgacaagg

SEQ ID NO: 553 - DNA, 22 bp, Homo sapiens
aaggctattggtcaaggcaagg

SEQ ID NO: 554 - DNA, 22 bp, Homo sapiens
ctattggtcaaggcaaggctgg

SEQ ID NO: 555 - DNA, 23 bp, Homo sapiens
ctaacagttgcttttatcacagg

SEQ ID NO: 556 - DNA, 23 bp, Homo sapiens
actgaatcggaacaaggcaaagg

SEQ ID NO: 557 - DNA, 23 bp, Homo sapiens
atcagaggccaaacccttcctgg

(19) United States
(12) Patent Application Publication
(10) Pub. No.: US 2020/0140896 A1
(43) Pub. Date: May 7, 2020

(54) METHODS FOR THE TREATMENT OF DISEASE WITH GENE EDITING SYSTEMS

(71) Applicants: Novartis AG, Basel (CH); Intellia Therapeutics, Inc., Cambridge, MA (US)

(72) Inventors: Nicole RENAUD, Somerville, MA (US); Xiaojun ZHAO, Quincy, MA (US)

(21) Appl. No.: 16/625,098

(22) PCT Filed: Jun. 28, 2018
(86) PCT No.: PCT/IB2018/054831

§ 371 Date: Dec. 20, 2019

Related U.S. Application Data
(60) Provisional application No. 62/527,978, filed on Jun. 30, 2017.

(51) Int. Cl. C12N 15/90; C12N 15/10; C12N 15/11; A61K 35/761; A61K 35/17; A61P 7/00

(52) U.S. Cl. CPC C12N 15/907; C12N 15/102; A61P 7/00; A61K 35/761; A61K 35/17; C12N 15/111

(57) ABSTRACT

Provided herein are methods of selectively treating a patient with a gene editing system on the basis of ascertaining the presence of a target sequence, at a target locus, that is fully complementary to a targeting domain of said gene editing system and/or on the basis of ascertaining the absence of a target sequence, at a locus other than the target locus, that is fully complementary to a targeting domain of said gene editing system.

### RELATED APPLICATIONS

This application claims priority to U.S. Provisional patent application No. 62/527,978, filed Jun. 30, 2017, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Jun. 22, 2018, is named PAT057802-WO-PCT_SL.txt and is 139,886 bytes in size.

### BACKGROUND

CRISPRs (Clustered Regularly Interspaced Short Palindromic Repeats) evolved in bacteria as an adaptive immune system to defend against viral attack. Upon exposure to a virus, short segments of viral DNA are integrated into the CRISPR locus of the bacterial genome. RNA is transcribed from a portion of the CRISPR locus that includes the viral sequence. That RNA, which contains sequence complimentary to the viral genome, mediates targeting of a Cas9 protein to the sequence in the viral genome. The Cas9 protein cleaves and thereby silences the viral target.

Recently, the CRISPR/Cas system has been adapted for genome editing in eukaryotic cells. The introduction of site-specific single (SSBs) or double strand breaks (DSBs) allows for target sequence alteration through, for example, non-homologous end-joining (NHEJ) or homology-directed repair (HDR).

### SUMMARY OF THE INVENTION

Without being bound by theory the invention is based in part of the finding that the editing efficiency of a gene editing system may be drastically reduced when even a single mismatch nucleotide is present in the target sequence of the gene editing system, which can drastically reduce efficacy of the system. As well, the invention is based at least in part on the recognition that variant sequences may be present in the genomes of individuals who may be candidates for a therapy comprising genome editing, and that response to that therapy may be in part dependent upon the absence of a variant sequence at a target sequence of a gene editing system. In addition, it may be beneficial to target regions where polymorphisms may exist. Thus, without being bound by theory, it is recognized herein that it is beneficial to selectively treat patients with gene editing systems based on the presence of a fully complementary target sequence at the target locus, preferably within the cells of interest. The invention thus provides for such improved methods of treatment with gene editing systems.

In an aspect, the invention provides a method of selectively treating a patient with a gene editing system, including:
a) selectively introducing said gene editing system into a cell, e.g., population of cells, of the patient on the basis of the cell, e.g., population of cells, including a target sequence, at a target locus, that is fully complementary to a targeting domain of said gene editing system; and/or
b) selectively introducing said gene editing system to a cell, e.g., population of cells, of the patient on the basis of the cell, e.g., population of cells, not including a target sequence, at a locus other than the target locus, that is fully complementary to a targeting domain of said gene editing system.

In an aspect, the invention provides a method of selectively treating a patient with a gene editing system, including:
a) selecting the patient for treatment on the basis of one or more cells of the patient including a target sequence, at a target locus, that is fully complementary to a targeting domain of said gene editing system; and
b) thereafter, administering a therapeutically effective amount of said gene editing system to the patient or to a population of cells of said patient,

thereby inducing a modification at or near the target sequence at the target locus in a cell or the patient or a cell of the population of cells.

In an aspect, the invention provides a method of selectively treating a patient with a gene editing system including:
a) assaying one or more cells from a biological sample from the patient for the presence of a target sequence, at a target locus, that is fully complementary to a targeting domain of said gene editing system; and
b) thereafter, selectively administering a therapeutically effective amount of the gene editing system to the patient or to a cell of the patient:
   i) on the basis of one or more cells of the biological sample of the patient including a target sequence, at a target locus, that is fully complementary to a targeting domain of said gene editing system; and/or
   ii) on the basis of one or more cells of the biological sample from the patient not including a target sequence, at a locus other than the target locus, that is fully complementary to a targeting domain of said gene editing system,

thereby inducing a modification at or near the target sequence at the target locus in a cell or the patient or a cell of the population of cells.

In an aspect, the invention provides a method of selectively treating a patient with a gene editing system, including:
a) assaying one or more cells of a biological sample from the patient for at least one target sequence, at a target locus, that is fully complementary to the targeting domain of said gene editing system;
b) thereafter, selecting the patient for treatment with the gene editing system on the basis of one or more cells of the biological sample from the patient having the target sequence, at the target locus, that is fully complementary to the targeting domain of said gene editing system; and
c) thereafter, administering a therapeutically effective amount of the gene editing system of cells to the patient.

In an aspect, the invention provides a method according to any of the previous aspects, wherein the biological sample is selected from the group consisting of synovial fluid, blood, bone marrow, serum, feces, plasma, urine, tear, saliva, cerebrospinal fluid, an apheresis sample, a leukopheresis sample, a leukocyte sample and a tissue sample, for example is blood, an apheresis sample, a leukopheresis sample, a leukocyte sample, or bone marrow.

In an aspect, including in any of the previous aspects and embodiments, the step of assaying includes a technique selected from the group consisting of Next generation sequencing (NGS), pyrosequencing, Sanger sequencing, Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southern Blot, immunoassays, immunohistochemistry, ELISA, flow cytometry, Western blot, HPLC, and mass spectrometry.

In embodiments, the gene editing system is a zinc finger nuclease (ZFN) system, a TALEN system, a meganuclease system, or CRISPR system, for example (in each case), as described herein. CRISPR systems are particularly preferred.

In embodiments, including in any of the previous aspects and embodiments, the one or more cells include, e.g., consist of, hematopoietic stem and progenitor cells (HSPCs) or HSCs. In embodiments, including in any of the previous aspects and embodiments, the patient has a hemoglobinopathy, for example, sickle cell disease, sickle cell anemia, beta-thalassemia, thalassemia major, thalassemia intermedia. In embodiments, the target locus is the human globin locus, for example, the HBG1 promoter (Chr11:5,249,833-5,250,237 according to hg38) and/or HBG2 promoter (Chr11:5,254,738-5,255,164 according to hg38), or, for example, an HPFH region, or, for example, an AAVS1 locus, a BCL11a gene, or a BCL11a enhancer region (for example, a +55 region of the BCL11a enhancer (Chr2:60497676-60498941 according to hg38), a +58 region of the BCL11a enhancer (Chr2:60494251-60495546 according to hg38), or a +62 region of the BCL11a enhancer (Chr2:60490409-60491734 according to hg38)). In embodiments where the gene editing system is a CRISPR system, the CRISPR system includes a gRNA molecule including a targeting domain complementary to any one of SEQ ID NO: 1 to 161,197 of PCT Publication WO2017/077394. In exemplary embodiments where the gene editing system is a CRISPR system, the CRISPR system includes a gRNA molecule including a targeting domain complementary to any one of SEQ ID NO: 1 to 135 of PCT Publication WO2016/182917. In exemplary embodiments where the gene editing system is a CRISPR system, the CRISPR system includes a gRNA including a targeting domain sequence selected from the targeting domain sequences of Tables 1-3. In exemplary embodiments where the gene editing system is a ZFN system, the ZFN system includes a targeting domain complementary to any one of SEQ ID NO: 63-80 and 232-251 of PCT Publication WO2015/073683. In exemplary embodiments, where the gene editing system is a TALEN system, the TALEN system includes a targeting domain complementary to any one of SEQ ID NO: 7-11, 16-62, and 143-184 of PCT Publication WO2015/073683. In preferred embodiments, the target sequence is a target sequence identified in Table 6, and further preferably, the gene editing system is a CRISPR gene editing system (e.g., as described herein) comprising a gRNA molecule (e.g., as described herein) comprising a targeting domain listed in Table 6.

In other embodiments, including in any of the aforementioned aspects and embodiments, the patient has a cancer or autoimmune disease, for example, has cancer. In embodiments, the cell to be edited with the genome editing system is a cancer cell. In other embodiments, the cell to be edited is an immune effector cell, for example, a T cell or NK cell, for example a T cell. In embodiments, the cell has been, will be, or is further engineered to express a chimeric antigen receptor (CAR). In exemplary embodiments, the target locus (e.g., the target locus in a T cell), is selected from the group consisting of: TRAC, TRBC1, TRBC2, CD3E, CD3G, CD3D, B2M, CIITA, CD247, HLA-A, HLA-B, HLA-C, DCK, CD52, FKBP1A, NLRC5, RFXANK, RFX5, RFXAP, NR3C1, CD274, HAVCR2, LAG3, PDCD1, PD-L2, CTLA4, CEACAM (e.g., CEACAM-1, CEACAM-3 and/or CEACAM-5), VISTA, BTLA, TIGIT, LAIR1, CD160, 2B4, CD80, CD86, B7-H3 (CD113), B7-H4 (VTCN1), HVEM (TNFRSF14 or CD107), KIR, A2aR, MHC class I, MHC class II, GAL9, adenosine, and TGF beta, PTPN11, and combinations thereof. In exemplary embodiments where the gene editing system is a CRISPR system, the CRISPR system includes a gRNA molecule including a targeting domain described in PCT Publication WO/2017/093969, for example, described in any of Tables 1-6 and 6b-g of WO2017/093969.

In an aspect, the invention provides gene editing system for use in treating a patient having a disease, characterized in that a therapeutically effective amount of the gene editing system is to be administered to the patient (or cells of the patient) on the basis of a cell of said patient including a target sequence, at a target locus, that is fully complementary to a targeting domain of said gene editing system.

In an aspect, the invention provides a gene editing system for use in treating a patient having a disease, characterized in that:
a) the patient is to be selected for treatment with the gene editing system on the basis of a cell of said patient including a target sequence, at a target locus, that is fully complementary to a targeting domain of said gene editing system; and
b) thereafter, a therapeutically effective amount of the gene editing system is to be administered to the patient.

In an aspect, the invention provides a gene editing system for use in treating a patient having a disease, characterized in that:
a) a cell of a biological sample from the patient is to be assayed for at least one a target sequence, at a target locus, that is fully complementary to a targeting domain of said gene editing system; and
b) a therapeutically effective amount of the gene editing system is to be selectively administered to the patient on the basis of the cell of the biological sample from the patient having the at least one a target sequence, at a target locus, that is fully complementary to a targeting domain of said gene editing system.

In an aspect, the invention provides a gene editing system for use in treating a patient having a disease, characterized in that:
a) a cell of a biological sample from the patient is to be assayed for at least one a target sequence, at a target locus, that is fully complementary to a targeting domain of said gene editing system;
b) the patient is selected for treatment with the gene editing system on the basis of the cell of the biological sample from the patient having the at least one a target sequence, at a target locus, that is fully complementary to a targeting domain of said gene editing system; and
c) a therapeutically effective amount of the gene editing system is to be selectively administered to the patient.

In an aspect, the invention provides a method of predicting the likelihood that a patient having an disease will respond to treatment with a gene editing system, including assaying a cell of a biological sample from the patient for the presence or absence of at least one target sequence, at a target locus, that is fully complementary to a targeting domain of said gene editing system, wherein:
a) the presence of the at least one target sequence, at a target locus, that is fully complementary to a targeting domain of said gene editing system is indicative of an increased likelihood that the patient will respond to treatment with the gene editing system; and
b) the absence of the at least one target sequence, at a target locus, that is fully complementary to a targeting domain of said gene editing system is indicative of a decreased likelihood that the patient will respond to treatment with the gene editing system.

In embodiments, the above methods further include the step of obtaining the biological sample from the patient, wherein the step of obtaining is performed prior to the step of assaying, for example, assaying from a biological sample selected from the group consisting of synovial fluid, blood, bone marrow, serum, feces, plasma, urine, tear, saliva, cerebrospinal fluid, an apheresis sample, a leukopheresis sample, a leukocyte sample and a tissue sample, for example, blood, an apheresis sample, a leukopheresis sample, a leukocyte sample, or bone marrow.

In embodiments, including an any of the aforementioned aspects and embodiments, the step of assaying includes a technique selected from the group consisting of Next generation sequencing (NGS), pyrosequencing, Sanger sequencing, Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southern Blot, immunoassays, immunohistochemistry, ELISA, flow cytometry, Western blot, HPLC, and mass spectrometry.

### DETAILED DESCRIPTION

As used herein, the term “gene editing system” or “genome editing system” refers to a system comprising one or more DNA-binding domains or components and one or more DNA-modifying domains or components, or isolated nucleic acids, e.g., one or more vectors, encoding said DNA-binding and DNA-modifying domains or components. Gene editing systems are used for modifying the nucleic acid of a target gene and/or for modulating the expression of a target gene. In gene editing systems, for example, the one or more DNA-binding domains or components are associated with the one or more DNA-modifying domains or components, such that the one or more DNA-binding domains target the one or more DNA-modifying domains or components to a specific nucleic acid site. Gene editing systems include but are not limited to, zinc finger nucleases (ZFN) systems, transcription activator-like effector nucleases (TALENs); clustered regularly interspaced short palindromic repeats (CRISPR)/Cas systems, and meganuclease systems.

A “target sequence” of a gene editing system is a nucleic acid sequence that is complementary, e.g., fully complementary, to the targeting domain of a gene editing system. In the case of a CRISPR system, in some embodiments, the target sequence is a sequence that is complementary, e.g., fully complementary, to the gRNA targeting domain sequence. In other embodiments, in the case of a CRISPR system, the target sequence is a sequence that is complementary, e.g., fully complementary, to the gRNA targeting domain sequence together with the protospacer adjacent motif (PAM) sequence recognized by the Cas molecule of the CRISPR system. In the case of a ZFN system, TALEN system, or meganuclease system, the target sequence is a sequence that matches the sequence intended to be recognized by the system (and, as with a CRISPR system), may include the sequence recognized by the nuclease domain of the system.

The term “targeting domain,” when used in connection with a gene editing system, refers to the portion of the gene editing system which recognizes, e.g., binds to, a target nucleic acid in a sequence-dependent manner. Each gene editing system is designed to bind to a specific fully complementary target sequence. As the term is used in connection with a gRNA, the targeting domain is the portion of the gRNA molecule that recognizes, e.g., is complementary to, a target sequence, e.g., a target sequence within the nucleic acid of a cell, e.g., within a gene.

The term “complementary” as used in connection with nucleic acid, refers to the pairing of bases, A with T or U, and G with C. The term complementary refers to nucleic acid molecules that are completely complementary (“fully complementary”), that is, form A to T or U pairs and G to C pairs across the entire reference sequence, as well as molecules that are at least 80%, 85%, 90%, 95%, 99% complementary. With reference to protein recognition of nucleic acid (for example, in the case of a ZFN system, TALEN system, or meganuclease system), the term complementary refers to the degree to which the nucleic acid sequence matches the intended target sequence of the protein. Thus, in this context, “fully complementary” means that the sequence of nucleic acid matches the intended target sequence across its full length.

The term “target locus” refers to the site to which a gene editing system is intended to bind. In embodiments, the target locus is a gene. In such embodiments, a target locus may be defined by the gene name or the name of the protein encoded by said gene (for example, with reference to a UniProt, OMIM, Ensembl, Entrez Gene or HGNC identifier), or by the specific genomic coordinates encompassing the locus. In other embodiments, the target locus is a regulatory region such as a promoter or a tissue-specific enhancer or repressor of transcription. In other embodiments, the target locus is a specific region of intergenic DNA. A target locus may be identified by a range of genomic coordinates encompassing the locus, for example, with reference to a reference genome, for example, hg38.

A “modification” as the term is used in connection with a nucleic acid, e.g., a target sequence, refers to a chemical difference at or near the target sequence relative to its natural state. In embodiments, a modification comprises an indel. In embodiments, a modification comprises a DNA strand break.

An “indel,” as the term is used herein, refers to a nucleic acid comprising one or more insertions of nucleotides, one or more deletions of nucleotides, or a combination of insertions and deletions of nucleotides, relative to a reference nucleic acid, that results after being exposed to a gene editing system, for example a CRISPR system. Indels can be determined by sequencing nucleic acid after being exposed to a gene editing system, for example, by NGS. With respect to the site of an indel, an indel is said to be “at or near” a sequence if it comprises at least one insertion or deletion within about 10, 9, 8, 7, 6, 5, 4, 3, 2, or 1 nucleotide(s) of the reference site (e.g., the target sequence), or is overlapping with part or all of said reference site (e.g., target sequence) (e.g., comprises at least one insertion or deletion overlapping with, or within 10, 9, 8, 7, 6, 5, 4, 3, 2, or 1 nucleotides of a site complementary to the targeting domain of a gene editing system, e.g., a CRISPR system, e.g., described herein).

An “indel pattern,” as the term is used herein, refers to a set of indels that results after exposure to a gene editing system. In an embodiment, the indel pattern comprises, e.g., consists of, the top three indels, by frequency of appearance in a population of cells. In an embodiment, the indel pattern comprises, e.g., consists of, the top five indels, by frequency of appearance in a population of cells. In an embodiment, the indel pattern comprises, e.g., consists of, the indels which are present at greater than about 5% frequency relative to all sequencing reads. In an embodiment, the indel pattern comprises, e.g., consists of, the indels which are present at greater than about 10% frequency relative to total number of indel sequencing reads (i.e., those reads that do not consist of the unmodified reference nucleic acid sequence). In an embodiment, the indel pattern comprises, e.g., consists of, any 3 of the top five most frequently observed indels. The indel pattern may be determined, for example, by sequencing cells of a population of cells which were exposed to the gRNA molecule.

An “off-target indel,” as the term is used herein, refers to an indel at or near a site other than the target sequence of the targeting domain of the gene editing system. Such sites may comprise, for example, 1, 2, 3, 4, 5 or more mismatch nucleotides relative to the sequence of the targeting domain of the gRNA. In exemplary embodiments, such sites are detected using targeted sequencing of in silico predicted off-target sites, or by an insertional method known in the art.

The terms “CRISPR system,” “Cas system” or “CRISPR/Cas system” refer to a set of molecules comprising an RNA-guided nuclease or other effector molecule and a gRNA molecule that together are necessary and sufficient to direct and effect modification of nucleic acid at a target sequence by the RNA-guided nuclease or other effector molecule. In one embodiment, a CRISPR system comprises a gRNA and a Cas protein, e.g., a Cas9 protein. Such systems comprising a Cas9 or modified Cas9 molecule are referred to herein as “Cas9 systems” or “CRISPR/Cas9 systems.” In one example, the gRNA molecule and Cas molecule may be complexed, to form a ribonuclear protein (RNP) complex.

The terms “guide RNA,” “guide RNA molecule,” “gRNA molecule” or “gRNA” are used interchangeably, and refer to a set of nucleic acid molecules that promote the specific directing of a RNA-guided nuclease or other effector molecule (typically in complex with the gRNA molecule) to a target sequence. In some embodiments, said directing is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In embodiments, a gRNA molecule consists of a single contiguous polynucleotide molecule, referred to herein as a “single guide RNA” or “sgRNA” and the like. In other embodiments, a gRNA molecule consists of a plurality, usually two, polynucleotide molecules, which are themselves capable of association, usually through hybridization, referred to herein as a “dual guide RNA” or “dgRNA,” and the like. gRNA molecules are described in more detail below, but generally include a targeting domain and a tracr. In embodiments the targeting domain and tracr are disposed on a single polynucleotide. In other embodiments, the targeting domain and tracr are disposed on separate polynucleotides.

The term “targeting domain” as the term is used in connection with a gRNA, is the portion of the gRNA molecule that recognizes, e.g., is complementary to, a target sequence, e.g., a target sequence within the nucleic acid of a cell, e.g., within a gene.

The term “crRNA” as the term is used in connection with a gRNA molecule, is a portion of the gRNA molecule that comprises a targeting domain and a region that interacts with a tracr to form a flagpole region.

The term “flagpole” as used herein in connection with a gRNA molecule, refers to the portion of the gRNA where the crRNA and the tracr bind to, or hybridize to, one another.

The term “tracr” as used herein in connection with a gRNA molecule, refers to the portion of the gRNA that binds to a nuclease or other effector molecule. In embodiments, the tracr comprises nucleic acid sequence that binds specifically to Cas9. In embodiments, the tracr comprises nucleic acid sequence that forms part of the flagpole.

“Template Nucleic Acid” as used in connection with homology-directed repair or homologous recombination, refers to nucleic acid to be inserted at the site of modification by the CRISPR system donor sequence for gene repair (insertion) at site of cutting.

The term “BCL11a” refers to B-cell lymphoma/leukemia 11A, a RNA polymerase II core promoter proximal region sequence-specific DNA binding protein, and the gene encoding said protein, together with all introns and exons. This gene encodes a C2H2 type zinc-finger protein. BCL11A has been found to play a role in the suppression of fetal hemoglobin production. BCL11a is also known as B-Cell CLL/Lymphoma 11A (Zinc Finger Protein), CTIP1, EVI9, Ecotropic Viral Integration Site 9 Protein Homolog, COUP-TF-Interacting Protein 1, Zinc Finger Protein 856, KIAA1809, BCL-11A, ZNF856, EVI-9, and B-Cell CLL/Lymphoma 11A. The term encompasses all isoforms and splice variants of BLC11a. The human gene encoding BCL11a is mapped to chromosomal location 2p16.1 (by Ensembl). The human and murine amino acid and nucleic acid sequences can be found in a public database, such as GenBank, UniProt and Swiss-Prot., and the genomic sequence of human BCL11a can be found in GenBank at NC_000002.12. The BCL11a gene refers to this genomic location, including all introns and exons. There are multiple known isotypes of BCL11a.

The sequence of mRNA encoding isoform 1 of human BCL11a can be found at NM_022893. The peptide sequence of isoform 1 of human BCL11a is:

```
      10 20 30 40 50
MSRRKQGKPQ HLSKREFSPE PLEAILTDDE PDHGPLGAPE GDHDLLTCGQ
      60 70 80 90 100
CQMNFPLGDI LIFIEHKRKQ CNGSLCLEKA VDKPPSPSPI EMKKASNPVE
      110 120 130 140 150
VGIQVTPEDD DCLSTSSRGI CPKQEHIADK LLHWRGLSSP RSAHGALIPT
      160 170 180 190 200
PGMSAEYAPQ GICKDEPSSY TCTTCKQPFT SAWFLLQHAQ NTHGLRIYLE
    210 220 230 240 250
SEHGSPLTPR VGIPSGLGAE CPSQPPLHGI HIADNNPFNL LRIPGSVSRE
      260 270 280 290 300
ASGLAEGRFP PTPPLFSPPP RHHLDPHRIE RLGAEEMALA THHPSAFDRV
      310 320 330 340 350
LRLNPMAMEP PAMDFSRRLR ELAGNTSSPP LSPGRPSPMQ RLLQPFQPGS
      360 370 380 390 400
KPPFLATPPL PPLQSAPPPS QPPVKSKSCE FCGKTFKFQS NLVVHRRSHT
      410 420 430 440 450
GEKPYKCNLC DHACTQASKL KRHMKTHMHK SSPMTVKSDD GLSTASSPEP
      460 470 480 490 500
GTSDLVGSAS SALKSVVAKF KSENDPNLIP ENGDEEEEED DEEEEEEEEE
    510 520 530 540 550
EEEELTESER VDYGFGLSLE AARHHENSSR GAVVGVGDES RALPDVMQGM
      560 570 580 590 600
VLSSMQHFSE AFHQVLGEKH KRGHLAEAEG HRDTCDEDSV AGESDRIDDG
      610 620 630 640 650
TVNGRGCSPG ESASGGLSKK LLLGSPSSLS PFSKRIKLEK EFDLPPAAMP
    660 670 680 690 700
NTENVYSQWL AGYAASRQLK DPFLSFGDSR QSPFASSSEH SSENGSLRFS
      710 720 730 740 750
TPPGELDGGI SGRSGTGSGG STPHISGPGP GRPSSKEGRR SDTCEYCGKV
      760 770 780 790 800
FKNCSNLTVH RRSHTGERPY KCELCNYACA QSSKLTRHMK THGQVGKDVY
      810 820 830
KCEICKMPFS VYSTLEKHMK KWHSDRVLNN DIKTE
SEQ ID NO: 73 (Identifier Q9H165-1; and NM_022893.3; and accession ADL14508.1).
```

The sequences of other BCL11a protein isoforms are provided at:

Isoform 2: Q9H165-2

Isoform 3: Q9H165-3

Isoform 4: Q9H165-4

Isoform 5: Q9H165-5

Isoform 6: Q9H165-6

As used herein, a human BCL11a protein also encompasses proteins that have over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with BCL11a isoform 1-6, wherein such proteins still have at least one of the functions of BCL11a.

The term “globin locus” as used herein refers to the region of human chromosome 11 comprising genes for embryonic (ε), fetal (G(γ) and A(γ)), adult globin genes (γ and β), locus control regions and DNase I hypersensitivity sites.

The term “HPFH” refers to hereditary persistence of fetal hemoglobin, and is characterized in increased fetal hemoglobin in adult red blood cells. The term “HPFH region” refers to a genomic site which, when modified (e.g., mutated or deleted), causes increased HbF production in adult red blood cells, and includes HPFH sites identified in the literature (see e.g., the Online Mendelian Inheritance in Man: http://www.omim.org/entry/141749). In an exemplary embodiment, the HPFH region is a region within or encompassing the beta globin gene cluster on chromosome 11p15. In an exemplary embodiment, the HPFH region is within or encompasses at least part of the delta globin gene. In an exemplary embodiment, the HPFH region is a region of the promoter of HBG1. In an exemplary embodiment, the HPFH region is a region of the promoter of HBG2. In an exemplary embodiment, the HPFH region is a region described in Sankaran V G et al. NEJM (2011) 365:807-814. In an exemplary embodiment, the HPFH region is the French breakpoint deletional HPFH as described in Sankaran V G et al. NEJM (2011) 365:807-814. In an exemplary embodiment, the HPFH region is the Algerian HPFH as described in Sankaran V G et al. NEJM (2011) 365:807-814. In an exemplary embodiment, the HPFH region is the Sri Lankan HPFH as described in Sankaran V G et al. NEJM (2011) 365:807-814. In an exemplary embodiment, the HPFH region is the HPFH-3 as described in Sankaran V G et al. NEJM (2011) 365:807-814. In an exemplary embodiment, the HPFH region is the HPFH-2 as described in Sankaran V G et al. NEJM (2011) 365:807-814. In an embodiment, the HPFH-1 region is the HPFH-3 as described in Sankaran V G et al. NEJM (2011) 365:807-814. In an exemplary embodiment, the HPFH region is the Sri Lankan (δβ)⁰-thalassemia HPFH as described in Sankaran V G et al. NEJM (2011) 365:807-814. In an exemplary embodiment, the HPFH region is the Sicilian (δβ)⁰-thalassemia HPFH as described in Sankaran V G et al. NEJM (2011) 365:807-814. In an exemplary embodiment, the HPFH region is the Macedonian (δβ)⁰-thalassemia HPFH as described in Sankaran V G et al. NEJM (2011) 365:807-814. In an exemplary embodiment, the HPFH region is the Kurdish β⁰-thalassemia HPFH as described in Sankaran V G et al. NEJM (2011) 365:807-814. In an exemplary embodiment, the HPFH region is the region located at Chr11:5213874-5214400 (hg18). In an exemplary embodiment, the HPFH region is the region located at Chr11:5215943-5215046 (hg18). In an exemplary embodiment, the HPFH region is the region located at Chr11:5234390-5238486 (hg38). The term “Nondeletional HPFH” refers to a mutation that does not comprise an insertion or deletion of one or more nucleotides, which results in hereditary persistence of fetal hemoglobin, and is characterized in increased fetal hemoglobin in adult red blood cells. In exemplary embodiments, the nondeletional HPFH is a mutation described in Nathan and Oski's Hematology and Oncology of Infancy and Childhood, 8^{th} Ed., 2015, Orkin S H, Fisher D E, Look T, Lux S E, Ginsburg D, Nathan D G, Eds., Elsevier Saunders, the entire contents of which is incorporated herein by reference, for example the nondeletional HPFH mutations described at Table 21-5. Nondeletional HPFH regions include genomic sites which comprises or is near a nondeletional HPFH. In exemplary embodiments, the nondeletional HPFH region is the nucleic acid sequence of the HBG1 promoter region (Chr11:5,249,833 to Chr11:5,250,237, hg38; −strand), the nucleic acid sequence of the HBG2 promoter region (Chr11:5,254,738 to Chr11:5,255,164, hg38; −strand), or combinations thereof. In exemplary embodiments, the nondeletional HPFH region includes one or more of the nondeletional HPFH described in Nathan and Oski's Hematology and Oncology of Infancy and Childhood, 8^{th} Ed., 2015, Orkin S H, Fisher D E, Look T, Lux S E, Ginsburg D, Nathan D G, Eds., Elsevier Saunders (e.g., described in Table 21-5 therein). In exemplary embodiments, the nondeletional HPFH region is the nucleic acid sequence at chr11:5,250,094-5,250,237, −strand, hg38; or the nucleic acid sequence at chr11:5,255,022-5,255,164, −strand, hg38; or the nucleic acid sequence at chr11: 5,249,833-5,249,927, −strand, hg38; or the nucleic acid sequence at chr11: 5,254,738-5,254,851, −strand, hg38; or the nucleic acid sequence at chr11:5,250,139-5,250,237, −strand, hg38; or combinations thereof.

“BCL11a enhancer” as the term is used herein, refers to nucleic acid sequence which affects, e.g., enhances, expression or function of BCL11a. See e.g., Bauer et al., Science, vol. 342, 2013, pp. 253-257. The BCL11a enhancer may be, for example, operative only in certain cell types, for example, cells of the erythroid lineage. One example of a BCL11a enhancer is the nucleic acid sequence between exon 2 and exon 3 of the BCL11a gene gene (e.g., the nucleic acid at or corresponding to positions +55: Chr2:60497676-60498941; +58: Chr2:60494251-60495546; +62: Chr2:60490409-60491734 as recorded in hg38). In an embodiment, the BCL11a Enhancer is the +62 region of the nucleic acid sequence between exon 2 and exon 3 of the BCL11a gene. In an embodiment, the BCL11a Enhancer is the +58 region of the nucleic acid sequence between exon 2 and exon 3 of the BCL11a gene. In an embodiment, the BCL11a Enhancer is the +55 region of the nucleic acid sequence between exon 2 and exon 3 of the BCL11a gene.

The terms “hematopoietic stem and progenitor cell” or “HSPC” are used interchangeably, and refer to a population of cells comprising both hematopoietic stem cells (“HSCs”) and hematopoietic progenitor cells (“HPCs”). Such cells are characterized, for example, as CD34+. In exemplary embodiments, HSPCs are isolated from bone marrow. In other exemplary embodiments, HSPCs are isolated from peripheral blood. In other exemplary embodiments, HSPCs are isolated from umbilical cord blood.

“AAVS1” refers to the genomic location at ch19:50,900,000-58,617,616 according to hg38. The terms “hematopoietic stem and progenitor cell” or “HSPC” are used interchangeably, and refer to a population of cells comprising both hematopoietic stem cells (“HSCs”) and hematopoietic progenitor cells (“HPCs”). Such cells are characterized, for example, as CD34+. In exemplary embodiments, HSPCs are isolated from bone marrow. In other exemplary embodiments, HSPCs are isolated from peripheral blood. In other exemplary embodiments, HSPCs are isolated from umbilical cord blood.

The term “Hematopoietic progenitor cells” (HPCs) as used herein refers to primitive hematopoietic cells that have a limited capacity for self-renewal and the potential for multilineage differentiation (e.g., myeloid, lymphoid), mono-lineage differentiation (e.g., myeloid or lymphoid) or cell-type restricted differentiation (e.g., erythroid progenitor) depending on placement within the hematopoietic hierarchy (Doulatov et al., Cell Stem Cell 2012).

“Hematopoietic stem cells” (HSCs) as used herein refer to immature blood cells having the capacity to self-renew and to differentiate into more mature blood cells comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages). HSCs are interchangeably described as stem cells throughout the specification. It is known in the art that such cells may or may not include CD34+ cells. CD34+ cells are immature cells that express the CD34 cell surface marker. CD34+ cells are believed to include a subpopulation of cells with the stem cell properties defined above. It is well known in the art that HSCs are multipotent cells that can give rise to primitive progenitor cells (e.g., multipotent progenitor cells) and/or progenitor cells committed to specific hematopoietic lineages (e.g., lymphoid progenitor cells). The stem cells committed to specific hematopoietic lineages may be of T cell lineage, B cell lineage, dendritic cell lineage, Langerhans cell lineage and/or lymphoid tissue-specific macrophage cell lineage. In addition, HSCs also refer to long term HSC (LT-HSC) and short term HSC (ST-HSC). ST-HSCs are more active and more proliferative than LT-HSCs. However, LT-HSC have unlimited self renewal (i.e., they survive throughout adulthood), whereas ST-HSC have limited self renewal (i.e., they survive for only a limited period of time). Any of these HSCs can be used in any of the methods described herein. Optionally, ST-HSCs are useful because they are highly proliferative and thus, quickly increase the number of HSCs and their progeny. Hematopoietic stem cells are optionally obtained from blood products. A blood product includes a product obtained from the body or an organ of the body containing cells of hematopoietic origin. Such sources include un-fractionated bone marrow, umbilical cord, peripheral blood (e.g., mobilized peripheral blood, e.g., mobilized with a mobilization agent such as G-CSF or Plerixafor® (AMD3100)), liver, thymus, lymph and spleen. All of the aforementioned crude or un-fractionated blood products can be enriched for cells having hematopoietic stem cell characteristics in ways known to those of skill in the art. In an embodiment, HSCs are characterized as CD34+/CD38−/CD90+/CD45RA−. In embodiments, the HSC s are characterized as CD34+/CD90+/CD49f+ cells.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be synthesized or can be derived from a biological sample, or might be macromolecule besides a polypeptide. Such a biological sample can include, but is not limited to a tissue sample, a cell or a fluid with other biological components.

The term “autologous” refers to any material derived from the same individual into whom it is later to be re-introduced.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically

The term “xenogeneic” refers to a graft derived from an animal of a different species. “Derived from” as that term is used herein, indicates a relationship between a first and a second molecule. It generally refers to structural similarity between the first molecule and a second molecule and does not connotate or include a process or source limitation on a first molecule that is derived from a second molecule.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses.

Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

As used herein in connection with a messenger RNA (mRNA), a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000 (SEQ ID NO: 508), preferably greater than 64, more preferably greater than 100, most preferably greater than 300 or 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a disorder, e.g., a hemoglobinopathy, or the amelioration of one or more symptoms (preferably, one or more discernible symptoms) of a disorder, e.g., a hemoglobinopathy, resulting from the administration of one or more therapies (e.g., one or more therapeutic agents such as a gRNA molecule, CRISPR system, or modified cell of the invention). In specific embodiments, the terms “treat”, “treatment” and “treating” refer to the amelioration of at least one measurable physical parameter of a hemoglobinopathy disorder, not discernible by the patient. In other embodiments the terms “treat”, “treatment” and “treating” refer to the inhibition of the progression of a disorder, either physically by, e.g., stabilization of a discernible symptom, physiologically by, e.g., stabilization of a physical parameter, or both. In other embodiments the terms “treat”, “treatment” and “treating” refer to the reduction or stabilization of a symptom of a hemoglobinopathy, e.g., sickle cell disease or beta-thalassemia.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid and/or protein is transferred or introduced into the host cell. A “transfected” or “transformed” or “transduced” cell is one which has been transfected, transformed or transduced with exogenous nucleic acid and/or protein. The cell includes the primary subject cell and its progeny.

The term “specifically binds,” refers to a molecule recognizing and binding with a binding partner (e.g., a protein or nucleic acid) present in a sample, but which molecule does not substantially recognize or bind other molecules in the sample.

The term “bioequivalent” refers to an amount of an agent other than the reference compound, required to produce an effect equivalent to the effect produced by the reference dose or reference amount of the reference compound.

“Refractory” as used herein refers to a disease, e.g., a hemoglobinopathy, that does not respond to a treatment. In embodiments, a refractory hemoglobinopathy can be resistant to a treatment before or at the beginning of the treatment. In other embodiments, the refractory hemoglobinopathy can become resistant during a treatment. A refractory hemoglobinopathy is also called a resistant hemoglobinopathy.

“Relapsed” as used herein refers to the return of a disease (e.g., hemoglobinopathy) or the signs and symptoms of a disease such as a hemoglobinopathy after a period of improvement, e.g., after prior treatment of a therapy, e.g., hemoglobinopathy therapy.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

Gene Editing Systems

As used herein, the term “gene editing system” refers to a system comprising one or more DNA-binding domains or components and one or more DNA-modifying domains or components, or isolated nucleic acids, e.g., one or more vectors, encoding said DNA-binding and DNA-modifying domains or components. Gene editing systems are used, for example, for modifying the nucleic acid of a target gene and/or for modulating the expression of a target gene. In gene editing systems, for example, the one or more DNA-binding domains or components are associated with the one or more DNA-modifying domains or components, such that the one or more DNA-binding domains target the one or more DNA-modifying domains or components to a specific nucleic acid site.

Gene editing systems include but are not limited to, zinc finger nucleases, transcription activator-like effector nucleases (TALENs); clustered regularly interspaced short palindromic repeats (CRISPR)/Cas systems, and meganuclease systems. Without wishing to be bound by theory, it is believed that the known gene editing systems may exhibit unwanted DNA-modifying activity which is detrimental to their utility in therapeutic applications. These concerns are particularly apparent in the use of gene editing systems for in vivo modification of genes or gene expression, e.g., where cells are engineered to constitutively express components of a gene editing system, such as through lentiviral or adenoviral vector transfection.

CRISPR Gene Editing Systems

“CRISPR” as used herein refers to a set of clustered regularly interspaced short palindromic repeats, or a system comprising such a set of repeats. “Cas,” as used herein, refers to a CRISPR-associated protein. The diverse CRISPR-Cas systems can be divided into two classes according to the configuration of their effector modules: class 1 CRISPR systems utilize several Cas proteins and the crRNA to form an effector complex, whereas class 2 CRISPR systems employ a large single-component Cas protein in conjunction with crRNAs to mediate interference. One example of class 2 CRISPR-Cas system employs Cpf1 (CRISPR from Prevotella and Francisella 1). See, e.g., Zetsche et al., Cell 163:759-771 (2015), the content of which is herein incorporated by reference in its entirety. The term “Cpf1” as used herein includes all orthologs, and variants that can be used in a CRISPR system. The present invention provides compositions and methods of treatment using gene editing systems, for example, CRISPR systems described herein.

Naturally-occurring CRISPR systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

The CRISPR system has been modified for use in gene editing (silencing, enhancing or changing specific genes) in eukaryotes such as mice, primates and humans. Wiedenheft et al. (2012) Nature 482: 331-8. This is accomplished by, for example, introducing into the eukaryotic cell one or more vectors encoding a specifically engineered guide RNA (gRNA) (e.g., a gRNA comprising sequence complementary to sequence of a eukaryotic genome) and one or more appropriate RNA-guided nucleases, e.g., Cas proteins. The RNA guided nuclease forms a complex with the gRNA, which is then directed to the target DNA site by hybridization of the gRNA's sequence to complementary sequence of a eukaryotic genome, where the RNA-guided nuclease then induces a double or single-strand break in the DNA. Insertion or deletion of nucleotides at or near the strand break creates the modified genome.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stem et al. (2010) Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

In some embodiments, the RNA-guided nuclease is a Cas molecule, e.g., a Cas9 molecule. A “Cas9 molecule,” as used herein, refers to a molecule that can interact with a gRNA molecule (e.g., sequence of a domain of a tracr) and, in concert with the gRNA molecule, localize (e.g., target or home) to a site which comprises a target sequence and PAM sequence.

According to the present invention, Cas9 molecules of, derived from, or based on the Cas9 proteins of a variety of species can be used in the methods and compositions described herein. For example, Cas9 molecules of, derived from, or based on, e.g., S. pyogenes, S. thermophilus, Staphylococcus aureus and/or Neisseria meningitidis Cas9 molecules, can be used in the systems, methods and compositions described herein. Additional Cas9 species include: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

In some embodiments, the ability of an active Cas9 molecule to interact with and cleave a target nucleic acid is PAM sequence dependent. A PAM sequence is a sequence in the target nucleic acid. In an embodiment, cleavage of the target nucleic acid occurs upstream from the PAM sequence. Active Cas9 molecules from different bacterial species can recognize different sequence motifs (e.g., PAM sequences). In an embodiment, an active Cas9 molecule of S. pyogenes recognizes the sequence motif NGG and directs cleavage of a target nucleic acid sequence 1 to 10, e.g., 3 to 5, base pairs upstream from that sequence. See, e.g., Mali el al, SCIENCE 2013; 339(6121): 823-826. In an embodiment, an active Cas9 molecule of S. thermophilus recognizes the sequence motif NGGNG and NNAG AAW (W=A or T) and directs cleavage of a core target nucleic acid sequence 1 to 10, e.g., 3 to 5, base pairs upstream from these sequences. See, e.g., Horvath et al., SCIENCE 2010; 327(5962): 167-170, and Deveau et al, J BACTERIOL 2008; 190(4): 1390-1400. In an embodiment, an active Cas9 molecule of S. mutans recognizes the sequence motif NGG or NAAR (R-A or G) and directs cleavage of a core target nucleic acid sequence 1 to 10, e.g., 3 to 5 base pairs, upstream from this sequence. See, e.g., Deveau et al., J BACTERIOL 2008; 190(4): 1390-1400.

In an embodiment, an active Cas9 molecule of S. aureus recognizes the sequence motif NNGRR (R=A or G) and directs cleavage of a target nucleic acid sequence 1 to 10, e.g., 3 to 5, base pairs upstream from that sequence. See, e.g., Ran F. et al., NATURE, vol. 520, 2015, pp. 186-191. In an embodiment, an active Cas9 molecule of N. meningitidis recognizes the sequence motif NNNNGATT and directs cleavage of a target nucleic acid sequence 1 to 10, e.g., 3 to 5, base pairs upstream from that sequence. See, e.g., Hou et al., PNAS EARLY EDITION 2013, 1-6. The ability of a Cas9 molecule to recognize a PAM sequence can be determined, e.g., using a transformation assay described in Jinek et al, SCIENCE 2012, 337:816.

Exemplary naturally occurring Cas9 molecules are described in Chylinski et al, RNA Biology 2013; 10:5, 727-737. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 1 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. pyogenes (e.g., strain SF370, MGAS 10270, MGAS 10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA 159, NN2025), S. macacae (e.g., strain NCTC1 1558), S. gallolylicus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. cmginosus (e.g.; strain F0211), S. agalactia* (e.g., strain NEM316, A909), Listeria monocytogenes (e.g., strain F6854), Listeria innocua (L. innocua, e.g., strain Clip 11262), Enterococcus italicus (e.g., strain DSM 15952), or Enterococcus faecium (e.g., strain 1,23,408). Additional exemplary Cas9 molecules are a Cas9 molecule of Neisseria meningitidis (Hou et al. PNAS Early Edition 2013, 1-6) and a S. aureus Cas9 molecule.

In an embodiment, a Cas9 molecule, e.g., an active Cas9 molecule or inactive Cas9 molecule, comprises an amino acid sequence: having 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; any Cas9 molecule sequence described herein or a naturally occurring Cas9 molecule sequence, e.g., a Cas9 molecule from a species listed herein or described in Chylinski et al., RNA Biology 2013, 10:5, ‘I2’I-T,1 Hou et al. PNAS Early Edition 2013, 1-6.

In an embodiment, a Cas9 molecule comprises an amino acid sequence having 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; S. pyogenes Cas9 (UniProt Q99ZW2). In embodiments, the Cas9 molecule is a S. pyogenes Cas9 variant, such as a variant described in Slaymaker et al., Science Express, available online Dec. 1, 2015 at Science DOI: 10.1126/science.aad5227; Kleinstiver et al., Nature, 529, 2016, pp. 490-495, available online Jan. 6, 2016 at doi: 10.1038/nature16526; or US2016/0102324, the contents of which are incorporated herein in their entirety. In an embodiment, the Cas9 molecule is catalytically inactive, e.g., dCas9. Tsai et al. (2014), Nat. Biotech. 32:569-577; U.S. Pat. Nos. 8,871,445; 8,865,406; 8,795,965; 8,771,945; and 8,697,359, the contents of which are hereby incorporated by reference in their entirety. A catalytically inactive Cas9, e.g., dCas9, molecule may be fused with a transcription modulator, e.g., a transcription repressor or transcription activator.

In an embodiment, the Cas9 molecule of the invention can be any of the Cas9 variants, including chimeric Cas9 molecules, described in, e.g., U.S. Pat. Nos. 8,889,356, 8,889,418, 8,932,814, WO2016022363, US20150118216, WO2014152432, US20140295556, US2016153003, U.S. Pat. Nos. 9,322,037, 9,388,430, WO2015089406, U.S. Pat. No. 9,267,135, WO2015006294, WO2016106244, WO2016057961, WO2016131009, and WO2017115268, the content of which are hereby incorporated by reference in their entirety.

In some embodiments, the Cas9 molecule, e.g., a Cas9 of S. pyogenes, may additionally comprise one or more amino acid sequences that confer additional activity. In some aspects, the Cas9 molecule may comprise one or more nuclear localization sequences (NLSs), such as at least 1, 2, 3, 4, 5, 6, 7, 8, 9, 10, or more NLSs. Typically, an NLS consists of one or more short sequences of positively charged lysines or arginines exposed on the protein surface, but other types of NLS are known. Non-limiting examples of NLSs include an NLS sequence comprising or derived from: the NLS of the SV40 virus large T-antigen, having the amino acid sequence PKKKRKV (SEQ ID NO: 509). Other suitable NLS sequences are known in the art (e.g., Sorokin, Biochemistry (Moscow) (2007) 72:13, 1439-1457; Lange J Biol Chem. (2007) 282:8, 5101-5). In any of the aforementioned embodiments, the Cas9 molecule may additionally (or alternatively) comprise a tag, e.g., a His tag, e.g., a His(6) tag (SEQ ID NO: 510) or His(8) tag (SEQ ID NO: 511), e.g., at the N terminus or the C terminus.

Thus, engineered CRISPR gene editing systems, e.g., for gene editing in eukaryotic cells, typically involve (1) a guide RNA molecule (gRNA) comprising a targeting domain (which is capable of hybridizing to the genomic DNA target sequence), and sequence which is capable of binding to a Cas, e.g., Cas9 enzyme, and (2) a Cas, e.g., Cas9, protein. This second domain may comprise a domain referred to as a tracr domain. The targeting domain and the sequence which is capable of binding to a Cas, e.g., Cas9 enzyme, may be disposed on the same (sometimes referred to as a single gRNA, chimeric gRNA or sgRNA) or different molecules (sometimes referred to as a dual gRNA or dgRNA). If disposed on different molecules, each includes a hybridization domain which allows the molecules to associate, e.g., through hybridization.

gRNA molecule formats are known in the art. An exemplary gRNA molecule, e.g., dgRNA molecule, of the present invention comprises, e.g., consists of, a first nucleic acid having the sequence:

(SEQ ID NO: 512)
nnnnnnnnnnnnnnnnnnnnGUUUUAGAGCUAUGCUGUUUUG,

where the “n”'s refer to the residues of the targeting domain, e.g., as described herein, and may consist of 15-25 nucleotides, e.g., consists of 20 nucleotides;

and a second nucleic acid sequence having the exemplary sequence: AACUUACCAAGGAACAGCAUAGCAAGUUAAAAUAAGGCUAGUCCGUUAUCAACUUGAAAAAGUGGCACCGAGUCGGUGC (SEQ ID NO: 513), optionally with 1, 2, 3, 4, 5, 6, or 7 (e.g., 4 or 7, e.g., 7) additional U nucleotides at the 3′ end.

The second nucleic acid molecule may alternatively consist of a fragment of the sequence above, wherein such fragment is capable of hybridizing to the first nucleic acid. An example of such second nucleic acid molecule is: AACAGCAUAGCAAGUUAAAAUAAGGCUAGUCCGUUAUCAACUUGAAAAAGUGGCACCGAGUCGGUGC (SEQ ID NO: 514), optionally with 1, 2, 3, 4, 5, 6, or 7 (e.g., 4 or 7, e.g., 7) additional U nucleotides at the 3′ end.

Another exemplary gRNA molecule, e.g., a sgRNA molecule, of the present invention comprises, e.g., consists of a first nucleic acid having the sequence: nnnnnnnnnnnnnnnnnnnGUUUUAGAGCUAGAAAUAGCAAGUUAAAAUAAGGCUAGUCCGUUAUCAACUUGAAAAAGUGGCACCGAGUCGGUGC (SEQ ID NO: 515), where the “n”'s refer to the residues of the targeting domain, e.g., as described herein, and may consist of 15-25 nucleotides, e.g., consist of 20 nucleotides, optionally with 1, 2, 3, 4, 5, 6, or 7 (e.g., 4 or 7, e.g., 4) additional U nucleotides at the 3′ end.

Additional components and/or elements of CRISPR gene editing systems known in the art, e.g., are described in U.S. Publication No. 2014/0068797, WO2015/048577, and Cong (2013) Science 339: 819-823, the contents of which are hereby incorporated by reference in their entirety. Such systems can be generated which inhibit a target gene, by, for example, engineering a CRISPR gene editing system to include a gRNA molecule comprising a targeting domain that hybridizes to a sequence of the target gene. In embodiments, the gRNA comprises a targeting domain which is fully complementarity to 15-25 nucleotides, e.g., 20 nucleotides, of a target gene. In embodiments, the 15-25 nucleotides, e.g., 20 nucleotides, of the target gene, are disposed immediately 5′ to a protospacer adjacent motif (PAM) sequence recognized by the RNA-guided nuclease, e.g., Cas protein, of the CRISPR gene editing system (e.g., where the system comprises a S. pyogenes Cas9 protein, the PAM sequence comprises NGG, where N can be any of A, T, G or C).

In some embodiments, the gRNA molecule and RNA-guided nuclease, e.g., Cas protein, of the CRISPR gene editing system can be complexed to form a RNP complex. Such RNP complexes may be used in the methods and apparatus described herein. In other embodiments, nucleic acid encoding one or more components of the CRISPR gene editing system may be used in the methods and apparatus described herein.

In some embodiments, foreign DNA can be introduced into the cell along with the CRISPR gene editing system, e.g., DNA encoding a desired transgene, with or without a promoter active in the target cell type. Depending on the sequences of the foreign DNA and target sequence of the genome, this process can be used to integrate the foreign DNA into the genome, at or near the site targeted by the CRISPR gene editing system. For example, 3′ and 5′ sequences flanking the transgene may be included in the foreign DNA which are homologous to the gene sequence 3′ and 5′ (respectively) of the site in the genome cut by the gene editing system. Such foreign DNA molecule can be referred to “template DNA.”

In an embodiment, the CRISPR gene editing system of the present invention comprises Cas9, e.g., S. pyogenes Cas9, and a gRNA comprising a targeting domain which hybridizes to a sequence of a gene of interest. In an embodiment, the gRNA and Cas9 are complexed to form a RNP. In an embodiment, the CRISPR gene editing system comprises nucleic acid encoding a gRNA and nucleic acid encoding a Cas protein, e.g., Cas9, e.g., S. pyogenes Cas9. In an embodiment, the CRISPR gene editing system comprises a gRNA and nucleic acid encoding a Cas protein, e.g., Cas9, e.g., S. pyogenes Cas9.

In some embodiments, inducible control over Cas9 and sgRNA expression can be utilized to optimize efficiency while reducing the frequency of off-target effects thereby increasing safety. Examples include, but are not limited to, transcriptional and post-transcriptional switches listed as follows; doxycycline inducible transcription Loew et al. (2010) BMC Biotechnol. 10:81, Shield inducible protein stabilization Banaszynski et al. (2016) Cell 126: 995-1004, Tamoxifen induced protein activation Davis et al. (2015) Nat. Chem. Biol. 11: 316-318, Rapamycin or optogenetic induced activation or dimerization of split Cas9 Zetsche (2015) Nature Biotechnol. 33(2): 139-142, Nihongaki et al. (2015) Nature Biotechnol. 33(7): 755-760, Polstein and Gersbach (2015) Nat. Chem. Biol. 11: 198-200, and SMASh tag drug inducible degradation Chung et al. (2015) Nat. Chem. Biol. 11: 713-720.

With respect to general information on CRISPR-Cas Systems, components thereof, and delivery of such components, including methods, materials, delivery vehicles, vectors, particles, AAV, and making and using thereof including as to amounts and formulations, all useful in the practice of the instant invention, reference is made to: U.S. Pat. Nos. 8,697,359, 8,771,945, 8,795,965, 8,865,406, 8,871,445, 8,889,356, 8,889,418 and 8,895,308; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458). US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035). US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); European Patent Applications EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/05148), WO 2014/204723 (PCT/US2014/041790), WO 2014/204724 (PCT/US2014/041800), WO 2014/204725 (PCT/US2014/041803), WO 2014/204726 (PCT US2014/041804), WO 2014/204727 (PCT US2014/041806), WO 2014/204728 (PCT/US2014/041808), and WO 2014/204729 (PCT US2014/041809). Reference is also made to U.S. provisional patent applications 1/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/836,127, 61/836,101, 61/836,080 and 61/835,973, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent applications Nos: PCT/US2014/041803, PCT/US2014/041800, PCT/US2014/041809, PCT/US2014/041804 and PCT US2014/041806, each filed Jun. 10, 2014 6/10/14; PCT US2014/041808 filed Jun. 11, 2014; and PCT/US2014/62558 filed Oct. 28, 2014, and U.S. Provisional Patent Applications Ser. Nos. 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. provisional patent applications Nos. 62/055,484, 62/055,460, and 62/055,487, filed Sep. 25, 2014; U.S. provisional patent application 61/980,012, filed Apr. 15, 2014; and U.S. provisional patent application 61/939,242 filed Feb. 12, 2014. Reference is made to PCT application designating, inter alia, the United States, application No. PCT/US 14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. Reference is made to U.S. provisional patent application U.S. Ser. No. 61/980,012 filed Apr. 15, 2014. Reference is made to PCT application designating, inter alia, the United States, application No. PCT/US 14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. [0054] Mention is also made of U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. application 62/091,462, 12 Dec. 2014, DEAD GUIDES FOR CRISPR TRANSCRIPTION FACTORS; U.S. application 62/096,324, 23 Dec. 2014, DEAD GUIDES FOR CRISPR TRANSCRIPTION FACTORS; U.S. application 62/091,456, 12 Dec. 2014, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, RNA-TARGETING SYSTEM; U.S. application 62/096,656, 24 Dec. 2014, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/054,675, 24 Sep. 2014, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

Also with respect to general information on CRISPR-Cas Systems, mention is made of the following (also hereby incorporated herein by reference):
Multiplex genome engineering using CRISPR/Cas systems, Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013); One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013); Optical control of mammalian endogenous transcription and epigenetic states. Konernann S, Brigham M D, Trevino A E, Hsu P D, Heidenreich M, Cong L, Piatt R J, Scott D A, Church G M, Zhang F. Nature. 2013 Aug. 22; 500(7463):472-6. doi: 10.1038/Nature 12466. Epub 2013 Aug. 23;
Double Nicking by RNA-Guided CRISPR Cas9 for Enhanced Genome Editing Specificity. Ran, F A., Hsu, P D., Lin, C Y., Gootenberg, J S., Konernann, S., Trevino, A E, Scott, D A., Inoue, A., Matoba, S., Zhang, Y, & Zhang, F. Cell August 28. pii: S0092-8674(13)01015-5. (2013
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L. A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308. (2013); Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science Dec. 12. (2013). [Epub ahead of print]; Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell Feb. 27. (2014). 156(5):935-49;
Genome-wide binding of the CRISPR endonuclease Cas9 in mammalian cells. Wu X., Scott D A., Kriz A J., Chiu A C, Hsu P D., Dadon D B., Cheng A W., Trevino A E., Konermann S., Chen S., Jaenisch R., Zhang F., Sharp P A. Nat Biotechnol. (2014) Apr. 20. doi: 10.1038/nbt.2889,
CRISPR-Cas9 Knockin Mice for Genome Editing and Cancer Modeling, Piatt et al., Cell 159(2): 440-455 (2014) DOI: 10.1016/j.cell.2014.09.014,
Development and Applications of CRISPR-Cas9 for Genome Engineering, Hsu et al. Cell 157, 1262-1278 (Jun. 5, 2014) (Hsu 2014),
Genetic screens in human cells using the CRISPR/Cas9 system, Wang et al., Science. 2014 Jan. 3; 343(6166): 80-84. doi: 10.1126/science.1246981,
Rational design of highly active sgRNAs for CRISPR-Cas9-mediated gene inactivation, Doench et al., Nature Biotechnology published online 3 Sep. 2014; doi: 10.1038/nbt.3026, and In vivo interrogation of gene function in the mammalian brain using CRISPR-Cas9, Swiech et al, Nature Biotechnology; published online 19 Oct. 2014; doi:10.1038/nbt.3055.

Genome-scale transcriptional activation by an engineered CRISPR-Cas9 complex, onermann S, Brigham M D, Trevino A E, Joung J, Abudavyeh 00, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2): 139-42 (2015);
Genome-wide CRISPR Screen in a Mouse Model of Tumor Growth and Metastasis, Chen S, Sanjana N E, Zheng, Shalem O, Lee, Shi X, Scott D A, Song J, Pan J Q, Weissleder R, Lee H, Zhang F, Sharp P A, Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, oonin E V, Sharp P A, Zhang F., (published online 1 Apr. 2015), Nature. April 9; 520(7546): 186-91 (2015)
High-throughput functional genomics using CRISPR-Cas9, Shalem et al, Nature Reviews Genetics 16, 299-311 (May 2015).
Sequence determinants of improved CRISPR sgRNA design, Xu et al., Genome Research 25, 1 147-1 157 (August 2015).
A Genome-wide CRISPR Screen in Primary Immune Cells to Dissect Regulatory Networks, Parnas et al., Cell 162, 675-686 (Jul. 30, 2015).
CRISPR/Cas9 cleavage of viral DNA efficiently suppresses hepatitis B virus, Ramanan et al., Scientific Reports 5:10833. doi: 10.1038/srep10833 (Jun. 2, 2015).
Crystal Structure of Staphylococcus aureus Cas9, Nishimasu et al., Cell 162, 1113-1126 (Aug. 27, 2015).
BCL 11 A enhancer dissection by Cas9-mediated in situ saturating mutagenesis, Canver et al., Nature 527(7577): 192-7 (Nov. 12, 2015) doi: 10.1038/nature15521. Epub 2015 Sep. 16. each of which is incorporated herein by reference, and discussed briefly below:

Cong et al. engineered type II CRISPR/Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple targeting domains can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR/Cas system can be further improved to increase its efficiency and versatility.

Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems, The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.

Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epistatic gene interactions.

Konernann et al. addressed the need in the art for versatile and robust technologies that enable optical and chemical modulation of DNA-binding domains based CRISPR Cas9 enzyme and also Transcriptional Activator Like Effectors.

Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a gRNA's targeting domain, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity. Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.

Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.

Shaiem et al. described a new way to interrogate gene function on a genome-wide scale.

Their studies showed that delivery of a genome-scale CRISPR-Cas9 knockout (GeC O) library targeted 18,080 genes with 64,751 unique gRNA molecules enabled both negative and positive selection screening in human cells, First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED 12 as well as novel hits NF2, CUL3, TADA2B, and TADAL The authors observed a high level of consistency between independent guide RNAs targeting the same gene and a high rate of hit confirmation, and thus demonstrated the promise of genome-scale screening with Cas9.

Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.

Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive paining with target DNA is required for cleavage.

Piatt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.

Hsu et al. (2014) is a review article that discusses generally CRISPR-Cas9 history from yogurt to genome editing, including genetic screening of cells.

Wang et al, (2014) relates to a pooled, loss-of-function genetic screening approach suitable for both positive and negative selection that uses a genome-scale lentiviral single guide RNA (sgRNA) library.

Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an online tool for designing sgRNAs.

Swiech et al. demonstrate that AAV-mediated SpCas9 genome editing can enable reverse genetic studies of gene function in the brain.

Konermann et al. (2015) discusses the ability to attach multiple effector domains, e.g., transcriptional activator, functional and epigenomic regulators at appropriate positions on the guide such as stem or tetraloop with and without linkers.

Zetsche et al. demonstrates that the Cas9 enzyme can be split into two and hence the assembly of Cas9 for activation can be controlled.

Chen et al relates to multiplex screening by demonstrating that a genome-wide in vivo CRISPR-Cas9 screen in mice reveals genes regulating lung metastasis. >Ran et al. (2015) relates to SaCas9 and its ability to edit genomes and demonstrates that one cannot extrapolate from biochemical assays. Shalem et al. (2015) described ways in which catalytically inactive Cas9 (dCas9) fusions are used to synthetically repress (CRISPRi) or activate (CRISPRa) expression, showing, advances using Cas9 for genome-scale screens, including arrayed and pooled screens, knockout approaches that inactivate genomic loci and strategies that modulate transcriptional activity.

Shalem et al. (2015) described ways in which catalytically inactive Cas9 (dCas9) fusions are used to synthetically repress (CRISPRi) or activate (CRISPRa) expression, showing, advances using Cas9 for genome-scale screens, including arrayed and pooled screens, knockout approaches that inactivate genomic loci and strategies that modulate transcriptional activity.

Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR Cas9 knockout.

Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of TIr4 signaling and previously unknown candidates were identified and classified into three functional modules with distinct effects on the canonical responses to LPS.

Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA. Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.

Slaymaker et al (2015) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.

Tsai et al, “Dimeric CRISPR A-guided Fok1 nucleases for highly specific genome editing,” Nature Biotechnology 32(6): 569-77 (2014) which is not believed to be prior art to the instant invention or application, but which may be considered in the practice of the instant invention. Mention is also made of Konermann et al., “Genome-scale transcription activation by an engineered CRISPR-Cas9 complex,” doi: 10.1038/nature14136, incorporated herein by reference.

In general, the CRISPR-Cas or CRISPR system is as used in the foregoing documents, such as WO 2014/093622 (PCT/US2013/074667) and refers collectively to transcripts and other elements involved in the expression of or directing the activity of CRISPR-associated (“Cas”) genes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a targeting domain is designed to have complementarity, where hybridization between a target sequence and a targeting promotes the formation of a CRISPR complex. A target sequence may comprise any polynucleotide, such as DNA or RNA polynucleotides. In some embodiments, a target sequence is located in the nucleus or cytoplasm of a cell. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used. In some embodiments it may be preferred in a CRISPR complex that the tracr sequence has one or more hairpins and is 30 or more nucleotides in length, 40 or more nucleotides in length, or 50 or more nucleotides in length; the targeting domain is between 10 to 30 nucleotides in length, the CRISPR/Cas enzyme is a Type II Cas9 enzyme. In embodiments of the invention the terms guide sequence and targeting domain are used interchangeably as in foregoing cited documents such as WO 2014/093622 (PCT US2013/074667). In general, a targeting domain is any polynucleotide sequence having sufficient complementarity with a target polynucleotide sequence to hybridize with the target sequence and direct sequence-specific binding of a CRISPR complex to the target sequence. In some embodiments, the degree of complementarity between a targeting domain and its corresponding target sequence, when optimally aligned using a suitable alignment algorithm, is about or more than about 50%, 60%, 75%, 80%, 85%, 90%, 95%, 97.5%, 99%, or more. Preferrably the targeting domain is 100% complementary (fully complementary) to the target sequence. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and aq (available at maq.sourceforge.net). In some embodiments, a targeting domain is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a targeting domain is less than about 75, 50, 45, 40, 35, 30, 25, 20, 15, 12, or fewer nucleotides in length. Preferably the targeting domain is 10-30 nucleotides long. The ability of a targeting domain to direct sequence-specific binding of a CRISPR complex to a target sequence may be assessed by any suitable assay. For example, the components of a CRISPR system sufficient to form a CRISPR complex, including the targeting domain to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the targeting domain to be tested and a control targeting domain different from the test targeting domain, and comparing binding or rate of cleavage at the target sequence between the test and control targeting domain reactions. Other assays are possible, and will occur to those skilled in the art. A targeting domain may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMNNNNNNNNNNNNXGG where NNNNNNNN XGG (N is A, G, T, or C; and X can be anything) has a single occurrence in the genome. A unique target sequence in a genome may include an S. pyogenes Cas9 target site of the form MMM MMMMMNNNNNNNNNNNXGG where NNNNXGG (N is A, G, T, or C; and X can be anything) has a single occurrence in the genome. For the S. thermophilus CRISPR Cas9, a unique target sequence in a genome may include a Cas9 target site of the form MMMMMMMMNNNNNNXXAGAAW (SEQ ID NO: 516) where NNNNNNXXAGAAW (SEQ ID NO: 517) (N is A, G, T, or C; X can be anything; and W is A or T) has a single occurrence in the genome. A unique target sequence in a genome may include an S. thermophilus CRISPR1 Cas9 target site of the form MMMMMMMNNNNN NNXXAGAAW (SEQ ID NO: 518) where NNNNNNNNNNNXXAGAAW (SEQ ID NO: 519) (N is A, G, T, or C; X can be anything; and W is A or T) has a single occurrence in the genome. For the S. pyogenes Cas9, a unique target sequence in a genome may include a Cas9 target site of the form MMMMMMMMNNNNNNNNNXGGXG where NNNNNNNNNNNNXGGXG (N is A, G, T, or C; and X can be anything) has a single occurrence in the genome. A unique target sequence in a genome may include an S. pyogenes Cas9 target site of the form MMMMMMMMMNNNNNNNNNNXGGXG where NNNNNNNNNNNXGGXG (N is A, G, T, or C; and X can be anything) has a single occurrence in the genome. In each of these sequences may be A, G, T, or C, and need not be considered in identifying a sequence as unique. In some embodiments, a targeting domain is selected to reduce the degree secondary structure within the targeting domain. In some embodiments, about or less than about 75%, 50%, 40%, 30%, 25%, 20%, 15%, 10%, 5%, 1%, or fewer of the nucleotides of the targeting domain participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and P A Carr and G M Church, 2009, Nature Biotechnology 27(12): 1 151-62).

In some embodiments, the gRNA targeting domain is chosen to a sequence which affects a hemoglobinopathy. In embodiments, the gene editing system includes a CRISPR system including one or more gRNA molecules comprising a targeting domain complementary to any one of SEQ ID NO: 1 to 161,197 of PCT Publication WO2017/077394. In other embodiments, the gene editing system includes a CRISPR system including a gRNA molecule comprising a targeting domain complementary to any one of SEQ ID NO: 1 to 135 of PCT Publication WO2016/182917. In other embodiments, the gene editing system includes a CRISPR system including a gRNA molecule comprising a targeting domain according to any one of SEQ ID NO: 1232 to 1497, or a fragment thereof, of PCT Publication WO2017/115268. In other embodiments, the gene editing system includes a CRISPR system including a gRNA molecule comprising a targeting domain according to any one of SEQ ID NO: 86 to 181, or a fragment thereof, or SEQ ID NO: 1500 to 1595, or a fragment thereof, or SEQ ID NO: 1692 to 1761, or a fragment thereof, of PCT Publication WO2017/115268. In other embodiments, the gene editing system includes a CRISPR system including a gRNA molecule comprising a targeting domain according to any one of SEQ ID NO: 182 to 277, or a fragment thereof, or SEQ ID NO: 334 to 341, or a fragment thereof, of PCT Publication WO2017/115268. In other embodiments, the gene editing system includes a CRISPR system including a gRNA molecule comprising a targeting domain according to any one of SEQ ID NO: 278 to 333, or a fragment thereof, of PCT Publication WO2017/115268. In other embodiments, the gene editing system includes a CRISPR system including a gRNA molecule comprising a targeting domain according to any one of SEQ ID NO: 1596 to 1691, or a fragment thereof, of PCT Publication WO2017/115268.

Additional preferred gRNA targeting domain sequences, particularly for gene editing systems for treating hemoglobinopathies, are provided in Tables 1-3.

TABLE 1
Preferred Guide RNA Targeting Domains directed to the □Enhancer
Region of the BCL11a Gene (i.e., to a BCL11a Enhancer)

| | Exon/ | | | | SEQ |
|---|---|---|---|---|---|
| Id. | Feature | Strand | Targeting domain | Locations | ID NO: |
| CR00242 | 58 | + | UAUGCAAUUUUUGCCAAGAU | Chr2: 60494419-60494441 | 182 |
| CR00243 | 58 | + | UUUUGCCAAGAUGGGAGUAU | Chr2: 60494427-60494449 | 183 |
| CR00244 | 58 | + | UUUGCCAAGAUGGGAGUAUG | Chr2: 60494428-60494450 | 184 |
| CR00245 | 58 | + | UCAGUGAGAUGAGAUAUCAA | Chr2: 60494477-60494499 | 185 |
| CR00246 | 58 | + | CAGUGAGAUGAGAUAUCAAA | Chr2: 60494478-60494500 | 186 |
| CR00247 | 58 | + | CCAUCUCCCUAAUCUCCAAU | Chr2: 60494518-60494540 | 187 |
| CR00248 | 58 | − | CCAAUUGGAGAUUAGGGAGA | Chr2: 60494518-60494540 | 188 |
| CR00249 | 58 | − | GCUUUGCCAAUUGGAGAUUA | Chr2: 60494524-60494546 | 189 |
| CR00250 | 58 | − | GGCUUUGCCAAUUGGAGAUU | Chr2: 60494525-60494547 | 190 |
| CR00251 | 58 | + | AAUUGGCAAAGCCAGACUUG | Chr2: 60494535-60494557 | 191 |
| CR00252 | 58 | − | AGUCUGUAUUGCCCCAAGUC | Chr2: 60494546-60494568 | 192 |
| CR00253 | 58 | + | AGACUUGGGGCAAUACAGAC | Chr2: 60494548-60494570 | 193 |
| CR00255 | 58 | − | ACAUUUGGUGAUAAAUCAUU | Chr2: 60494602-60494624 | 194 |
| CR00256 | 58 | + | CCAAAUGUUCUUUCUUCAGC | Chr2: 60494617-60494639 | 195 |
| CR00257 | 58 | + | AUAAUAGUAUAUGCUUCAUA | Chr2: 60494676-60494698 | 196 |
| CR00258 | 58 | − | CGGAGCACUUACUCUGCUCU | Chr2: 60494737-60494759 | 197 |
| CR00259 | 58 | − | AGCAUUUUAGUUCACAAGCU | Chr2: 60494757-60494779 | 198 |
| CR00260 | 58 | − | UGUAACUAAUAAAUACCAGG | Chr2: 60494781-60494803 | 199 |
| CR00261 | 58 | − | AGGUGUAACUAAUAAAUACC | Chr2: 60494784-60494806 | 200 |
| CR00264 | 58 | − | UCUGACCCAAACUAGGAAUU | Chr2: 60494836-60494858 | 201 |
| CR00266 | 58 | + | AAGGAAAAGAAUAUGACGUC | Chr2: 60494883-60494905 | 202 |
| CR00267 | 58 | + | AGGAAAAGAAUAUGACGUCA | Chr2: 60494884-60494906 | 203 |
| CR00268 | 58 | + | GGAAAAGAAUAUGACGUCAG | Chr2: 60494885-60494907 | 204 |
| CR00269 | 58 | + | GAAAAGAAUAUGACGUCAGG | Chr2: 60494886-60494908 | 205 |
| CR00270 | 58 | + | UCAGGGGGAGGCAAGUCAGU | Chr2: 60494901-60494923 | 206 |
| CR00271 | 58 | + | CAGGGGGAGGCAAGUCAGUU | Chr2: 60494902-60494924 | 207 |
| CR00272 | 58 | | AUCACAUAUAGGCACCUAUC | Chr2: 60494939-60494961 | 208 |
| CR00273 | 58 | | CAGGUACCAGCUACUGUGUU | Chr2: 60494939-60494961 | 209 |
| CR00274 | 58 | | ACUAUCCACGGAUAUACACU | Chr2: 60494939-60494961 | 210 |
| CR00275 | 58 | + | CACAGUAGCUGGUACCUGAU | Chr2: 60494939-60494961 | 211 |
| CR00276 | 58 | − | AUCACAUAUAGGCACCUAUC | Chr2: 60494953-60494975 | 212 |
| CR00277 | 58 | + | UGAUAGGUGCCUAUAUGUGA | Chr2: 60494955-60494977 | 213 |
| CR00278 | 58 | + | AGGUGCCUAUAUGUGAUGGA | Chr2: 60494959-60494981 | 214 |
| CR00279 | 58 | + | GGUGCCUAUAUGUGAUGGAU | Chr2: 60494960-60494982 | 215 |
| CR00280 | 58 | − | UCCACCCAUCCAUCACAUAU | Chr2: 60494964-60494986 | 216 |
| CR00281 | 58 | + | ACAGCCCGACAGAUGAAAAA | Chr2: 60494986-60495008 | 217 |
| CR00282 | 58 | + | AUGAAAAAUGGACAAUUAUG | Chr2: 60494998-60495020 | 218 |
| CR00283 | 58 | + | AAAAAUGGACAAUUAUGAGG | Chr2: 60495001-60495023 | 219 |
| CR00284 | 58 | + | AAAAUGGACAAUUAUGAGGA | Chr2: 60495002-60495024 | 220 |
| CR00285 | 58 | + | AAAUGGACAAUUAUGAGGAG | Chr2: 60495003-60495025 | 221 |
| CR00286 | 58 | + | GAGGAGGGGAGAGUGCAGAC | Chr2: 60495017-60495039 | 222 |
| CR00287 | 58 | + | AGGAGGGGAGAGUGCAGACA | Chr2: 60495018-60495040 | 223 |
| CR00288 | 58 | + | CUUCACCUCCUUUACAAUUU | Chr2: 60495045-60495067 | 224 |
| CR00289 | 58 | + | UUCACCUCCUUUACAAUUUU | Chr2: 60495046-60495068 | 225 |
| CR00290 | 58 | − | GACUCCCAAAAUUGUAAAGG | Chr2: 60495050-60495072 | 226 |
| CR00291 | 58 | − | GUGGACUCCCAAAAUUGUAA | Chr2: 60495053-60495075 | 227 |
| CR00292 | 58 | + | UUUUGGGAGUCCACACGGCA | Chr2: 60495062-60495084 | 228 |
| CR00293 | 58 | − | AAUUUGUAUGCCAUGCCGUG | Chr2: 60495072-60495094 | 229 |
| CR00294 | 58 | + | CCAAGAGAGCCUUCCGAAAG | Chr2: 60495135-60495157 | 230 |
| CR00295 | 58 | − | CCAGGGGGGCCUCUUUCGGA | Chr2: 60495144-60495166 | 231 |
| CR00296 | 58 | + | CCUUCCGAAAGAGGCCCCCC | Chr2: 60495144-60495166 | 232 |
| CR00297 | 58 | + | CUUCCGAAAGAGGCCCCCCU | Chr2: 60495145-60495167 | 233 |
| CR00298 | 58 | + | AAGAGGCCCCCCUGGGCAAA | Chr2: 60495152-60495174 | 234 |
| CR00299 | 58 | − | CGGUGGCCGUUUGCCCAGGG | Chr2: 60495158-60495180 | 235 |
| CR00300 | 58 | − | UCGGUGGCCGUUUGCCCAGG | Chr2: 60495159-60495181 | 236 |
| CR00301 | 58 | − | AUCGGUGGCCGUUUGCCCAG | Chr2: 60495160-60495182 | 237 |
| CR00302 | 58 | − | CAUCGGUGGCCGUUUGCCCA | Chr2: 60495161-60495183 | 238 |
| CR00303 | 58 | + | CCUGGGCAAACGGCCACCGA | Chr2: 60495162-60495184 | 239 |
| CR00304 | 58 | − | CCAUCGGUGGCCGUUUGCCC | Chr2: 60495162-60495184 | 240 |
| CR00305 | 58 | + | GCAAACGGCCACCGAUGGAG | Chr2: 60495167-60495189 | 241 |
| CR00306 | 58 | − | CUGGCAGACCUCUCCAUCGG | Chr2: 60495175-60495197 | 242 |
| CR00307 | 58 | − | GGACUGGCAGACCUCUCCAU | Chr2: 60495178-60495200 | 243 |
| CR00308 | 58 | − | UCUGAUUAGGGUGGGGGCGU | Chr2: 60495213-60495235 | 244 |
| CR00309 | 58 | + | CACGCCCCCACCCUAAUCAG | Chr2: 60495215-60495237 | 245 |
| CR00310 | 58 | − | UUGGCCUCUGAUUAGGGUGG | Chr2: 60495219-60495241 | 246 |
| CR00311 | 58 | − | UUUGGCCUCUGAUUAGGGUG | Chr2: 60495220-60495242 | 247 |
| CR00312 | 58 | − | GUUUGGCCUCUGAUUAGGGU | Chr2: 60495221-60495243 | 248 |
| CR00313 | 58 | − | GGUUUGGCCUCUGAUUAGGG | Chr2: 60495222-60495244 | 249 |
| CR00314 | 58 | − | AAGGGUUUGGCCUCUGAUUA | Chr2: 60495225-60495247 | 250 |
| CR00315 | 58 | − | GAAGGGUUUGGCCUCUGAUU | Chr2: 60495226-60495248 | 251 |
| CR00316 | 58 | − | UUGCUUUUAUCACAGGCUCC | Chr2: 60495248-60495270 | 252 |
| CR00317 | 58 | − | CUAACAGUUGCUUUUAUCAC | Chr2: 60495255-60495277 | 253 |
| CR00318 | 58 | + | CUUCAAAGUUGUAUUGACCC | Chr2: 60495293-60495315 | 254 |
| CR00319 | 58 | − | ACUCUUAGACAUAACACACC | Chr2: 60495311-60495333 | 255 |
| CR00320 | 58 | + | UAGAUGCCAUAUCUCUUUUC | Chr2: 60495333-60495355 | 256 |
| CR00321 | 58 | − | CAUAGGCCAGAAAAGAGAUA | Chr2: 60495339-60495361 | 257 |
| CR00322 | 58 | + | GGCCUAUGUUAUUACCUGUA | Chr2: 60495354-60495376 | 258 |
| CR00323 | 58 | − | GUCCAUACAGGUAAUAACAU | Chr2: 60495356-60495378 | 259 |
| CR00324 | 58 | + | UACCUGUAUGGACUUUGCAC | Chr2: 60495366-60495388 | 260 |
| CR00325 | 58 | − | UUCCAGUGCAAAGUCCAUAC | Chr2: 60495368-60495390 | 261 |
| CR00326 | 58 | + | UGCUCUUACUUAUGCACACC | Chr2: 60495400-60495422 | 262 |
| CR00327 | 58 | + | GCUCUUACUUAUGCACACCU | Chr2: 60495401-60495423 | 263 |
| CR00328 | 58 | + | CUCUUACUUAUGCACACCUG | Chr2: 60495402-60495424 | 264 |
| CR00329 | 58 | − | CAGGGCUGGCUCUAUGCCCC | Chr2: 60495418-60495440 | 265 |
| CR00330 | 58 | − | GCUGAAAAGCGAUACAGGGC | Chr2: 60495432-60495454 | 266 |
| CR00331 | 58 | − | GAUGGCUGAAAAGCGAUACA | Chr2: 60495436-60495458 | 267 |
| CR00332 | 58 | − | AGAUGGCUGAAAAGCGAUAC | Chr2: 60495437-60495459 | 268 |
| CR00333 | 58 | − | GGGAGUUAUCUGUAGUGAGA | Chr2: 60495454-60495476 | 269 |
| CR00334 | 58 | − | AAGGCAGCUAGACAGGACUU | Chr2: 60495474-60495496 | 270 |
| CR00335 | 58 | − | GAAGGCAGCUAGACAGGACU | Chr2: 60495475-60495497 | 271 |
| CR00336 | 58 | − | GAUAAGGAAGGCAGCUAGAC | Chr2: 60495481-60495503 | 272 |
| CR00337 | 58 | + | CUAGCUGCCUUCCUUAUCAC | Chr2: 60495486-60495508 | 273 |
| CR00338 | 58 | − | UGGGUGCUAUUCCUGUGAUA | Chr2: 60495497-60495519 | 274 |
| CR00339 | 58 | + | UAUCACAGGAAUAGCACCCA | Chr2: 60495500-60495522 | 275 |
| CR00340 | 58 | − | CUGAGGUACUGAUGGACCUU | Chr2: 60495516-60495538 | 276 |
| CR00341 | 58 | − | UCUGAGGUACUGAUGGACCU | Chr2: 60495517-60495539 | 277 |
| CR001124 | 58 | − | UUAGGGUGGGGGCGUGGGUG | Chr2: 60495214-60495236 | 334 |
| CR001125 | 58 | − | UUUUAUCACAGGCUCCAGGA | Chr2: 60495215-60495237 | 335 |
| CR001126 | 58 | − | UUUAUCACAGGCUCCAGGAA | Chr2: 60495216-60495238 | 336 |
| CR001127 | 58 | − | CACAGGCUCCAGGAAGGGUU | Chr2: 60495220-60495242 | 337 |
| CR001128 | 58 | + | AUCAGAGGCCAAACCCUUCC | Chr2: 60495236-60495258 | 338 |
| CR001129 | 58 | − | CUCUGAUUAGGGUGGGGGCG | Chr2: 60495244-60495266 | 339 |
| CR001130 | 58 | − | GAUUAGGGUGGGGGCGUGGG | Chr2: 60495249-60495271 | 340 |
| CR001131 | 58 | − | AUUAGGGUGGGGGCGUGGGU | Chr2: 60495250-60495272 | 341 |

TABLE 2
Preferred Guide RNA Targeting Domains directed to the French HPFH
(French HPFH; Sankaran V G et al. A functional element necessary
for fetal hemoglobin silencing. NEJM (2011) 365: 807-814.)

| | Target | | | | SEQ |
|---|---|---|---|---|---|
| Id. | Name | Strand | gRNA Targeting Domain | Genomic Target Location | ID NO: |
| CR001016 | HPFH | − | UCUUAAACCAACCUGCUCAC | chr11: 5234538-5234558 | 86 |
| CR001017 | HPFH | + | CAGGUUGGUUUAAGAUAAGC | chr11: 5234543-5234563 | 87 |
| CR001018 | HPFH | + | AGGUUGGUUUAAGAUAAGCA | chr11: 5234544-5234564 | 88 |
| CR001019 | HPFH | − | UUAAGGGAAUAGUGGAAUGA | chr11: 5234600-5234620 | 89 |
| CR001020 | HPFH | − | AGGGCAAGUUAAGGGAAUAG | chr11: 5234608-5234628 | 90 |
| CR001021 | HPFH | + | CCCUUAACUUGCCCUGAGAU | chr11: 5234613-5234633 | 91 |
| CR001022 | HPFH | − | CCAAUCUCAGGGCAAGUUAA | chr11: 5234616-5234636 | 92 |
| CR001023 | HPFH | − | GCCAAUCUCAGGGCAAGUUA | chr11: 5234617-5234637 | 93 |
| CR001024 | HPFH | − | UGACAGAACAGCCAAUCUCA | chr11: 5234627-5234647 | 94 |
| CR001025 | HPFH | − | AUGACAGAACAGCCAAUCUC | chr11: 5234628-5234648 | 95 |
| CR001026 | HPFH | − | GAGAUAUGUAGAGGAGAACA | chr11: 5234670-5234690 | 96 |
| CR001027 | HPFH | − | GGAGAUAUGUAGAGGAGAAC | chr11: 5234671-5234691 | 97 |
| CR001028 | HPFH | − | UGCGGUGGGGAGAUAUGUAG | chr11: 5234679-5234699 | 98 |
| CR001029 | HPFH | − | CUGCUGAAAGAGAUGCGGUG | chr11: 5234692-5234712 | 99 |
| CR001030 | HPFH | − | ACUGCUGAAAGAGAUGCGGU | chr11: 5234693-5234713 | 100 |
| CR001031 | HPFH | − | AACUGCUGAAAGAGAUGCGG | chr11: 5234694-5234714 | 101 |
| CR001032 | HPFH | − | AACAACUGCUGAAAGAGAUG | chr11: 5234697-5234717 | 102 |
| CR001033 | HPFH | − | UCUGCAAAAAUGAAACUAGG | chr11: 5234731-5234751 | 103 |
| CR001034 | HPFH | − | ACUUCUGCAAAAAUGAAACU | chr11: 5234734-5234754 | 104 |
| CR001035 | HPFH | + | CAUUUUUGCAGAAGUGUUUU | chr11: 5234739-5234759 | 105 |
| CR001036 | HPFH | + | AGUGUUUUAGGCUAAUAUAG | chr11: 5234751-5234771 | 106 |
| CR001037 | HPFH | − | UUGGAGACAAAAAUCUCUAG | chr11: 5234883-5234903 | 107 |
| CR001038 | HPFH | + | UCUAGAGAUUUUUGUCUCCA | chr11: 5234882-5234902 | 108 |
| CR001039 | HPFH | + | CUAGAGAUUUUUGUCUCCAA | chr11: 5234883-5234903 | 109 |
| CR001040 | HPFH | + | GUCUCCAAGGGAAUUUUGAG | chr11: 5234895-5234915 | 110 |
| CR001041 | HPFH | + | CCAAGGGAAUUUUGAGAGGU | chr11: 5234899-5234919 | 111 |
| CR001042 | HPFH | − | CCAACCUCUCAAAAUUCCCU | chr11: 5234902-5234922 | 112 |
| CR001043 | HPFH | + | GGAAUUUUGAGAGGUUGGAA | chr11: 5234904-5234924 | 113 |
| CR001044 | HPFH | + | UGCUUGCUUCCUCCUUCUUU | chr11: 5234953-5234973 | 114 |
| CR001045 | HPFH | − | AAGAAUUUACCAAAAGAAGG | chr11: 5234965-5234985 | 115 |
| CR001046 | HPFH | − | AGGAAGAAUUUACCAAAAGA | chr11: 5234968-5234988 | 116 |
| CR001047 | HPFH | − | AAAAAUUAGAGUUUUAUUAU | chr11: 5234988-5235008 | 117 |
| CR001048 | HPFH | − | UUUUUUAAAUAUUCUUUUAA | Chr11: 5235023-5235045 | 118 |
| CR001049 | HPFH | + | UAUUUACCAGUUAUUGAAAU | chr11: 5235062-5235082 | 119 |
| CR001050 | HPFH | + | CCAGUUAUUGAAAUAGGUUC | chr11: 5235068-5235088 | 120 |
| CR001051 | HPFH | − | CCAGAACCUAUUUCAAUAAC | chr11: 5235071-5235091 | 121 |
| CR001052 | HPFH | + | UUCUGGAAACAUGAAUUUUA | chr11: 5235085-5235105 | 122 |
| CR001053 | HPFH | + | AUUUUGAAUGUUUAAAAUUA | chr11: 5235151-5235171 | 123 |
| CR001054 | HPFH | − | AAAUUUAAUCUGGCUGAAUA | chr11: 5235216-5235236 | 124 |
| CR001055 | HPFH | − | GAACUUCGUUAAAUUUAAUC | chr11: 5235226-5235246 | 125 |
| CR001056 | HPFH | + | AUUAAAUUUAACGAAGUUCC | chr11: 5235227-5235247 | 126 |
| CR001057 | HPFH | + | UUAAAUUUAACGAAGUUCCU | chr11: 5235228-5235248 | 127 |
| CR001058 | HPFH | − | UUCUGUACUAGCAUAUUCCC | chr11: 5235248-5235268 | 128 |
| CR001059 | HPFH | + | UGUGUUCUUAAAAAAAAAUG | Chr11: 5235275-5235297 | 129 |
| CR001060 | HPFH | + | AAAAAUGUGGAAUUAGACCC | chr11: 5235293-5235313 | 130 |
| CR001061 | HPFH | − | CUACUGGGAUCUUCAUUCCU | chr11: 5235313-5235333 | 131 |
| CR001062 | HPFH | − | ACUACUGGGAUCUUCAUUCC | chr11: 5235314-5235334 | 132 |
| CR001063 | HPFH | − | GAAAAGAGUGAAAAACUACU | chr11: 5235328-5235348 | 133 |
| CR001064 | HPFH | − | AGAAAAGAGUGAAAAACUAC | chr11: 5235329-5235349 | 134 |
| CR001065 | HPFH | + | GAAUUCAAAUAAUGCCACAA | chr11: 5235349-5235369 | 135 |
| CR001066 | HPFH | − | UGUGUAUUUGUCUGCCAUUG | chr11: 5235366-5235386 | 136 |
| CR001067 | HPFH | + | CACCCAUGAGCAUAUCCAAA | chr11: 5235384-5235404 | 137 |
| CR001068 | HPFH | − | UUCCUUUUGGAUAUGCUCAU | chr11: 5235389-5235409 | 138 |
| CR001069 | HPFH | − | CUUCCUUUUGGAUAUGCUCA | chr11: 5235390-5235410 | 139 |
| CR001070 | HPFH | + | CAUGAGCAUAUCCAAAAGGA | chr11: 5235388-5235408 | 140 |
| CR001071 | HPFH | + | UAUCCAAAAGGAAGGAUUGA | chr11: 5235396-5235416 | 141 |
| CR001072 | HPFH | − | UUUCCUUCAAUCCUUCCUUU | chr11: 5235402-5235422 | 142 |
| CR001073 | HPFH | + | AAGGAAGGAUUGAAGGAAAG | chr11: 5235403-5235423 | 143 |
| CR001074 | HPFH | + | GAAGGAUUGAAGGAAAGAGG | chr11: 5235406-5235426 | 144 |
| CR001075 | HPFH | + | GAGGAGGAAGAAAUGGAGAA | chr11: 5235422-5235442 | 145 |
| CR001076 | HPFH | + | AGGAAGAAAUGGAGAAAGGA | chr11: 5235426-5235446 | 146 |
| CR001077 | HPFH | + | GAAGGAAGAGGGGAAGAGAG | chr11: 5235448-5235468 | 147 |
| CR001078 | HPFH | + | GAAGAGGGGAAGAGAGAGGA | chr11: 5235452-5235472 | 148 |
| CR001079 | HPFH | + | AGGGGAAGAGAGAGGAUGGA | chr11: 5235456-5235476 | 149 |
| CR001080 | HPFH | + | GGGGAAGAGAGAGGAUGGAA | chr11: 5235457-5235477 | 150 |
| CR001081 | HPFH | + | AAGAGAGAGGAUGGAAGGGA | chr11: 5235461-5235481 | 151 |
| CR001082 | HPFH | + | AGAGAGGAUGGAAGGGAUGG | chr11: 5235464-5235484 | 152 |
| CR001083 | HPFH | + | GGAAGGGAUGGAGGAGAAGA | chr11: 5235473-5235493 | 153 |
| CR001084 | HPFH | + | GAAGAAGGAAAAAUAAAUAA | Chr11: 5235483-5235505 | 154 |
| CR001085 | HPFH | + | AGGAAAAAUAAAUAAUGGAG | Chr11: 5235488-5235510 | 155 |
| CR001086 | HPFH | + | AAAUAAAUAAUGGAGAGGAG | chr11: 5235498-5235518 | 156 |
| CR001087 | HPFH | + | UGGAGAGGAGAGGAGAAAAA | chr11: 5235508-5235528 | 157 |
| CR001088 | HPFH | + | AGAGGAGAGGAGAAAAAAGG | chr11: 5235511-5235531 | 158 |
| CR001089 | HPFH | + | GAGGAGAGGAGAAAAAAGGA | chr11: 5235512-5235532 | 159 |
| CR001090 | HPFH | + | AGGAGAGGAGAAAAAAGGAG | chr11: 5235513-5235533 | 160 |
| CR001091 | HPFH | + | AGGAGAAAAAAGGAGGGGAG | chr11: 5235518-5235538 | 161 |
| CR001092 | HPFH | + | GAGAGGAGAGGAGAAGGGAU | chr11: 5235535-5235555 | 162 |
| CR001093 | HPFH | + | AGAGGAGAGGAGAAGGGAUA | chr11: 5235536-5235556 | 163 |
| CR001094 | HPFH | + | GAAGAGAAAGAGAAAGGGAA | Chr11: 5235553-5235575 | 164 |
| CR001095 | HPFH | + | AAGAGAGGAAAGAAGAGAAG | chr11: 5235581-5235601 | 165 |
| CR001096 | HPFH | + | GAGAGAAAAGAAACGAAGAG | Chr11: 5235598-5235620 | 166 |
| CR001097 | HPFH | + | AGAGAAAAGAAACGAAGAGA | Chr11: 5235599-5235621 | 167 |
| CR001098 | HPFH | + | GAGAAAAGAAACGAAGAGAG | Chr11: 5235600-5235622 | 168 |
| CR001099 | HPFH | + | AAAGAAACGAAGAGAGGGGA | chr11: 5235609-5235629 | 169 |
| CR001100 | HPFH | + | AAGAAACGAAGAGAGGGGAA | chr11: 5235610-5235630 | 170 |
| CR001101 | HPFH | + | GGAAGGGAAGGAAAAAAAAG | chr11: 5235626-5235646 | 171 |
| CR001102 | HPFH | + | AAGACUGACAGUUCAAAUUU | chr11: 5235672-5235692 | 172 |
| CR001103 | HPFH | + | ACUGACAGUUCAAAUUUUGG | chr11: 5235675-5235695 | 173 |
| CR001104 | HPFH | + | UUCAAAUUUUGGUGGUGAUA | chr11: 5235683-5235703 | 174 |
| CR001105 | HPFH | + | AAUAGAAACUCAAACUCUGU | chr11: 5235709-5235729 | 175 |
| CR001106 | HPFH | + | GUACAAUAGUAUAACCCCUU | chr11: 5235739-5235759 | 176 |
| CR001107 | HPFH | − | CUAUUAAAGGUUUUCCAAAG | chr11: 5235756-5235776 | 177 |
| CR001108 | HPFH | − | ACUAUUAAAGGUUUUCCAAA | chr11: 5235757-5235777 | 178 |
| CR001109 | HPFH | − | UACUAUUAAAGGUUUUCCAA | chr11: 5235758-5235778 | 179 |
| CR001110 | HPFH | − | GCAUUUGUGGAUACUAUUAA | chr11: 5235769-5235789 | 180 |
| CR001111 | HPFH | − | UUAAUAGUAUCCACAAAUGC | chr11: 5235769-5235789 | 181 |
| CR001132 | HPFH | − | UAUCAAGCAUCCAGCAUUUG | chr11: 5235782-5235802 | 342 |
| CR001133 | HPFH | − | UAUCUAAAAAUGUAAUUGCU | chr11: 5235814-5235834 | 343 |
| CR001134 | HPFH | − | AGCAUUUCUAUACAUGUCUU | chr11: 5235862-5235882 | 344 |
| CR001135 | HPFH | − | UAAUCAUAAAAACCUCAAAC | chr11: 5235893-5235913 | 345 |
| CR001136 | HPFH | − | UUUAAGUGGCUACCGGUUUG | chr11: 5235908-5235928 | 346 |
| CR001137 | HPFH | − | GUAAGCAUUUAAGUGGCUAC | chr11: 5235915-5235935 | 347 |
| CR001138 | HPFH | − | ACUGUUGGUAAGCAUUUAAG | chr11: 5235922-5235942 | 348 |
| CR001139 | HPFH | − | UAAUUUAUCAAUUCUACUGU | chr11: 5235937-5235957 | 349 |
| CR001140 | HPFH | + | ACAGUAGAAUUGAUAAAUUA | chr11: 5235937-5235957 | 350 |
| CR001141 | HPFH | + | CAAAUGCAUUUUACAGCAUU | chr11: 5236027-5236047 | 351 |
| CR001142 | HPFH | + | GGUUGAUUAAAAGUAACCAG | chr11: 5236048-5236068 | 352 |
| CR001143 | HPFH | − | AUAUAGUUUGAACUCACCUC | Chr11: 5236059-5236081 | 353 |
| CR001144 | HPFH | + | UUUAUUUGUAUAUAGAAAGA | chr11: 5236090-5236110 | 354 |
| CR001145 | HPFH | + | UGCCUGAGAUUCUGAUCACA | chr11: 5236119-5236139 | 355 |
| CR001146 | HPFH | + | GCCUGAGAUUCUGAUCACAA | chr11: 5236120-5236140 | 356 |
| CR001147 | HPFH | + | CCUGAGAUUCUGAUCACAAG | chr11: 5236121-5236141 | 357 |
| CR001148 | HPFH | − | CCCCUUGUGAUCAGAAUCUC | chr11: 5236124-5236144 | 358 |
| CR001149 | HPFH | + | AAGGGGAAAUGUUAUAAAAU | chr11: 5236138-5236158 | 359 |
| CR001150 | HPFH | + | AGGGGAAAUGUUAUAAAAUA | chr11: 5236139-5236159 | 360 |
| CR001151 | HPFH | + | UGUUAUAAAAUAGGGUAGAG | chr11: 5236147-5236167 | 361 |
| CR001152 | HPFH | − | CAAAGUUUAAAGGUCAUUCA | chr11: 5236175-5236195 | 362 |
| CR001153 | HPFH | − | UAACUUGUAACAAAGUUUAA | chr11: 5236185-5236205 | 363 |
| CR001154 | HPFH | + | CAAGUUAUUUUUCUGUAACC | chr11: 5236198-5236218 | 364 |
| CR001155 | HPFH | − | AAUAUCUUUCGUUGGCUUCC | chr11: 5236219-5236239 | 365 |
| CR001156 | HPFH | − | AAUUAUUCAAUAUCUUUCGU | chr11: 5236227-5236247 | 366 |
| CR001157 | HPFH | + | GAUAUUGAAUAAUUCAAGAA | chr11: 5236233-5236253 | 367 |
| CR001158 | HPFH | + | AUUGAAUAAUUCAAGAAAGG | chr11: 5236236-5236256 | 368 |
| CR001159 | HPFH | + | GAAUAAUUCAAGAAAGGUGG | chr11: 5236239-5236259 | 369 |
| CR001160 | HPFH | + | AUUCAAGAAAGGUGGUGGCA | chr11: 5236244-5236264 | 370 |
| CR001161 | HPFH | + | UAUUUUAGAAGUAGAGAAAA | chr11: 5236313-5236333 | 371 |
| CR001162 | HPFH | + | AUUUUAGAAGUAGAGAAAAU | chr11: 5236314-5236334 | 372 |
| CR001163 | HPFH | + | GAAAAUGGGAGACAAAUAGC | chr11: 5236328-5236348 | 373 |
| CR001164 | HPFH | + | AAAAUGGGAGACAAAUAGCU | chr11: 5236329-5236349 | 374 |
| CR001165 | HPFH | + | AGCUGGGCUUCUGUUGCAGU | chr11: 5236345-5236365 | 375 |
| CR001166 | HPFH | + | GCUGGGCUUCUGUUGCAGUA | chr11: 5236346-5236366 | 376 |
| CR001167 | HPFH | + | GCCAUUUCUAUUAUCAGACU | chr11: 5236383-5236403 | 377 |
| CR001168 | HPFH | − | UCCAAGUCUGAUAAUAGAAA | chr11: 5236387-5236407 | 378 |
| CR001169 | HPFH | + | UUAUCAGACUUGGACCAUGA | chr11: 5236393-5236413 | 379 |
| CR001170 | HPFH | − | CACGACUGACAUCACCGUCA | chr11: 5236410-5236430 | 380 |
| CR001171 | HPFH | + | UCAGUCGUGAACACAAGAAU | chr11: 5236421-5236441 | 381 |
| CR001172 | HPFH | + | CAGUCGUGAACACAAGAAUA | chr11: 5236422-5236442 | 382 |
| CR001173 | HPFH | + | GGCCACAUUUGUGAGUUUAG | chr11: 5236443-5236463 | 383 |
| CR001174 | HPFH | − | UACCACUAAACUCACAAAUG | chr11: 5236448-5236468 | 384 |
| CR001175 | HPFH | + | UAAAAUCAGAAAUACAGUCU | chr11: 5236471-5236491 | 385 |
| CR001176 | HPFH | + | AAAAGAUGUACUUAGAUAUG | chr11: 5236528-5236548 | 386 |
| CR001177 | HPFH | + | UGUACUUAGAUAUGUGGAUC | chr11: 5236534-5236554 | 387 |
| CR001178 | HPFH | + | AGCUCAGAAAGAAUACAACC | chr11: 5236557-5236577 | 388 |
| CR001179 | HPFH | + | ACCAGGUCAAGAAUACAGAA | chr11: 5236574-5236594 | 389 |
| CR001180 | HPFH | − | UCCAUUCUGUAUUCUUGACC | chr11: 5236578-5236598 | 390 |
| CR001181 | HPFH | − | CUGUCAUUUUUAACAGGUAG | chr11: 5236646-5236666 | 391 |
| CR001182 | HPFH | − | CAUCAUCUGUCAUUUUUAAC | chr11: 5236652-5236672 | 392 |
| CR001183 | HPFH | − | AAACACAUUCUAAGAUUUUA | chr11: 5236691-5236711 | 393 |
| CR001184 | HPFH | + | AAUCUUAGAAUGUGUUUGUG | chr11: 5236694-5236714 | 394 |
| CR001185 | HPFH | + | AUCUUAGAAUGUGUUUGUGA | chr11: 5236695-5236715 | 395 |
| CR001186 | HPFH | + | UUAGAAUGUGUUUGUGAGGG | chr11: 5236698-5236718 | 396 |
| CR001187 | HPFH | − | CAAUUUUCUUAUAUAUGAAU | chr11: 5236734-5236754 | 397 |
| CR001188 | HPFH | + | UUGAUUCUAAAAAAAAUGUU | Chr11: 5236746-5236768 | 398 |
| CR001189 | HPFH | + | AAAUGUUAGGUAAAUUCUUA | chr11: 5236764-5236784 | 399 |
| CR001190 | HPFH | + | GGUAAAUUCUUAAGGCCAUG | chr11: 5236772-5236792 | 400 |
| CR001191 | HPFH | − | AGAUCAAAUAACAGUCCUCA | chr11: 5236790-5236810 | 401 |
| CR001192 | HPFH | + | GUCUGUUAAUUCCAAAGACU | chr11: 5236812-5236832 | 402 |
| CR001193 | HPFH | − | AAAGUGAAAAGCCAAGUCUU | chr11: 5236826-5236846 | 403 |
| CR001194 | HPFH | + | CCUGAAAUGAUUUUACACAU | chr11: 5236858-5236878 | 404 |
| CR001195 | HPFH | − | CCAAUGUGUAAAAUCAUUUC | chr11: 5236861-5236881 | 405 |
| CR001196 | HPFH | + | CUGAAAUGAUUUUACACAUU | chr11: 5236859-5236879 | 406 |
| CR001197 | HPFH | + | AUUUUACACAUUGGGAGAUC | chr11: 5236867-5236887 | 407 |
| CR001198 | HPFH | + | GGUUACAUGUUUAUUCUAUA | chr11: 5236888-5236908 | 408 |
| CR001199 | HPFH | + | UCUAUAUGGAUUGCAUUGAG | chr11: 5236902-5236922 | 409 |
| CR001200 | HPFH | + | AGGAUUUGUAUAACAGAAUA | chr11: 5236922-5236942 | 410 |
| CR001201 | HPFH | + | UUUUCUUUUCUCUUCUGAGA | Chr11: 5236945-5236967 | 411 |
| CR001202 | HPFH | − | GCACUCUAGCUUGGGCAAUA | chr11: 5236984-5237004 | 412 |
| CR001203 | HPFH | − | UGCACUCUAGCUUGGGCAAU | chr11: 5236985-5237005 | 413 |
| CR001204 | HPFH | − | UGCACCAUUGCACUCUAGCU | Chr11: 5236985-5237007 | 414 |
| CR001205 | HPFH | − | GCUAUUCAGGUGGCUGAGGC | chr11: 5237061-5237081 | 415 |
| CR001206 | HPFH | + | ACCUGAAUAGCUGGGACUGC | Chr11: 5237065-5237087 | 416 |
| CR001207 | HPFH | + | GCAGGCAUGCACCACACGCC | Chr11: 5237083-5237105 | 417 |
| CR001208 | HPFH | − | UACAAAAUCAGCCGGGCGUG | chr11: 5237102-5237122 | 418 |
| CR001209 | HPFH | − | GGCUUGUAAACCCAGCACUU | chr11: 5237208-5237228 | 419 |
| CR001210 | HPFH | − | CUGGCUGGAUGCGGUGGCUC | chr11: 5237229-5237249 | 420 |
| CR001211 | HPFH | + | CUGAGCCACCGCAUCCAGCC | chr11: 5237227-5237247 | 421 |
| CR001212 | HPFH | − | CUUAUCCUGGCUGGAUGCGG | chr11: 5237235-5237255 | 422 |
| CR001213 | HPFH | + | CACCGCAUCCAGCCAGGAUA | chr11: 5237233-5237253 | 423 |
| CR001214 | HPFH | − | GACCUUAUCCUGGCUGGAUG | chr11: 5237238-5237258 | 424 |
| CR001215 | HPFH | − | CUUUUAGACCUUAUCCUGGC | chr11: 5237244-5237264 | 425 |
| CR001216 | HPFH | + | GCCAGGAUAAGGUCUAAAAG | chr11: 5237244-5237264 | 426 |
| CR001217 | HPFH | − | UCCACUUUUAGACCUUAUCC | chr11: 5237248-5237268 | 427 |
| CR001218 | HPFH | + | AAUAGCAUCUACUCUUGUUC | chr11: 5237271-5237291 | 428 |
| CR001219 | HPFH | + | CUCUUGUUCAGGAAACAAUG | chr11: 5237282-5237302 | 429 |
| CR001220 | HPFH | + | GGAAACAAUGAGGACCUGAC | chr11: 5237292-5237312 | 430 |
| CR001221 | HPFH | + | GAAACAAUGAGGACCUGACU | chr11: 5237293-5237313 | 431 |
| CR001222 | HPFH | + | ACCUGACUGGGCAGUAAGAG | chr11: 5237305-5237325 | 432 |
| CR001223 | HPFH | − | ACCACUCUUACUGCCCAGUC | chr11: 5237309-5237329 | 433 |
| CR001224 | HPFH | + | AAGAGUGGUGAUUAAUAGAU | chr11: 5237320-5237340 | 434 |
| CR001225 | HPFH | + | AGAGUGGUGAUUAAUAGAUA | chr11: 5237321-5237341 | 435 |
| CR001226 | HPFH | + | AGAAUCGAACUGUUGAUUAG | chr11: 5237356-5237376 | 436 |
| CR001227 | HPFH | + | UCGAACUGUUGAUUAGAGGU | chr11: 5237360-5237380 | 437 |
| CR003027 | HPFH | + | CGAACUGUUGAUUAGAGGUA | chr11: 5237361-5237381 | 438 |
| CR003028 | HPFH | + | AUGAUUUUAAUCUGUGACCU | chr11: 5237386-5237406 | 439 |
| CR003029 | HPFH | + | UAAUCUGUGACCUUGGUGAA | chr11: 5237393-5237413 | 440 |
| CR003030 | HPFH | + | AAUCUGUGACCUUGGUGAAU | chr11: 5237394-5237414 | 441 |
| CR003031 | HPFH | − | AGCUACUUGCCCAUUCACCA | chr11: 5237406-5237426 | 442 |
| CR003032 | HPFH | + | UAGCUAUCUAAUGACUAAAA | chr11: 5237421-5237441 | 443 |
| CR003033 | HPFH | + | AUGACUAAAAUGGAAAACAC | chr11: 5237431-5237451 | 444 |
| CR003034 | HPFH | + | AAAUACCCAUGCUGAGUCUG | chr11: 5237482-5237502 | 445 |
| CR003035 | HPFH | − | AGGCACCUCAGACUCAGCAU | chr11: 5237490-5237510 | 446 |
| CR003036 | HPFH | − | UAGGCACCUCAGACUCAGCA | chr11: 5237491-5237511 | 447 |
| CR003037 | HPFH | + | GCUGAGUCUGAGGUGCCUAU | chr11: 5237492-5237512 | 448 |
| CR003038 | HPFH | − | UAUUUAUAUAGAUGUCCUAU | chr11: 5237510-5237530 | 449 |
| CR003039 | HPFH | − | CAUAUAUCAAACAAUGUACU | chr11: 5237535-5237555 | 450 |
| CR003040 | HPFH | + | CCAGUACAUUGUUUGAUAUA | chr11: 5237533-5237553 | 451 |
| CR003041 | HPFH | − | CCAUAUAUCAAACAAUGUAC | chr11: 5237536-5237556 | 452 |
| CR003042 | HPFH | + | CAGUACAUUGUUUGAUAUAU | chr11: 5237534-5237554 | 453 |
| CR003043 | HPFH | + | CAUUGUUUGAUAUAUGGGUU | chr11: 5237539-5237559 | 454 |
| CR003044 | HPFH | + | GAUAUAUGGGUUUGGCACUG | chr11: 5237547-5237567 | 455 |
| CR003045 | HPFH | + | UAUGGGUUUGGCACUGAGGU | chr11: 5237551-5237571 | 456 |
| CR003046 | HPFH | + | GGGUUUGGCACUGAGGUUGG | chr11: 5237554-5237574 | 457 |
| CR003047 | HPFH | + | GCACUGAGGUUGGAGGUCAG | chr11: 5237561-5237581 | 458 |
| CR003048 | HPFH | + | CAGAGGUUAGAAAUCAGAGU | chr11: 5237578-5237598 | 459 |
| CR003049 | HPFH | + | AGAGGUUAGAAAUCAGAGUU | chr11: 5237579-5237599 | 460 |
| CR003050 | HPFH | + | UAGAAAUCAGAGUUGGGAAU | chr11: 5237585-5237605 | 461 |
| CR003051 | HPFH | + | AGAAAUCAGAGUUGGGAAUU | chr11: 5237586-5237606 | 462 |
| CR003052 | HPFH | + | GUUGGGAAUUGGGAUUAUAC | chr11: 5237596-5237616 | 463 |
| CR003053 | HPFH | − | CUUUGUAUUCAUCACACUCU | chr11: 5237654-5237674 | 464 |
| CR003054 | HPFH | + | AUGAAUACAAAGUUAAAUGA | chr11: 5237662-5237682 | 465 |
| CR003055 | HPFH | − | UAAAUGUUGGUGUUCAUUAA | chr11: 5237689-5237709 | 466 |
| CR003056 | HPFH | − | UGAGAUUUCACAUUAAAUGU | chr11: 5237702-5237722 | 467 |
| CR003057 | HPFH | + | ACAUUUAAUGUGAAAUCUCA | chr11: 5237702-5237722 | 468 |
| CR003058 | HPFH | − | UAAAAUCAUCGGGGAUUUUG | chr11: 5237749-5237769 | 469 |
| CR003059 | HPFH | − | CUAAAAUCAUCGGGGAUUUU | chr11: 5237750-5237770 | 470 |
| CR003060 | HPFH | − | UCUAAAAUCAUCGGGGAUUU | chr11: 5237751-5237771 | 471 |
| CR003061 | HPFH | − | ACUGAGUUCUAAAAUCAUCG | chr11: 5237758-5237778 | 472 |
| CR003062 | HPFH | − | UACUGAGUUCUAAAAUCAUC | chr11: 5237759-5237779 | 473 |
| CR003063 | HPFH | − | AUACUGAGUUCUAAAAUCAU | chr11: 5237760-5237780 | 474 |
| CR003064 | HPFH | + | UAAUUAGUGUAAUGCCAAUG | chr11: 5237786-5237806 | 475 |
| CR003065 | HPFH | + | AAUUAGUGUAAUGCCAAUGU | chr11: 5237787-5237807 | 476 |
| CR003066 | HPFH | + | AAUGCCAAUGUGGGUUAGAA | chr11: 5237796-5237816 | 477 |
| CR003067 | HPFH | − | ACUUCCAUUCUAACCCACAU | chr11: 5237803-5237823 | 478 |
| CR003068 | HPFH | + | AAUGGAAGUCAACUUGCUGU | chr11: 5237814-5237834 | 479 |
| CR003069 | HPFH | + | CUUGCUGUUGGUUUCAGAGC | chr11: 5237826-5237846 | 480 |
| CR003070 | HPFH | + | CUGUUGGUUUCAGAGCAGGU | chr11: 5237830-5237850 | 481 |
| CR003071 | HPFH | + | UUCAGAGCAGGUAGGAGAUA | chr11: 5237838-5237858 | 482 |
| CR003072 | HPFH | + | AGUGAAAAGCUGAAACAAAA | chr11: 5237877-5237897 | 483 |
| CR003073 | HPFH | + | AAGCUGAAACAAAAAGGAAA | chr11: 5237883-5237903 | 484 |
| CR003074 | HPFH | + | UGAAACAAAAAGGAAAAGGU | chr11: 5237887-5237907 | 485 |
| CR003075 | HPFH | + | GAAACAAAAAGGAAAAGGUA | chr11: 5237888-5237908 | 486 |
| CR003076 | HPFH | + | GGAAAAGGUAGGGUGAAAGA | chr11: 5237898-5237918 | 487 |
| CR003077 | HPFH | + | GAAAAGGUAGGGUGAAAGAU | chr11: 5237899-5237919 | 488 |
| CR003078 | HPFH | + | AAAGAUGGGAAAUGUAUGUA | chr11: 5237913-5237933 | 489 |
| CR003079 | HPFH | + | GAUGGGAAAUGUAUGUAAGG | chr11: 5237916-5237936 | 490 |
| CR003080 | HPFH | + | UGUAAGGAGGAUGAGCCACA | chr11: 5237929-5237949 | 491 |
| CR003081 | HPFH | + | GGAGGAUGAGCCACAUGGUA | chr11: 5237934-5237954 | 492 |
| CR003082 | HPFH | + | GAGGAUGAGCCACAUGGUAU | chr11: 5237935-5237955 | 493 |
| CR003083 | HPFH | + | GAUGAGCCACAUGGUAUGGG | chr11: 5237938-5237958 | 494 |
| CR003084 | HPFH | − | AGUAUACCUCCCAUACCAUG | chr11: 5237947-5237967 | 495 |
| CR003085 | HPFH | + | AUGGUAUGGGAGGUAUACUA | chr11: 5237948-5237968 | 496 |
| CR003086 | HPFH | + | GGAGGUAUACUAAGGACUCU | chr11: 5237956-5237976 | 497 |
| CR003087 | HPFH | + | GAGGUAUACUAAGGACUCUA | chr11: 5237957-5237977 | 498 |
| CR003088 | HPFH | + | ACUCUAGGGUCAGAGAAAUA | chr11: 5237971-5237991 | 499 |
| CR003089 | HPFH | + | CUCUAGGGUCAGAGAAAUAU | chr11: 5237972-5237992 | 500 |
| CR003090 | HPFH | − | AAGAAUGUGAAUUUUGUAGA | chr11: 5238004-5238024 | 501 |
| CR003091 | HPFH | + | UUCUACAAAAUUCACAUUCU | chr11: 5238003-5238023 | 502 |
| CR003092 | HPFH | + | ACAAAAUUCACAUUCUUGGC | chr11: 5238007-5238027 | 503 |
| CR003093 | HPFH | + | CAAAAUUCACAUUCUUGGCU | chr11: 5238008-5238028 | 504 |
| CR003094 | HPFH | + | UUCACAUUCUUGGCUGGGUG | chr11: 5238013-5238033 | 505 |
| CR003095 | HPFH | + | AGGGUGGAUCACCUGAUGUU | chr11: 5238071-5238093 | 506 |
| CR003096 | HPFH | − | GAUCUCGAACUCCUAACAUC | chr11: 5238090-5238110 | 507 |

TABLE 3
gRNA targeting domains directed HBG promoter regions, including those regions
of the HBG promoters that include nondeletional HPFH regions. SEQ ID NO:s refer to the
gRNA targeting domain sequence.

| Targeting | Target | gRNA targeting | genomic | | genomic | | SEQ |
|---|---|---|---|---|---|---|---|
| Domain | Promoter | domain | location (hg38) | | location (hg38) | | ID |
| ID | Region | sequence | 1 | strand | 2 (if present) | strand | NO: |

gRNA targeting domains with target sequences only within the HBG1 promoter region

| | | | | | |
|---|---|---|---|---|---|
| GCR- | HBG1 | AGUCCUGGU | chr11:5250169- | − | 1 |
| 0001 | | AUCCUCUAU | 5250189 | | |
| | | GA | | | |
| GCR- | HBG1 | AAUUAGCAG | chr11:5250063- | − | 2 |
| 0002 | | UAUCCUCUU | 5250083 | | |
| | | GG | | | |
| GCR- | HBG1 | AGAAUAAAU | chr11:5250123- | − | 3 |
| 0003 | | UAGAGAAAA | 5250143 | | |
| | | AC | | | |
| GCR- | HBG1 | AAAAAUUAG | chr11:5250066- | − | 4 |
| 0004 | | CAGUAUCCU | 5250086 | | |
| | | CU | | | |
| GCR- | HBG1 | AAAAUUAGC | chr11:5250065- | − | 5 |
| 0005 | | AGUAUCCUC | 5250085 | | |
| | | UU | | | |
| GCR- | HBG1 | AAAAACUGG | chr11:5250109- | − | 6 |
| 0006 | | AAUGACUGA | 5250129 | | |
| | | AU | | | |
| GCR- | HBG1 | CUCCCAUCA | chr11:5250163- | + | 7 |
| 0007 | | UAGAGGAUA | 5250183 | | |
| | | CC | | | |
| GCR- | HBG1 | GGAGAAGGA | chr11:5250147- | − | 8 |
| 0008 | | AACUAGCUA | 5250167 | | |
| | | AA | | | |
| GCR- | HBG1 | GUUUCCUUC | chr11:5250155- | + | 9 |
| 0009 | | UCCCAUCAU | 5250175 | | |
| | | AG | | | |
| GCR- | HBG1 | GGGAGAAGG | chr11:5250148- | − | 10 |
| 0010 | | AAACUAGCU | 5250168 | | |
| | | AA | | | |
| GCR- | HBG1 | CACUGGAGC | chr11:5250213- | − | 11 |
| 0011 | | UAGAGACAA | 5250233 | | |
| | | GA | | | |
| GCR- | HBG1 | AGAGACAAG | chr11:5250203- | − | 12 |
| 0012 | | AAGGUAAAA | 5250223 | | |
| | | AA | | | |
| GCR- | HBG1 | AAAUUAGCA | chr11:5250064- | − | 13 |
| 0013 | | GUAUCCUCU | 5250084 | | |
| | | UG | | | |
| GCR- | HBG1 | GUCCUGGUA | chr11:5250168- | − | |
| 0014 | | UCCUCUAUG | 5250188 | | |
| | | AU | | | 14 |
| GCR- | HBG1 | GUAUCCUCU | chr11:5250162- | − | |
| 0015 | | AUGAUGGGA | 5250182 | | |
| | | GA | | | 15 |

gRNA targeting domains with target sequences only within the HBG2 promoter region

| | | | | | |
|---|---|---|---|---|---|
| GCR- | HBG2 | AUUAAGCAG | chr11:5254990- | − | 17 |
| 0017 | | CAGUAUCCU | 5255010 | | |
| | | CU | | | |
| GCR- | HBG2 | AGAAUAAAU | chr11:5255051- | − | 22 |
| 0022 | | UAGAGAAAA | 5255071 | | |
| | | AU | | | |
| GCR- | HBG2 | AGAAGUCCU | chr11:5255100- | − | 29 |
| 0029 | | GGUAUCUUC | 5255120 | | |
| | | UA | | | |
| GCR- | HBG2 | UUAAGCAGC | chr11:5254989- | − | |
| 0032 | | AGUAUCCUC | 5255009 | | |
| | | UU | | | 32 |
| GCR- | HBG2 | AAAAAUUGG | chr11:5255037- | − | 34 |
| 0034 | | AAUGACUGA | 5255057 | | |
| | | AU | | | |
| GCR- | HBG2 | GGGAGAAGA | chr11:5255076- | − | 46 |
| 0046 | | AAACUAGCU | 5255096 | | |
| | | AA | | | |
| GCR- | HBG2 | GGAGAAGAA | chr11:5255075- | − | 51 |
| 0051 | | AACUAGCUA | 5255095 | | |
| | | AA | | | |
| GCR- | HBG2 | CUCCCACCA | chr11:5255091- | + | 52 |
| 0052 | | UAGAAGAUA | 5255111 | | |
| | | CC | | | |
| GCR- | HBG2 | AGUCCUGGU | chr11:5255097- | − | 54 |
| 0054 | | AUCUUCUAU | 5255117 | | |
| | | GG | | | |
| GCR- | HBG2 | GUCCUGGUA | chr11:5255096- | − | 58 |
| 0058 | | UCUUCUAUG | 5255116 | | |
| | | GU | | | |
| GCR- | HBG2 | UAAGCAGCA | chr11:5254988- | − | 60 |
| 0060 | | GUAUCCUCU | 5255008 | | |
| | | UG | | | |
| GCR- | HBG2 | AAGCAGCAG | chr11:5254987- | − | 69 |
| 0069 | | UAUCCUCUU | 5255007 | | |
| | | GG | | | |

gRNA with targeting domains within the HBG1 and HBG2 promoter regions

| | | | | | | | |
|---|---|---|---|---|---|---|---|
| GCR- | HBG1/H | CCUAGCCAG | chr11:5249895- | + | chr11:5254819- | + | 16 |
| 0016 | BG2 | CCGCCGGCCC | 5249915 | | 5254839 | | |
| | | C | | | | | |
| GCR- | HBG1/H | UAUCCAGUG | chr11:5249910- | − | chr11:5254834- | − | 18 |
| 0018 | BG2 | AGGCCAGGG | 5249930 | | 5254854 | | |
| | | GC | | | | | |
| GCR- | HBG1/H | CAUUGAGAU | chr11:5250036- | + | chr11:5254960- | + | 19 |
| 0019 | BG2 | AGUGUGGGG | 5250056 | | 5254980 | | |
| | | AA | | | | | |
| GCR- | HBG1/H | CCAGUGAGG | chr11:5249907- | − | chr11:5254831- | − | 20 |
| 0020 | BG2 | CCAGGGGCC | 5249927 | | 5254851 | | |
| | | GG | | | | | |
| GCR- | HBG1/H | GUGGGGAAG | chr11:5250048- | + | chr11:5254972- | + | 21 |
| 0021 | BG2 | GGGCCCCCA | 5250068 | | 5254992 | | |
| | | AG | | | | | |
| GCR- | HBG1/H | CCAGGGGCC | chr11:5249898- | − | chr11:5254822- | − | 23 |
| 0023 | BG2 | GGCGGCUGG | 5249918 | | 5254842 | | |
| | | CU | | | | | |
| GCR- | HBG1/H | UGAGGCCAG | chr11:5249903- | − | chr11:5254827- | − | 24 |
| 0024 | BG2 | GGGCCGGCG | 5249923 | | 5254847 | | |
| | | GC | | | | | |
| GCR- | HBG1/H | CAGUUCCAC | chr11:5249846- | − | chr11:5254770- | − | 25 |
| 0025 | BG2 | ACACUCGCU | 5249866 | | 5254790 | | |
| | | UC | | | | | |
| GCR- | HBG1/H | CCGCCGGCCC | chr11:5249904- | + | chr11:5254828- | + | 26 |
| 0026 | BG2 | CUGGCCUCA | 5249924 | | 5254848 | | |
| | | C | | | | | |
| GCR- | HBG1/H | GUUUGCCUU | chr11:5249949- | + | chr11:5254873- | + | 27 |
| 0027 | BG2 | GUCAAGGCU | 5249969 | | 5254893 | | |
| | | AU | | | | | |
| GCR- | HBG1/H | GGCUAGGGA | chr11:5249882- | − | chr11:5254806- | − | 28 |
| 0028 | BG2 | UGAAGAAUA | 5249902 | | 5254826 | | |
| | | AA | | | | | |
| GCR- | HBG1/H | CAGGGGCCG | chr11:5249897- | − | chr11:5254821- | − | 30 |
| 0030 | BG2 | GCGGCUGGC | 5249917 | | 5254841 | | |
| | | UA | | | | | |
| GCR- | HBG1/H | ACUGGAUAC | chr11:5249922- | + | chr11:5254846- | + | 31 |
| 0031 | BG2 | UCUAAGACU | 5249942 | | 5254866 | | |
| | | AU | | | | | |
| GCR- | HBG1/H | CCCUGGCUA | chr11:5249995- | − | chr11:5254919- | − | 33 |
| 0033 | BG2 | AACUCCACC | 5250015 | | 5254939 | | |
| | | CA | | | | | |
| GCR- | HBG1/H | UUAGAGUAU | chr11:5249916- | − | chr11:5254840- | − | 35 |
| 0035 | BG2 | CCAGUGAGG | 5249936 | | 5254860 | | |
| | | CC | | | | | |
| GCR- | HBG1/H | CCCAUGGGU | chr1l:5249991- | + | chr11:5254915- | + | 36 |
| 0036 | BG2 | GGAGUUUAG | 5250011 | | 5254935 | | |
| | | CC | | | | | |
| GCR- | HBG1/H | AGGCAAGGC | chr11:5249975- | + | chr11:5254899- | + | 37 |
| 0037 | BG2 | UGGCCAACC | 5249995 | | 5254919 | | |
| | | CA | | | | | |
| GCR- | HBG1/H | UAGAGUAUC | chr1l:5249915- | − | chr11:5254839- | − | 38 |
| 0038 | BG2 | CAGUGAGGC | 5249935 | | 5254859 | | |
| | | CA | | | | | |
| GCR- | HBG1/H | UAUCUGUCU | chr11:5250012- | − | chr11:5254936- | − | 39 |
| 0039 | BG2 | GAAACGGUC | 5250032 | | 5254956 | | |
| | | CC | | | | | |
| GCR- | HBG1/H | AUUGAGAUA | chr11:5250037- | + | chr11:5254961- | + | 40 |
| 0040 | BG2 | GUGUGGGGA | 5250057 | | 5254981 | | |
| | | AG | | | | | |
| GCR- | HBG1/H | CUUCAUCCC | chr11:5249888- | + | chr11:5254812- | + | 41 |
| 0041 | BG2 | UAGCCAGCC | 5249908 | | 5254832 | | |
| | | GC | | | | | |
| GCR- | HBG1/H | GCUAUUGGU | chr11:5249964- | + | chr11:5254888- | + | 42 |
| 0042 | BG2 | CAAGGCAAG | 5249984 | | 5254908 | | |
| | | GC | | | | | |
| GCR- | HBG1/H | AUGCAAAUA | chr11:5250019- | − | chr11:5254943- | − | 43 |
| 0043 | BG2 | UCUGUCUGA | 5250039 | | 5254963 | | |
| | | AA | | | | | |
| GCR- | HBG1/H | GCAUUGAGA | chr11:5250035- | + | chr11:5254959- | + | 44 |
| 0044 | BG2 | UAGUGUGGG | 5250055 | | 5254979 | | |
| | | GA | | | | | |
| GCR- | HBG1/H | UGGUCAAGU | chr11:5249942- | + | chr11:5254866- | + | 45 |
| 0045 | BG2 | UUGCCUUGU | 5249962 | | 5254886 | | |
| | | CA | | | | | |
| GCR- | HBG1/H | GGCAAGGCU | chr11:5249976- | + | chr11:5254900- | + | 47 |
| 0047 | BG2 | GGCCAACCC | 5249996 | | 5254920 | | |
| | | AU | | | | | |
| GCR- | HBG1/H | ACGGCUGAC | chr11:5250184- | − | chr11:5255112- | − | 48 |
| 0048 | BG2 | AAAAGAAGU | 5250204 | | 5255132 | | |
| | | CC | | | | | |
| GCR- | HBG1/H | CGAGUGUGU | chr11:5249850- | + | chr11:5254774- | + | 49 |
| 0049 | BG2 | GGAACUGCU | 5249870 | | 5254794 | | |
| | | GA | | | | | |
| GCR- | HBG1/H | CCUGGCUAA | chr11:5249994- | − | chr11:5254918- | − | 50 |
| 0050 | BG2 | ACUCCACCC | 5250014 | | 5254938 | | |
| | | AU | | | | | |
| GCR- | HBG1/H | CUUGUCAAG | chr11:5249955- | + | chr11:5254879- | + | 53 |
| 0053 | BG2 | GCUAUUGGU | 5249975 | | 5254899 | | |
| | | CA | | | | | |
| GCR- | HBG1/H | AUAUUUGCA | chr11:5250029- | + | chr11:5254953- | + | 55 |
| 0055 | BG2 | UUGAGAUAG | 5250049 | | 5254973 | | |
| | | UG | | | | | |
| GCR- | HBG1/H | GCUAAACUC | chr11:5249990- | − | chr11:5254914- | − | 56 |
| 0056 | BG2 | CACCCAUGG | 5250010 | | 5254934 | | |
| | | GU | | | | | |
| GCR- | HBG1/H | ACGUUCCAG | chr11:5249838- | + | chr11:5254762- | + | 57 |
| 0057 | BG2 | AAGCGAGUG | 5249858 | | 5254782 | | |
| | | UG | | | | | |
| GCR- | HBG1/H | UAUUUGCAU | chr11:5250030- | + | chr11:5254954- | + | 59 |
| 0059 | BG2 | UGAGAUAGU | 5250050 | | 5254974 | | |
| | | GU | | | | | |
| GCR- | HBG1/H | GGAAUGACU | chr11:5250102- | − | chr11:5255030- | − | 61 |
| 0061 | BG2 | GAAUCGGAA | 5250122 | | 5255050 | | |
| | | CA | | | | | |
| GCR- | HBG1/H | CUUGACCAA | chr11:5249957- | − | chr11:5254881- | − | 62 |
| 0062 | BG2 | UAGCCUUGA | 5249977 | | 5254901 | | |
| | | CA | | | | | |
| GCR- | HBG1/H | CAAGGCUAU | chr11:5249960- | + | chr11:5254884- | + | 63 |
| 0063 | BG2 | UGGUCAAGG | 5249980 | | 5254904 | | |
| | | CA | | | | | |
| GCR- | HBG1/H | AAGGCUGGC | chr11:5249979- | + | chr11:5254903- | + | 64 |
| 0064 | BG2 | CAACCCAUG | 5249999 | | 5254923 | | |
| | | GG | | | | | |
| GCR- | HBG1/H | ACUCGCUUC | chr11:5249835- | − | chr11:5254759- | − | 65 |
| 0065 | BG2 | UGGAACGUC | 5249855 | | 5254779 | | |
| | | UG | | | | | |
| GCR- | HBG1/H | AUUUGCAUU | chr11:5250031- | + | chr11:5254955- | + | 66 |
| 0066 | BG2 | GAGAUAGUG | 5250051 | | 5254975 | | |
| | | UG | | | | | |
| GCR- | HBG1/H | ACUGAAUCG | chr11:5250096- | − | chr11:5255024- | − | 67 |
| 0067 | BG2 | GAACAAGGC | 5250116 | | 5255044 | | |
| | | AA | | | | | |
| GCR- | HBG1/H | CCAUGGGUG | chr11:5249992- | + | chr11:5254916- | + | 68 |
| 0068 | BG2 | GAGUUUAGC | 5250012 | | 5254936 | | |
| | | CA | | | | | |
| GCR- | HBG1/H | AGAGUAUCC | chr11:5249914- | − | chr11:5254838- | − | 70 |
| 0070 | BG2 | AGUGAGGCC | 5249934 | | 5254858 | | |
| | | AG | | | | | |
| GCR- | HBG1/H | GAGUGUGUG | chr11:5249851- | + | chr11:5254775- | + | 71 |
| 0071 | BG2 | GAACUGCUG | 5249871 | | 5254795 | | |
| | | AA | | | | | |
| GCR- | HBG1/H | UAGUCUUAG | chr11:5249921- | − | chr11:5254845- | − | 72 |
| 0072 | BG2 | AGUAUCCAG | 5249941 | | 5254865 | | |
| | | UG | | | | | |

Additional preferred gRNAs comprise or consist of a targeting domain sequence of a) UUUGCCUUGUCAAGGCUAU (SEQ ID NO: 520), b) CUUGUCAAGGCUAUUGGUCA (SEQ ID NO: 53), c) CUUGACCAAUAGCCUUGACA (SEQ ID NO: 62), d) AAGGCUAUUGGUCAAGGCA (SEQ ID NO: 521), or. e) CUAUUGGUCAAGGCAAGGC (SEQ ID NO: 522), or a fragment thereof. The target sequences for these gRNAs are shown in Table 6.

In other preferred embodiments, the gene editing system, e.g., the CRISPR system, includes a gRNA which includes a targeting domain complementary to a target sequence at a target locus selected from the group consisting of: TET2, TRAC, TRBC1, TRBC2, CD3E, CD3G, CD3D, B2M, CIITA, CD247, HLA-A, HLA-B, HLA-C, DCK, CD52, FKBP1A, NLRC5, RFXANK, RFX5, RFXAP, NR3C1, CD274, HAVCR2, LAG3, PDCD1, PD-L2, CTLA4, CEACAM (e.g., CEACAM-1, CEACAM-3 and/or CEACAM-5), VISTA, BTLA, TIGIT, LAIR1, CD160, 2B4, CD80, CD86, B7-H3 (CD113), B7-H4 (VTCN1), HVEM (TNFRSF14 or CD107), KIR, A2aR, MHC class I, MHC class II, GAL9, adenosine, and TGF beta, PTPN11, and combinations thereof. Such target locuses include both intronic and exonic regions of said locus. In some embodiments, the target locus includes the coding region sequence(s) of one or more splice variants of said locus. In embodiments, the gene editing system including a CRISPR system including a gRNA molecule comprising a targeting domain described in PCT Publication WO/2017/093969, for example, described in any of Tables 1-6 and 6b-g of WO2017/093969. In embodiments, the cell to which the genome editing system is introduced is a T cell, and in preferred embodiments, the cell has been, is, or will be further engineered to express a chimeric antigen receptor, e.g., a chimeric antigen receptor as described in WO2017/093969 and the reference cited therein.

TALEN Gene Editing Systems

TALENs are produced artificially by fusing a TAL effector DNA binding domain to a DNA cleavage domain. Transcription activator-like effects (TALEs) can be engineered to bind any desired DNA sequence, e.g., a target gene. By combining an engineered TALE with a DNA cleavage domain, a restriction enzyme can be produced which is specific to any desired DNA sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is, for example, a wild-type or mutated FokI endonuclease. Several mutations to FokI have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. (2011) Nature Biotech. 29: 143-8.

A TALEN (or pair of TALENs) can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN, e.g., DNA encoding a transgene, and depending on the sequences of the foreign DNA and chromosomal sequence, this process can be used to integrate the transgene at or near the site targeted by the TALEN. TALENs specific to a target gene can be constructed using any method known in the art, including various schemes using modular components. Zhang et al. (2011) Nature Biotech. 29: 149-53; Geibler et al. (2011) PLoS ONE 6: e19509; U.S. Pat. Nos. 8,420,782; 8,470,973, the contents of which are hereby incorporated by reference in their entirety.

In embodiments, the gene editing system is as described in PCT Publication WO2015/073683. In embodiments, the gene editing system includes a TALEN system including a targeting domain complementary to any one of SEQ ID NO: 7-11, 16-62, and 143-184 of PCT Publication WO2015/073683.

Zinc Finger Nuclease (ZFN) Gene Editing Systems

“ZFN” or “Zinc Finger Nuclease” refer to a zinc finger nuclease, an artificial nuclease which can be used to modify, e.g., delete one or more nucleic acids of, a desired nucleic acid sequence.

Like a TALEN, a ZFN comprises a FokI nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5.

Also like a TALEN, a ZFN can create a double-stranded break in the DNA, which can create a frame-shift mutation if improperly repaired, leading to a decrease in the expression and amount of the target gene in a cell. ZFNs can also be used with homologous recombination to mutate the target gene or locus, or to introduce nucleic acid encoding a desired transgene at a site at or near the targeted sequence.

ZFNs specific to sequences in a target gene can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; and Guo et al. (2010) J. Mol. Biol. 400: 96; U.S. Patent Publication 2011/0158957; and U.S. Patent Publication 2012/0060230, the contents of which are hereby incorporated by reference in their entirety. In embodiments, The ZFN gene editing system may also comprise nucleic acid encoding one or more components of the ZFN gene editing system.

In embodiments of the invention the target sequence of a ZFN system includes at least the nucleic acid residues bound by one zinc finger protein. In other embodiments, particularly for ZFN systems comprising a two zinc finger nuclease proteins (e.g., dimeric systems), the target sequence comprises the nucleic acid sequence recognized by both of the zinc finger nuclease proteins. In embodiments, the target sequence additionally comprises the nucleic acids recognized by the nuclease domain.

In embodiments, the ZFN gene editing system is as described in PCT Publication WO2015/073683. In embodiments, the gene editing system comprises a ZFN system comprising a targeting domain complementary to any one of SEQ ID NO: 63-80 and 232-251 of PCT Publication WO2015/073683.

Meganuclease Gene Editing System

“Meganuclease” refers to a meganuclease, an artificial nuclease which can be used to edit a target gene.

Meganucleases are derived from a group of nucleases which recognize 15-40 base-pair cleavage sites. Meganucleases are grouped into families based on their structural motifs which affect nuclease activity and/or DNA recognition. Members of the LAGLIDADG family are characterized by having either one or two copies of the conserved LAGLIDADG motif (SEQ ID NO: 523) (see Chevalier et al. (2001), Nucleic Acids Res. 29(18): 3757-3774). The LAGLIDADG meganucleases with a single copy of the LAGLIDADG motif (SEQ ID NO: 523) form homodimers, whereas members with two copies of the LAGLIDADG motif (SEQ ID NO: 523) are found as monomers. The GIY-YIG family members have a GIY-YIG module, which is 70-100 residues long and includes four or five conserved sequence motifs with four invariant residues, two of which are required for activity (see Van Roey et al. (2002), Nature Struct. Biol. 9: 806-811). The His-Cys box meganucleases are characterized by a highly conserved series of histidines and cysteines over a region encompassing several hundred amino acid residues (see Chevalier et al. (2001), Nucleic Acids Res. 29(18): 3757-3774). The NHN family, the members are defined by motifs containing two pairs of conserved histidines surrounded by asparagine residues (see Chevalier et al. (2001), Nucleic Acids Res. 29(18): 3757-3774).

Strategies for engineering a meganuclease with altered DNA-binding specificity, e.g., to bind to a predetermined nucleic acid sequence are known in the art. E.g., Chevalier et al. (2002), Mol. Cell, 10:895-905; Epinat et al. (2003) Nucleic Acids Res 31: 2952-62; Silva et al. (2006) J Mol Biol 361: 744-54; Seligman et al. (2002) Nucleic Acids Res 30: 3870-9; Sussman et al. (2004) J Mol Biol 342: 31-41; Rosen et al. (2006) Nucleic Acids Res; Doyon et al. (2006) J. Am Chem Soc 128: 2477-84; Chen et al. (2009) Protein Eng Des Sel 22: 249-56; Arnould S (2006) J Mol Biol. 355: 443-58; Smith (2006) Nucleic Acids Res. 363(2): 283-94.

A meganuclease can create a double-stranded break in the DNA, which can create a frame-shift mutation if improperly repaired, e.g., via non-homologous end joining, leading to a decrease in the expression of a target gene in a cell. Alternatively, foreign DNA can be introduced into the cell along with the Meganuclease; depending on the sequences of the foreign DNA and chromosomal sequence, this process can be used to modify a target gene, e.g., correct a defect in the target gene, thus causing expression of a repaired target gene, or e.g., introduce such a defect into a wt gene, thus decreasing expression of a target gene, e.g., as described in Silva et al. (2011) Current Gene Therapy 11:11-27.

Methods of Treatment

The present invention provides methods of treating patients suffering from a disorder with a gene editing system, whereby the decision to treat the patient is made based on assaying for the presence of the target sequence, at the target locus, recognized by the targeting domain of the gene editing system. In embodiments, presence of a fully complementary target sequence at the target sequence indicates that the patient is to be treated with the gene editing system. In embodiments, treatment of the patient with the gene editing system includes administering the gene editing system to the patient (sometimes referred to as in vivo gene editing therapy). In other embodiments, treatment of the patient with the gene editing system involves administration of the gene editing system to a population of cells, e.g., a population of cells provided ex vivo, and then subsequent administration of the cells to the patient. In embodiments, the cells are autologous to the patient to which they are administered. In embodiments, the cells are allogeneic (e.g., derived from a healthy human donor) to the patient to which they are administered.

In an aspect, the invention provides a method of selectively treating a patient with a gene editing system, including:
c) selectively introducing said gene editing system into a cell, e.g., population of cells, of the patient on the basis of the cell, e.g., population of cells, comprising a target sequence, at a target locus, that is fully complementary to a targeting domain of said gene editing system; and/or
d) selectively introducing said gene editing system to a cell, e.g., population of cells, of the patient on the basis of the cell, e.g., population of cells, not comprising a target sequence, at a locus other than the target locus, that is fully complementary to a targeting domain of said gene editing system.

In an aspect, the invention provides a method of selectively treating a patient with a gene editing system, including:
a) selecting the patient for treatment on the basis of one or more cells of the patient comprising a target sequence, at a target locus, that is fully complementary to a targeting domain of said gene editing system; and
b) thereafter, administering a therapeutically effective amount of said gene editing system to the patient or to a population of cells of said patient,
thereby inducing a modification at or near the target sequence at the target locus in a cell or the patient or a cell of the population of cells.

In an aspect, the invention provides a method of selectively treating a patient with a gene editing system including:
a) assaying one or more cells from a biological sample from the patient for the presence of a target sequence, at a target locus, that is fully complementary to a targeting domain of said gene editing system; and
b) thereafter, selectively administering a therapeutically effective amount of the gene editing system to the patient or to a cell of the patient:
   i) on the basis of one or more cells of the biological sample of the patient comprising a target sequence, at a target locus, that is fully complementary to a targeting domain of said gene editing system; and/or
   ii) on the basis of one or more cells of the biological sample from the patient not comprising a target sequence, at a locus other than the target locus, that is fully complementary to a targeting domain of said gene editing system,

thereby inducing a modification at or near the target sequence at the target locus in a cell or the patient or a cell of the population of cells.

In an aspect, the invention provides a method of selectively treating a patient with a gene editing system, including:
a) assaying one or more cells of a biological sample from the patient for at least one target sequence, at a target locus, that is fully complementary to the targeting domain of said gene editing system;
b) thereafter, selecting the patient for treatment with the gene editing system on the basis of one or more cells of the biological sample from the patient having the target sequence, at the target locus, that is fully complementary to the targeting domain of said gene editing system; and
c) thereafter, administering a therapeutically effective amount of the gene editing system of cells to the patient.

In an aspect, the invention provides a gene editing system for use in treating a patient having a disease, characterized in that a therapeutically effective amount of the gene editing system is to be administered to the patient (or cells of the patient) on the basis of a cell of said patient comprising a target sequence, at a target locus, that is fully complementary to a targeting domain of said gene editing system.

In an aspect, the invention provides a gene editing system for use in treating a patient having a disease, characterized in that:
a) the patient is to be selected for treatment with the gene editing system on the basis of a cell of said patient comprising a target sequence, at a target locus, that is fully complementary to a targeting domain of said gene editing system; and
b) thereafter, a therapeutically effective amount of the gene editing system is to be administered to the patient.

In an aspect, the invention provides a gene editing system for use in treating a patient having a disease, characterized in that:
a) a cell of a biological sample from the patient is to be assayed for at least one a target sequence, at a target locus, that is fully complementary to a targeting domain of said gene editing system; and
b) a therapeutically effective amount of the gene editing system is to be selectively administered to the patient on the basis of the cell of the biological sample from the patient having the at least one a target sequence, at a target locus, that is fully complementary to a targeting domain of said gene editing system.

In an aspect, the invention provides a gene editing system for use in treating a patient having a disease, characterized in that:
c) a cell of a biological sample from the patient is to be assayed for at least one a target sequence, at a target locus, that is fully complementary to a targeting domain of said gene editing system;
d) the patient is selected for treatment with the gene editing system on the basis of the cell of the biological sample from the patient having the at least one a target sequence, at a target locus, that is fully complementary to a targeting domain of said gene editing system; and
c) a therapeutically effective amount of the gene editing system is to be selectively administered to the patient.

In an aspect, the invention provides a method of predicting the likelihood that a patient having an disease will respond to treatment with a gene editing system, comprising assaying a cell of a biological sample from the patient for the presence or absence of at least one target sequence, at a target locus, that is fully complementary to a targeting domain of said gene editing system, wherein:
a) the presence of the at least one target sequence, at a target locus, that is fully complementary to a targeting domain of said gene editing system is indicative of an increased likelihood that the patient will respond to treatment with the gene editing system; and
b) the absence of the at least one target sequence, at a target locus, that is fully complementary to a targeting domain of said gene editing system is indicative of a decreased likelihood that the patient will respond to treatment with the gene editing system.

In embodiments, the method further includes the step of obtaining the biological sample from the patient, wherein the step of obtaining is performed prior to the step of assaying.

In aspects of the invention, the cells (or population of cells) assayed for the presence of the fully complementary target sequence at the target locus are of a cell type intended to be modified by the gene editing system. In embodiments, the cells are mammalian, for example, human. In embodiments, the cells include, e.g., consist of, hematopoietic stem and progenitor cells (HSPCs) or HSCs. In other embodiments, the cells include, e.g., consist of, immune effector cells, e.g., T cells or NK cells, e.g., T cells.

In aspects the disease to be treated is a rare metabolic disorder. In an aspect the disease to be treated is a cancer or autoimmune disease. In an aspect the disease to be treated is a hemoglobinopathy, for example, sickle cell disease, sickle cell anemia, beta-thalassemia, thalassemia major, thalassemia intermedia.

In embodiments where cells to be assayed are derived from a biological sample, the biological sample may be selected from the group consisting of synovial fluid, blood, bone marrow, serum, feces, plasma, urine, tear, saliva, cerebrospinal fluid, an apheresis sample, a leukopheresis sample, a leukocyte sample and a tissue sample.

Methods for Ascertaining the Target Sequence at the Target Locus

A variety of techniques are known in the art for sequencing a target locus (e.g., for ascertaining the presence or absence of a target sequence at a target locus). Such methods include technique such as Next generation sequencing (NGS), pyrosequencing, Sanger sequencing, Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southern Blot, immunoassays, immunohistochemistry, ELISA, flow cytometry, Western blot, HPLC, and mass spectrometry.

Other methods, including preferred methods, include, for example, Deoxyribonucleic acid sequencing (Sanger Sequencing); Next generation sequencing (NGS); Pyrosequencing; Polymerase chain reaction (PCR) and its modified versions, for example, Reverse-transcriptase PCR analysis, Real time PCR (Real-time PCR 4th Edition. (http://find.thermofisher.com/qpcr/real-pcr-handbook/merch), or Amplification refractory mutation system (ARMS) PCR (Newton C R, Graham A, Heptinstall L E, et al. Analysis of any point mutation in DNA. The amplification refractory mutation system (ARMS) Nucleic Acids Res. 1989; 17(7):2503-16); Microarray (Kothiyal, P. et al. An Overview of Custom Array Sequencing. Curr Protoc Hum Genet. 2009 April; 0 7: Unit-7.17); Multiplex ligation-dependent probe amplification (MLPA) (Taylor C F, Charlton R S, Burn J, et al. Genomic deletions in MSH2 or MLH1 are a frequent cause of hereditary non-polyposis colorectal cancer: Identification of novel and recurrent deletions by MLPA. Hum Mutat. 2003; 22(6):428-33); Single-strand conformation polymorphism analysis (SSCP) (Kakavas V K, Plageras P, Vlachos T A, et al. PCRSSCP: A method for the molecular analysis of genetic diseases. Mol Biotechnol. 2008; 38(2): 155-63); Heteroduplex analysis (Glavac D, Dean M. Applications of heteroduplex analysis for mutation detection in disease genes. Hum Mutat. 1995; 6(4):281-7); Denaturing Gradient Gel Electrophoresis (DGGE) (Fodde R, Losekoot M. Mutation detection by denaturing gradient gel electrophoresis (DGGE) Hum Mutat. 1994; 3(2):83-94); Restriction fragment length polymorphism (RFLP); MALDI-TOF mass spectrometry (Jurinke, C. et al. MALDI-TOF mass spectrometry: a versatile tool for high-performance DNA analysis. Mol. Biotechnol. 26, 147-163 (2004)); Denaturing high-performance liquid chromatography (DHPLC) (Fackenthal D L, Chen P X, Das S. (2005). Denaturing high-performance liquid chromatography for mutation detection and genotyping. Methods Mol Biol. 311:73-96, Yu B, Sawyer N A, Chiu C, Oefner P J, Underhill P A. (2006) DNA mutation detection using denaturing high-performance liquid chromatography (DHPLC). Curr Protoc Hum Genet. Chapter 7:Unit7.10); High resolution melting (HRM) analysis (A Guide to High Resolution Melting (HRM) Analysis. (Applied Biosystems); Do H., Krypuy, M et al. (2008). High resolution melting analysis for rapid and sensitive EGFR and KRAS mutation detection in formalin fixed paraffin embedded biopsies BMC Cancer 8:142)

Preferred embodiments rely on NGS, e.g., NGS as described in Example 2, or Sanger sequencing, e.g., Sanger sequencing as described in Example 3, or Pyrosequencing, e.g., Pyrosequencing as described in Example 4.

### EXAMPLES

### Example 1: Cutting Efficiency (Indel %) is Substantially Reduced at Target Sequences with Mismatches

The likelihood of an in silico identified off-target site being actively edited is inversely proportional to the total number of mismatches in the off-target site i.e. the lower the number of mismatches the higher risk of editing. However there are currently no precise rules of predicting which in silico identified off-target sites are active. In line with previous reports (PMID: 26189696, PMID: 23907171, incorporated herein by reference in their entireties) table 4 shows that the gRNAs designed to target regions within the BCL11a enhancer or within an HPFH region (as measured by indel formation by NGS) exhibit high cutting frequency at fully complementary target sequences, but exhibit substantially reduced cutting efficiency at target sequences containing mismatches. In the majority of cases having as few as 2 mismatches, there was no detectable editing. For example for gRNA CR001028 the on-target editing efficiency is approximately 92%, whereas the only in silico define 2 mismatch off-target site only has an editing efficiency of approximately 3%. Table 4 also shows that all one mismatch in silico identified off-target sites show editing, however the editing efficiencies are typically lower than the on-target site. For example for gRNA GCR-0051, the off-target editing efficiency for the single one mismatch off-target site identified is approximately 40% compared to the on-target editing efficiency of approximately 88%.

TABLE 4
In silico identified off-target sites for the +58 BCL11a erythroid specific enhancer
(ESH) region, HBD, HBB region gRNAs with 1 or 2 mismatches showing off-target
sequence, genomic location, number of mismatches, and approximate editing efficiency.
Approximate on-target editing efficiency for each gRNA is also shown.

| | | On- | | | | | Off- |
|---|---|---|---|---|---|---|---|
| | gRNA | target | Off-target | SEQ | hg38 genomic | No. of | target |
| Locus | name | activity | sequence | ID NO: | coordinates | mismatches | activity |
| +58 | CR0 | 92% | CACGaCCCa | 524 | chr1:236,065,4 | 2 | 80% |
| BCL1 | 0309 | | ACCCTAATC | | 15- | | |
| 1a | | | AG | | 236,065,434 | | |
| ESH | | | CACGCCCaC | 525 | chr12:3,311,90 | 2 | 18% |
| | | | ACCtTAATC | | 7-3,311,926 | | |
| | | | AG | | | | |
| | CR0 | 95% | TTTGGCCTag | 526 | chr3:16,428,54 | 2 | ND |
| | 0311 | | GATTAGGGT | | 6-16,428,565 | | |
| | | | G | | | | |
| | | | TTTGGCCTg | 527 | chr15:76,666,2 | 2 | ND |
| | | | TGAgTAGGG | | 68-76,666,287 | | |
| | | | TG | | | | |
| | | | TgTGGCCTC | 528 | chr16:55,159,1 | 2 | ND |
| | | | TGATTAGGa | | 91-55,159,210 | | |
| | | | TG | | | | |
| | CR0 | 91% | TTTTgTCAC | 529 | chr2:206,371,5 | 2 | ND |
| | 0112 | | AGGCTCCAG | | 38- | | |
| | 5 | | tA | | 206,371,557 | | |
| | CR0 | 92% | TTTATCACAt | 530 | chr6:1,188,012- | 2 | ND |
| | 0112 | | GCTCCAGGA | | 1,188,031 | | |
| | 6 | | c | | | | |
| | | | TTTATaACA | 531 | chr4:35,881,81 | 2 | 2% |
| | | | GGCTCCAGa | | 5-35,881,834 | | |
| | | | AA | | | | |
| | CR0 | 97% | CACAGGCTC | 532 | chr7:158,190,4 | 2 | ND |
| | 0112 | | CtGGAAGGc | | 53- | | |
| | 7 | | TT | | 158,190,472 | | |
| HPFH | CR0 | 91% | TGgGGTGGG | 533 | chr4:58,169,30 | 2 | 3% |
| HBD | 0102 | | GAGATATGa | | 8-58,169,327 | | |
| | 8 | | AG | | | | |
| | CR0 | 85% | GcAAGCATT | 534 | chr17:49,013,5 | 2 | ND |
| | 0113 | | TAAGTGGCa | | 54-49,013,573 | | |
| | 7 | | AC | | | | |
| | CR0 | 95% | GAAACAAT | 535 | chr11:80,808,1 | 2 | ND |
| | 0122 | | GAGGACCT | | 71-80,808,190 | | |
| | 1 | | GtgT | | | | |
| | | | GAAACAcTG | 536 | chr2:210,493,4 | 2 | ND |
| | | | AGcACCTGA | | 33- | | |
| | | | CT | | 210,493,452 | | |
| | CR0 | 89% | AGGCACCTC | 537 | chr2:37,780,20 | 2 | ND |
| | 0303 | | AGACTgAGC | | 7-37,780,226 | | |
| | 5 | | Ac | | | | |
| | | | AGGCACCTC | 538 | chr6:5,335,984 | 2 | ND |
| | | | AGAtTCAGC | | -5,336,003 | | |
| | | | Ac | | | | |
| | | | AGGCACCTC | 539 | chrX:129,302, | 2 | ND |
| | | | AcACTCAaC | | 657- | | |
| | | | AT | | 129,302,676 | | |
| | | | AGGtACCTC | 540 | chr2:66,281,78 | 2 | 20% |
| | | | AaACTCAGC | | 1-66,281,800 | | |
| | | | AT | | | | |
| HPFH | GCR | 58% | AGTCCTGGT | 541 | chr11:5,255,09 | 2 | ND |
| HBG | - | | ATCtTCTATG | | 8-5,255,117 | | |
| | 0001 | | g | | | | |
| | | | AGcCCTGGTt | 542 | chr1:5,184,221 | 2 | ND |
| | | | TCCTCTATG | | -5,184,240 | | |
| | | | A | | | | |
| | | | AGTCCTGGa | 543 | chr14:18,502,2 | 2 | NS |
| | | | ATCCTaTAT | | 83-18,502,305 | | |
| | | | GA | | | | |
| | | | AGTCCTGGa | 543 | chr22:15,429,8 | 2 | NS |
| | | | ATCCTaTAT | | 14-15,429,836 | | |
| | | | GA | | | | |
| | GCR | 89% | GGAGAAGaA | 544 | chr11:5,255,07 | 1 | 85% |
| | - | | AACTAGCTA | | 6-5,255,095 | | |
| | 0008 | | AA | | | | |
| | GCR | 30% | GGGAGAAGa | 545 | chr11:5,255,07 | 1 | 27% |
| | - | | AAACTAGCT | | 7-5,255,096 | | |
| | 0010 | | AA | | | | |
| | GCR | 88% | GGAGAAGA | 546 | chr10:82,509,1 | 2 | ND |
| | - | | AAACTAGtT | | 65-82,509,184 | | |
| | 0051 | | AgA | | | | |
| | | | GGtGAAaAA | 547 | chr2:81,090,78 | 2 | ND |
| | | | AACTAGCTA | | 3-81,090,802 | | |
| | | | AA | | | | |
| | | | GGAGAAGA | 548 | chr6:100,995,2 | 2 | ND |
| | | | AAAaTAGCT | | 15- | | |
| | | | gAA | | 100,995,234 | | |
| | | | GGAGAAGg | 549 | chr11:5,250,14 | 1 | 40% |
| | | | AAACTAGCT | | 5-5,250,167 | | |
| | | | AAA | | | | |

ND = none detected,
NS = not screened

In addition, PMID: 24115442 (incorporated herein by reference in its entirety) reported genetic variation within the +58 region, and single nucleotide polymorphisms are known to exist throughout the genome, particularly in non-coding regions such as introns, promoters and intragenic regions. These findings, together with the data shown in Table 4 suggests that therapies which utilize sequence specific cutting of target sequences using, for example, genome editing systems such as CRISPR systems described herein, will be most effective when a target sequence which is fully complementary to the targeting domain of the gene editing system is present at the locus of interest. Thus, the invention provides methods, for example as described herein, for selecting patients for treatment with a genome editing system comprising assaying for the presence of a fully complementary target sequence at the target locus within the patient of interest, and treating said patient on the basis of such information (as more fully described herein).

### Example 2: Protocol for Assaying the Target Sequence: Amplicon Based Illumina Sequencing (NGS)

During the past 15 years, a number of next generation sequencing (NGS) technologies have been developed, allowing sequencing millions of DNA molecules in parallel. Major commercially available high throughput NGS technologies include, 454 pyrosequencing, Illumina sequencing, SOLiD sequencing, PACBIO R S, HeliScope sequencing, Ion Torrent and Oxford Nanopore technologies. Among those, Illumina sequencing by synthesis (SBS) chemistry is the most widely adopted chemistry. The principle of Illumina sequencing technologies is similar to Sanger sequencing, while the critical difference is that it is able to sequence millions of DNA molecules simultaneously. Numerous Illumina NGS protocols exist to determine target nucleotide information, while sequencing methods differ primarily by how the DNA or RNA samples are processed and by the data analysis options used. Below is one procedure for performing NGS:
1. Forward and reverse PCR primers complementary to a sequences proximal (e.g., within 200, 150, 100 or 50, preferably within about 100 nucleotides) to the target sequence are designed and synthesized with down-stream flanking sequence and illumine-specified overhang adapters using Primer 3 (https://www.ncbi.nlm.nih.gov/tools/primer-blast/) or other equivalent primer design tool (e.g. http://www.idtdna.com/calc/analyzer).
2. PCR is performed using 2×KAPA HiFi HotStart ReadyMix (Kapa biosystems) with DNA template derived from the patient's cells (e.g., the cells of interest to be edited) and PCR primers designed in step 1.
3. PCR products are purified by using AMPure kit (Agencourt Bioscience Corporation, Beverly, Mass.).
4. Purified PCR products are attached to dual indices and Illumina sequencing adapters using the Nextera XT Index Kit (Illumina).
5. Sequencing library from step 4 is subjected to the following steps before Illumina sequencing on an Illumina NGS system, such as MiSeq: clean up, quantification, normalization and denaturalization.
6. Sequencing data can be processed and aligned to reference sequence by SAMTOOLS and BWA or other equivalent NGS software.
NGS sequencing is also described in, for example, Levy, S E and Myers, R M (2016). Advancements in Next-Generation Sequencing. Annual Review of Genomics and Human Genetics. 17:95-115; Mardis, E R. (2013) Next-Generation Sequencing Platforms. Annu. Rev. Anal. Chem. 6:287-303; Goodwin, S., McPherson, J D, McCombie, W R. (2016). Coming of age: ten years of next-generation sequencing technologies. Nature Reviews Genetics 17: 33-351 (and references cited therein); Mardis, E., Next generation DNA sequencing methods. Ann. Rev. Genomics Hum. Genet, 9: 387-402 (2008); Shendure, J. and Ji, H., Next-generation DNA sequencing. Nat. Biotechnol., 26: 1135-1145 (2008); and https://www.illumina.com/content/dam/illumina-marketing/documents/products/illumina_sequencing_introduction.pdf; and/or https://support.illumina.com/content/dam/illumina-support/documents/documentation/chemistry_documentation/16s/16s-metagenomic-library-prep-guide-15044223-b.pdf, the contents of which are hereby incorporated by reference in their entireties.

Once the locus of interest is sequenced, the sequence is compared against the fully complementary target sequence, and the patient assessed for treatment with a genome editing system comprising a targeting domain fully complimentary to the target sequence based on the sequencing information. For example, if the patient's cell, e.g., cell of the cell type to be genome edited, contains a sequence which is identical to the target sequence fully complementary to the targeting domain sequence of the genome editing system, the patient is identified as having a high likelihood of response to the genome editing system therapy, and is treated with the genome editing system.

### Example 3: Protocol for Assaying the Target Sequence: Sanger Sequencing

Sequencing information has traditionally been determined using Sanger sequencing. One such method is based on polymerase termination with fluorescent dideoxynucleotides followed by sequence collection on automated capillary electrophoresis (CE) instruments.

Experimental Protocol for Sanger Sequencing:
1. Forward and reverse PCR primers complementary to sequences proximal (e.g., within 200, 150, 100 or 50, preferably within about 100 nucleotides) to the target sequence are designed using Primer 3. (https://www.ncbi.nlm.nih.gov/tools/primer-blast/) or other equivalent primer design tool (e.g. http://www.idtdna.com/calc/analyzer).
2. PCR is performed in 10 μl reactions by using Advantage®-HF2 PCR kit (Clontech, Mountain View, Calif.), containing 1 μl 10×HF 2 PCR Buffer, 1 μl 10×HF 2 dNTP Mix, 0.2 μl polymerase, 2 μl genomic DNA (50 ng/ul), 0.4 μl forward primer (10 μM), 0.4 μl reverse primer (10 μM), 1 ul DMSO (Thermo Fisher Scientific, Waltham, Mass.) and 4 μl ddH₂O.
3. Reactions are carried out in 384-well GeneAmp® 9700 thermocyclers (Applied Biosystems, Foster City, Calif.) using a touchdown PCR protocol (1 cycle of 94° C. for 1 min; 5 cycles of 94° C. for 20 sec, 60° C. for 20 sec (decrease 1° C. per cycle to 55° C.), 68° C. for 1 min; 25 cycles of 94° C. for 20 sec, 55° C. for 20 sec, 68° C. for 1 min; 1 cycle of 68° C. for 5 min).
4. 10 μl PCR products are purified and eluted with 30 μl ddH₂O after 5 min incubation by using AMPure kit (Agencourt Bioscience Corporation, Beverly, Mass.).
5. Forward and reverse sequencing primers are designed using Primer 3. (https://www.ncbi.nlm.nih.gov/tools/primer-blast/).
6. Sequencing reactions are carried out with sequencing primer and BigDye® Terminator v.1.1 Cycle Kit (Applied Biosystems). The sequencing reactions are set up as the following: 1.75 μl 5× sequencing buffer (Applied Biosystems), 0.5 μl BigDye® v1.1 Cycle terminator (Applied Biosystems), 1 μl sequencing primer, 4.75 μl ddHO, and 2 μl AMPure purified PCR product. Sequencing reactions are performed in a 384-well GeneAmp® 9700 thermocycler as the following: 1 cycle of 96° C. for 10 sec; 25 cycles of 96° C. for 10 sec, 50° C. for 10 sec, 60° C. for 1 min; 4° C. hold). Afterwards, sequencing products are purified and eluted with 30 μl ddH₂O after 5 min incubation by using the CleanSEQ kit (Agencourt Bioscience Corporation).
7. Sequencing fragments are detected via capillary electrophoresis using an ABI PRISM 3730xl DNA analyzer (Applied Biosystems).
8. Target sequencing data is analyzed using software Sequencher (Gene Codes Corporation), or Phred, Phrap, Consed (University of Washington) or other equivalent sequencing analysis tools.

Sanger sequencing is additionally described at, for example, Sanger F., Nicklen S., and Coulson A R. (1977) DNA sequencing with chain-terminating inhibitors. Proc Natl Acad Sci USA. 74(12): 5463-7; Smith L M, Sanders J Z, Kaiser R J, et al. (1986). “Fluorescence detection in automated DNA sequence analysis”. Nature. 321 (6071): 674-9; BigDye™ Terminator v1.1 Cycle Sequencing Kit USER GUIDE. Applied Biosystems (https://tools.thermofisher.com/content/sfs/manuals/cms_041330.pdf), the contents of which are hereby incorporated by reference in their entireties.

Once the locus of interest is sequenced, the sequence is compared against the fully complementary target sequence, and the patient assessed for treatment with a genome editing system comprising a targeting domain fully complimentary to the target sequence based on the sequencing information. For example, if the patient's cell, e.g., cell of the cell type to be genome edited, contains a sequence which is identical to the target sequence fully complementary to the targeting domain sequence of the genome editing system, the patient is identified as having a high likelihood of response to the genome editing system therapy, and is treated with the genome editing system.

### Example 4: Pyrosequencing

Pyrosequencing is a sequencing method based on sequencing-by-synthesis, first described by Ronaghi, et al (Ronaghi, M., M. Uhlén and P. Nyrén. 1998. A sequencing method based on real-time pyrophosphate. Science 281:363, 365). Details of the sequencing principle are described at a website established by Qiagen (https://www.qiagen.com/be/resources/technologies/pyrosequencing-resource-center/technology-overview/). In brief, it relies on the detection of PPi that is released upon nucleotide incorporation by using a four-enzyme mixture, DNA polymerase, ATP sulfurylase, luciferase, and apyrase, as well as the substrates adenosine 5′ phosphosulfate (APS). The released PPi is converted to adenosine triphosphate (ATP) by ATP sulfurylase, which, in turn, can be detected by luciferase to generate a visible light in amounts that are proportional to the amount of ATP. The light produced in the luciferase-catalyzed reaction is detected by CCD sensors and recorded as a peak in the raw data output (Pyrogram). Unreacted nucleotides are subsequently degraded by apyrase to allow the cyclic addition of nucleotides to the reaction system. As the process continues, the complementary DNA strand is elongated and the nucleotide sequence is determined from the signal peaks from the Pyrogram trace.

### Example 5: Analysis of Human Variation in Genomic Sequence Targeted by Gene Editing Systems

To investigate the known prevalence of variant sequences of target sequences of gene editing systems, we used publically available whole genome sequencing data to determine what, if any, naturally occurring genetic variation are found in the target sequences bound by gene editing systems. The data used was from phase 3 of the 1000 Genomes Project [A global reference for human genetic variation, The 1000 Genomes Project Consortium, Nature 526, 68-74 (1 Oct. 2015) doi: 10.1038/nature15393], the African Genome Variation Project (AGVP) [The African Genome Variation Project shapes medical genetics in Africa, Gurdasani, D. et al, Nature 517, 327-332 (15 Jan. 2015) doi: 10.1038/nature13997], and Genome Aggregation Database [Analysis of protein-coding genetic variation in 60,706 humans, Monkol Lek, Konrad J. Karczewski et al., Exome Aggregation Consortium, Nature 536, 285-291 (18 Aug. 2016) doi: 10.1038/nature19057]. Combined, these data include more than fifteen thousand whole genome sequences, of which more than four thousand are from individuals with African ancestry representing 14 populations (see Table 5). At a base pair resolution we searched the resources for reported deviations from the human reference genome (which includes the fully complementary target sequence of each gene editing system assessed) at the genomic target sequences recognized by the gRNA molecule's targeting domain. The target sequences assessed are listed in Table 6. We observed no variation above an allele frequency of 0.01 in the available data in the target sequences tested. However, variant sequences of several of the target sequences were identified at the respective alleles at frequencies below the 0.01 threshold. These variants, and their frequencies in the data sets are shown in Table 7.

To confirm our findings that the on-target sites lack significant deviations from the target region we will perform targeted genomic sequencing of the guide region as an inclusion criteria.

TABLE 5
Number of individual genomes in each database evaluated.

| | Genomes | |
|---|---|---|
| | | Individuals with |
| Source | Total | African ancestry |
| AGVP | 320 | 320 |
| Genome aggregation database | 15,496 | 4,368 |
| 1000 genomes project | 3,500 | 1,018 |

TABLE 6
Genomic localization of target sequence of gRNA molecules

| | | | gRNA target | | | | | | |
|---|---|---|---|---|---|---|---|---|---|
| | gRNA | | sequence (with | | | | | | |
| | Targeting | SEQ | PAM shown in | SEQ | c | | | | as- |
| Identi- | Domain | ID | lowercase | ID | h | | | | sem- |
| fier | Sequence | NO: | letters) | NO: | r | strand | start | stop | bly |
| gRNA | UUUGCCUU | 520 | TTTGCCTTGT | 550 | 1 | + | 5271 | 5271 | hg19 |
| 01 | GUCAAGGC | | CAAGGCTATt | | 1 | | 181 | 200 | |
| | UAU | | gg | | | | | | |
| gRNA | UUUGCCUU | 520 | TTTGCCTTGT | 550 | 1 | + | 5276 | 5276 | hg19 |
| 01 | GUCAAGGC | | CAAGGCTATt | | 1 | | 105 | 124 | |
| | UAU | | gg | | | | | | |
| gRNA | CUUGUCAA | 53 | CTTGTCAAG | 551 | 1 | + | 5271 | 5271 | hg19 |
| 02 | GGCUAUUG | | GCTATTGGT | | 1 | | 186 | 205 | |
| | GUCA | | CAagg | | | | | | |
| gRNA | CUUGUCAA | 53 | CTTGTCAAG | 551 | 1 | + | 5276 | 5276 | hg19 |
| 02 | GGCUAUUG | | GCTATTGGT | | 1 | | 110 | 129 | |
| | GUCA | | CAagg | | | | | | |
| gRNA | CUUGACCA | 62 | CTTGACCAA | 552 | 1 | − | 5271 | 5271 | hg19 |
| 03 | AUAGCCUU | | TAGCCTTGA | | 1 | | 188 | 207 | |
| | GACA | | CAagg | | | | | | |
| gRNA | CUUGACCA | 62 | CTTGACCAA | 552 | 1 | − | 5276 | 5276 | hg19 |
| 03 | AUAGCCUU | | TAGCCTTGA | | 1 | | 112 | 131 | |
| | GACA | | CAagg | | | | | | |
| gRNA | AAGGCUAU | 521 | AAGGCTATT | 553 | 1 | + | 5271 | 5271 | hg19 |
| 04 | UGGUCAAG | | GGTCAAGGC | | 1 | | 192 | 211 | |
| | GCA | | Aagg | | | | | | |
| gRNA | AAGGCUAU | 521 | AAGGCTATT | 553 | 1 | + | 5276 | 5276 | hg19 |
| 04 | UGGUCAAG | | GGTCAAGGC | | 1 | | 116 | 135 | |
| | GCA | | Aagg | | | | | | |
| gRNA | CUAUUGGU | 522 | CTATTGGTC | 554 | 1 | + | 5271 | 5271 | hg19 |
| 05 | CAAGGCAA | | AAGGCAAGG | | 1 | | 196 | 217 | |
| | GGC | | Ctgg | | | | | | |
| gRNA | CUAUUGGU | 522 | CTATTGGTC | 554 | 1 | + | 5276 | 5276 | hg19 |
| 05 | CAAGGCAA | | AAGGCAAGG | | 1 | | 120 | 141 | |
| | GGC | | Ctgg | | | | | | |
| CR000 | CUAACAGU | 253 | CTAACAGTT | 555 | 2 | − | 6072 | 6072 | hg19 |
| 317 | UGCUUUUA | | GCTTTTATCA | | | | 2396 | 2418 | |
| | UCAC | | Cagg | | | | | | |
| GCR- | ACUGAAUC | 67 | ACTGAATCG | 556 | 1 | − | 5271 | 5271 | hg19 |
| 067 | GGAACAAG | | GAACAAGGC | | 1 | | 324 | 346 | |
| | GCAA | | AAagg | | | | | | |
| GCR- | ACUGAAUC | 67 | ACTGAATCG | 556 | 1 | − | 5276 | 5276 | hg19 |
| 067 | GGAACAAG | | GAACAAGGC | | 1 | | 252 | 274 | |
| | GCAA | | AAagg | | | | | | |
| CR001 | AUCAGAGG | 338 | ATCAGAGGC | 557 | 2 | + | 6072 | 6072 | hg19 |
| 128 | CCAAACCC | | CAAACCCTT | | | | 2371 | 2393 | |
| | UUCC | | CCtgg | | | | | | |

TABLE 7
Variant sequences identified

| source | gRNA Id | chr | pos | id | ref | alt | qual | AF |
|---|---|---|---|---|---|---|---|---|
| AGVP | CR00245 | 2 | 60721618 | rs976776743 | T | G | NA | NA |
| gnom AD | CR001128 | 2 | 60722379 | rs769705137 | C | G | 2889.05 | 0.0001616450 |
| gnom AD | CR000317 | 2 | 60722403 | rs912402635 | T | G | 348.46 | 0.0000323081 |
| gnom AD | CR000317 | 2 | 60722418 | . | G | C | 166.46 | 0.0000323081 |
| 1KGP | CR001028 | 11 | 5255912 | rs3813727 | A | G | 100 | 0.4918130000 |
| AGVP | CR001028 | 11 | 5255912 | rs3813727 | A | G | NA | NA |
| gnom AD | CR001028 | 11 | 5255912 | rs381727 | A | G | 8185239.87 | 0.4144980000 |
| 1KGP | CR001028 | 11 | 5255926 | rs190495739 | C | T | 100 | 0.0013977600 |
| gnom AD | CR001028 | 11 | 5255926 | rs190495739 | C | T | 66508.71 | 0.0034224500 |
| gnom AD | CR001028 | 11 | 5255927 | rs761746243 | G | A | 471.48 | 0.0000322977 |
| gnom AD | CR001137 | 11 | 5257149 | rs914973791 | G | C | 366.4 | 0.0000646454 |
| 1KGP | CR001137 | 11 | 5257153 | rs76445361 | C | T | 100 | 0.0189696000 |
| AGVP | CR001137 | 11 | 5257153 | rs76445361 | C | T | NA | NA |
| gnom AD | CR001137 | 11 | 5257153 | rs76445361 | C | T | 232319.59 | 0.0180984000 |
| 1KGP | CR001137 | 11 | 5257165 | rs572417936 | c | T | 100 | 0.0001996810 |
| 1KGP | CR003035 | 11 | 5258728 | rs111334276 | G | A | 100 | 0.0043929700 |
| gnom AD | CR003035 | 11 | 5258728 | rs111334276 | G | A | 63531.88 | 0.0057141000 |
| gnom AD | CR003035 | 11 | 5258729 | . | T | C | 170.47 | 0.0000323039 |
| gnom AD | gRNA01 | 11 | 5271185 | . | C | G | 164.55 | 0.0000467202 |
| gnom AD | GCR_067 | 11 | 5271334 | . | G | A | 126.51 | 0.0000335864 |
| gnom AD | GCR_067 | 11 | 5271337 | . | C | G | 247.5 | 0.0000333533 |
| gnom AD | GCR_067 | 11 | 5271339 | rs954794288 | G | A | 315.52 | 0.0000332491 |
| 1KGP | GCR_067 | 11 | 5276266 | rs112511765 | C | T | 100 | 0.0001996810 |
| gnom AD | GCR_067 | 11 | 5276266 | rs112511765 | C | T | 583.41 | 0.0000645453 |
| gnom AD | GCR_067 | 11 | 5276267 | rs1045222350 | G | A | 441.41 | 0.0000322747 |

| source | AF_AFR | AF_EUR | AF_NFE | AF_POPMAX |
|---|---|---|---|---|
| AGVP | 0.0031250000 | NA | NA | NA |
| gnom AD | NA | NA | 0.0003339120 | 0.0003339120 |
| gnom AD | 0.0001145740 | NA | NA | 0.0001145740 |
| gnom AD | NA | NA | 0.0000667111 | 0.0000667111 |
| 1KGP | NA | 0.4612000000 | NA | NA |
| AGVP | 0.3031250000 | NA | NA | NA |
| gnom AD | 0.2370170000 | NA | 0.4472210000 | 0.8069310000 |
| 1KGP | NA | 0.0040000000 | NA | NA |
| gnom AD | 0.0008022000 | NA | 0.0051965400 | 0.0051965400 |
| gnom AD | NA | NA | NA | 0.0006165230 |
| gnom AD | 0.0002294100 | NA | NA | 0.0002294100 |
| 1KGP | 0.0703000000 | NA | NA | NA |
| AGVP | 0.0437500000 | NA | NA | NA |
| gnom AD | 0.0629017000 | NA | 0.0001333870 | 0.0629017000 |
| 1KGP | NA | NA | NA | NA |
| 1KGP | 0.0159000000 | NA | NA | NA |
| gnom AD | 0.0199496000 | NA | NA | 0.0199496000 |
| gnom AD | 0.0001147320 | NA | NA | 0.0001147320 |
| gnom AD | NA | NA | 0.0000905797 | 0.0000905797 |
| gnom AD | NA | NA | 0.0000684838 | 0.0000684838 |
| gnom AD | NA | NA | 0.0000680643 | 0.0000680643 |
| gnom AD | 0.0001221600 | NA | NA | 0.0001221600 |
| 1KGP | 0.0008000000 | NA | NA | NA |
| gnom AD | 0.0002289380 | NA | NA | 0.0002289380 |
| gnom AD | 0.0001144950 | NA | NA | 0.0001144950 |

Source—source for variation.
identfier—identifier of the gRNA molecule targeting domain
Chr—chromosome on which the gRNA and variation are located
Pos—position of the variant, hg19
Id—if the variant is a known SNP, the rs identifier
Ref—reference allele sequence
Alt—alternate allele sequence
Quality—quality score associated with the variant, higher is better
AF—allele frequency of the variant across all samples/populations
AF_AFR—allele frequency in African populations
AF_EUR—allele frequency in European populations
AF_NFE—allele frequency in non-Finish European populations
AF_POPMAX—maximum allele frequency across the available populations

That variant sequences were identified within the target sequences of specific gene editing reagents such as those assessed here supports the methods described herein, for example, methods of treating cells or patients with gene editing systems (e.g., as described herein), said methods comprising a step of assaying the target cell/patient of the presence of a fully complementary target sequence, and on the basis of identifying a fully complementary target sequence at the intended location, treating the cell/patient, e.g., as described herein.

To the extent there are any discrepancies between any sequence listing and any sequence recited in the specification, the sequence recited in the specification should be considered the correct sequence. Unless otherwise indicated, all genomic locations are according to hg38.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A method of selectively treating a patient with a gene editing system, comprising:
e) selectively introducing said gene editing system into a cell, e.g., population of cells, of the patient on the basis of the cell, e.g., population of cells, comprising a target sequence, at a target locus, that is fully complementary to a targeting domain of said gene editing system; and/or
f) selectively introducing said gene editing system to a cell, e.g., population of cells, of the patient on the basis of the cell, e.g., population of cells, not comprising a target sequence, at a locus other than the target locus, that is fully complementary to a targeting domain of said gene editing system.

2. A method of selectively treating a patient with a gene editing system, comprising:
a) selecting the patient for treatment on the basis of one or more cells of the patient comprising a target sequence, at a target locus, that is fully complementary to a targeting domain of said gene editing system; and
b) thereafter, administering a therapeutically effective amount of said gene editing system to the patient or to a population of cells of said patient,
thereby inducing a modification at or near the target sequence at the target locus in a cell or the patient or a cell of the population of cells.

3. A method of selectively treating a patient with a gene editing system comprising:
a) assaying one or more cells from a biological sample from the patient for the presence of a target sequence, at a target locus, that is fully complementary to a targeting domain of said gene editing system; and
b) thereafter, selectively administering a therapeutically effective amount of the gene editing system to the patient or to a cell of the patient:
i) on the basis of one or more cells of the biological sample of the patient comprising a target sequence, at a target locus, that is fully complementary to a targeting domain of said gene editing system; and/or
ii) on the basis of one or more cells of the biological sample from the patient not comprising a target sequence, at a locus other than the target locus, that is fully complementary to a targeting domain of said gene editing system,
thereby inducing a modification at or near the target sequence at the target locus in a cell or the patient or a cell of the population of cells.

4. A method of selectively treating a patient with a gene editing system, comprising:
a) assaying one or more cells of a biological sample from the patient for at least one target sequence, at a target locus, that is fully complementary to the targeting domain of said gene editing system;
b) thereafter, selecting the patient for treatment with the gene editing system on the basis of one or more cells of the biological sample from the patient having the target sequence, at the target locus, that is fully complementary to the targeting domain of said gene editing system; and
c) thereafter, administering a therapeutically effective amount of the gene editing system of cells to the patient.

5. The method according to any one of claims 3-4, wherein the biological sample is selected from the group consisting of synovial fluid, blood, bone marrow, serum, feces, plasma, urine, tear, saliva, cerebrospinal fluid, an apheresis sample, a leukopheresis sample, a leukocyte sample and a tissue sample.

6. The method of claim 5, wherein the biological sample is blood, an apheresis sample, a leukopheresis sample, a leukocyte sample, or bone marrow.

7. The method according to any one of claims 3-6, wherein the step of assaying comprises a technique selected from the group consisting of Next generation sequencing (NGS), pyrosequencing, Sanger sequencing, Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southern Blot, immunoassays, immunohistochemistry, ELISA, flow cytometry, Western blot, HPLC, and mass spectrometry.

8. The method according to any one of claims 1-7, wherein the one or more cells comprise, e.g., consist of, hematopoietic stem and progenitor cells (HSPCs) or HSCs.

9. The method according to any one of claims 1-8, wherein the patient has a hemoglobinopathy.

10. The method according to claim 9, wherein the hemoglobinopathy is sickle cell disease, sickle cell anemia, beta-thalassemia, thalassemia major, thalassemia intermedia.

11. The method according to any of claims 9-10, wherein the target locus is the human globin locus.

12. The method of claim 11, wherein the target locus is the HBG1 promoter (Chr11:5,249,833-5,250,237 according to hg38) and/or HBG2 promoter (Chr11:5,254,738-5,255,164 according to hg38).

13. The method of claim 11, wherein the target locus is an HPFH region.

14. The method according to any of claims 9-10, wherein the target locus is an AAVS1 locus.

15. The method according to any of claims 9-10, wherein the target locus is a BCL11a gene.

16. The method according to any of claims 9-10, wherein the target locus is a BCL11a enhancer region.

17. The method according to claim 16, wherein the target locus is:
a) the +55 region of the BCL11a enhancer (Chr2:60497676-60498941 according to hg38);
b) the +58 region of the BCL11a enhancer (Chr2:60494251-60495546 according to hg38); or
c) the +62 region of the BCL11a enhancer (Chr2:60490409-60491734 according to hg38).

18. The method of any of claims 1-17, wherein the gene editing system comprises:
a) a zinc finger nuclease (ZFN) system;
b) a TALEN system;
c) a meganuclease system; or
d) a CRISPR system.

19. The method of claim 18, wherein the gene editing system comprises a CRISPR system comprising a gRNA molecule comprising a targeting domain complementary to any one of SEQ ID NO: 1 to 161,197 of PCT Publication WO2017/077394.

20. The method of claim 18, wherein the gene editing system comprises a CRISPR system comprising a gRNA molecule comprising a targeting domain complementary to any one of SEQ ID NO: 1 to 135 of PCT Publication WO2016/182917.

21. The method of claim 18, wherein the gene editing system comprises a ZFN system comprising a targeting domain complementary to any one of SEQ ID NO: 63-80 and 232-251 of PCT Publication WO2015/073683.

22. The method of claim 18, wherein the gene editing system comprises a TALEN system comprising a targeting domain complementary to any one of SEQ ID NO: 7-11, 16-62, and 143-184 of PCT Publication WO2015/073683.

23. The method according to any one of claims 1-7, wherein the one or more cells comprise, e.g., consist of, T cells.

24. The method according to any one of claims 1-7 and 23, wherein the patient has a cancer or autoimmune disease.

25. The method according to any one of claims 1-7 and 23, wherein the patient has a cancer.

26. The method according to any one of claims 23-25, wherein the target locus is selected from the group consisting of: TRAC, TRBC1, TRBC2, CD3E, CD3G, CD3D, B2M, CIITA, CD247, HLA-A, HLA-B, HLA-C, DCK, CD52, FKBP1A, NLRC5, RFXANK, RFX5, RFXAP, NR3C1, CD274, HAVCR2, LAG3, PDCD1, PD-L2, CTLA4, CEACAM (e.g., CEACAM-1, CEACAM-3 and/or CEACAM-5), VISTA, BTLA, TIGIT, LAIR1, CD160, 2B4, CD80, CD86, B7-H3 (CD113), B7-H4 (VTCN1), HVEM (TNFRSF14 or CD107), KIR, A2aR, MHC class I, MHC class II, GAL9, adenosine, and TGF beta, PTPN11, and combinations thereof.

27. The method of any one of claims 23-26, wherein the gene editing system comprises:
e) a zinc finger nuclease (ZFN) system;
f) a TALEN system;
g) a meganuclease system; or
h) a CRISPR system.

28. The method of claim 27, wherein the gene editing system comprises a CRISPR system comprising a gRNA molecule comprising a targeting domain described in PCT Publication WO/2017/093969, for example, described in any of Tables 1-6 and 6b-g of WO2017/093969.

29. A gene editing system for use in treating a patient having a disease, characterized in that a therapeutically effective amount of the gene editing system is to be administered to the patient (or cells of the patient) on the basis of a cell of said patient comprising a target sequence, at a target locus, that is fully complementary to a targeting domain of said gene editing system.

30. A gene editing system for use in treating a patient having a disease, characterized in that:
a) the patient is to be selected for treatment with the gene editing system on the basis of a cell of said patient comprising a target sequence, at a target locus, that is fully complementary to a targeting domain of said gene editing system; and
b) thereafter, a therapeutically effective amount of the gene editing system is to be administered to the patient.

31. A gene editing system for use in treating a patient having a disease, characterized in that:
a) a cell of a biological sample from the patient is to be assayed for at least one a target sequence, at a target locus, that is fully complementary to a targeting domain of said gene editing system; and
b) a therapeutically effective amount of the gene editing system is to be selectively administered to the patient on the basis of the cell of the biological sample from the patient having the at least one a target sequence, at a target locus, that is fully complementary to a targeting domain of said gene editing system.

32. A gene editing system for use in treating a patient having a disease, characterized in that:
e) a cell of a biological sample from the patient is to be assayed for at least one a target sequence, at a target locus, that is fully complementary to a targeting domain of said gene editing system;
f) the patient is selected for treatment with the gene editing system on the basis of the cell of the biological sample from the patient having the at least one a target sequence, at a target locus, that is fully complementary to a targeting domain of said gene editing system; and
c) a therapeutically effective amount of the gene editing system is to be selectively administered to the patient.

33. A method of predicting the likelihood that a patient having an disease will respond to treatment with a gene editing system, comprising assaying a cell of a biological sample from the patient for the presence or absence of at least one target sequence, at a target locus, that is fully complementary to a targeting domain of said gene editing system, wherein:
a) the presence of the at least one target sequence, at a target locus, that is fully complementary to a targeting domain of said gene editing system is indicative of an increased likelihood that the patient will respond to treatment with the gene editing system; and
b) the absence of the at least one target sequence, at a target locus, that is fully complementary to a targeting domain of said gene editing system is indicative of a decreased likelihood that the patient will respond to treatment with the gene editing system.

34. The method according to claim 33, further comprising the step of obtaining the biological sample from the patient, wherein the step of obtaining is performed prior to the step of assaying.

35. The method according to any one of claims 33-34, wherein the biological sample is selected from the group consisting of synovial fluid, blood, bone marrow, serum, feces, plasma, urine, tear, saliva, cerebrospinal fluid, an apheresis sample, a leukopheresis sample, a leukocyte sample and a tissue sample.

36. The method of claim 35, wherein the biological sample is blood, an apheresis sample, a leukopheresis sample, a leukocyte sample, or bone marrow.

37. The method according to any one of claims 33-36, wherein the step of assaying comprises a technique selected from the group consisting of Next generation sequencing (NGS), pyrosequencing, Sanger sequencing, Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southern Blot, immunoassays, immunohistochemistry, ELISA, flow cytometry, Western blot, HPLC, and mass spectrometry.

38. The method according to any one of claims 33-37, wherein the one or more cells comprise, e.g., consist of, hematopoietic stem and progenitor cells (HSPCs) or HSCs.

39. The method according to any one of claims 33-37, wherein the one or more cells comprise, e.g., consist of, T cells.

40. The method or gene editing system for use of any of claims 29-39, wherein the gene editing system comprises:
a) a zinc finger nuclease (ZFN) system;
b) a TALEN system;
c) a meganuclease system; or
d) a CRISPR system.

41. The method according to claim 11, wherein the CRISPR system comprises a gRNA comprising a targeting domain sequence selected from the targeting domain sequences of Tables 1-3.
